(19) United States
(12) Patent Application Publication
(10) Pub. No.: US 2024/0390295 A1
(43) Pub. Date: Nov. 28, 2024

(54) METHODS FOR TREATING DEPRESSION

(71) Applicant: JANSSEN PHARMACEUTICA NV, Beerse (BE)

(72) Inventors: David James WILLIAMSON, Titusville, NJ (US); Ella DALY, Titusville, NJ (US)

(21) Appl. No.: 17/999,581

(22) PCT Filed: May 7, 2021
(86) PCT No.: PCT/US2021/031360

§ 371 Date: Nov. 22, 2022

Related U.S. Application Data
(60) Provisional application No. 63/031,346, filed on May 28, 2020.

(51) Int. Cl. A61K 31/135; A61K 9/00; A61P 25/24

(52) U.S. Cl. CPC A61K 31/135; A61K 9/0043; A61P 25/24

(57) ABSTRACT

The disclosure is directed to methods of treating depression in a human patient in need thereof, whereby certain patients may be qualified for a reduced monitoring period after a treatment session. The methods are based on an analysis of adverse events and provide treatment options for the patient and healthcare provider.

### CROSS-REFERENCE TO RELATED APPLICATIONS

This application claims the benefit of the priority to U.S. Provisional Patent Application No. 63/031,346, filed May 28, 2020, the disclosure of which is incorporated herein by reference.

### TECHNICAL FIELD

This invention relates to methods of treating depression.

### BACKGROUND

The skillful use of medications in psychiatry requires a balance between effectively treating the underlying condition(s) and minimizing adverse events (AEs). Balancing of the risk-benefit equation is typically undertaken in collaboration with the patient, with the clinician contributing knowledge of the typical profile of the medication, and the patient contributing their individual experience of a medication's positive versus unwanted effects.

Every patient is not equally susceptible to each possible AE that can occur with a treatment; however, the average incidence rates listed in a medication's product labeling are often the only guidance available to the clinician. Furthermore, these rates do not dynamically adapt to how well a patient tolerates a medication over repeated administration: such adaptability is an essential aspect of individualizing the medication to each patient. Thus, a useful addition to the summary-level information provided in product labeling would be data informing the clinician how a medication's AE profile changes over time and the extent to which patient tolerability early in the course of treatment provides insight into how AEs may manifest as treatment progresses.

Esketamine (ESK) is a noncompetitive N-methyl-D-aspartate (NMDA) receptor antagonist approved, in conjunction with a Food and Drug Administration approved oral antidepressant (OAD), for the treatment of adults with treatment-resistant major depressive disorder (TRD). There is a need in the art for adjusting treatment protocols for patients who respond favorably to esketamine during treatment.

### SUMMARY

In some embodiments, the disclosure is directed to methods of treating depression in a human patient in need thereof, wherein the method has an induction phase and treatment sessions, and wherein the induction phase has a duration of 4 weeks. Prior to any treatment session, the patient has a systolic blood pressure of less than 140 mmHg and a diastolic blood pressure of less than 110 mmHg. The methods comprise intranasally administering about 56 mg or about 84 mg of esketamine per induction phase treatment session to said patient, wherein the induction phase treatment session occurs at a frequency of twice weekly during the induction phase. After at least the first two treatment sessions, the patient is monitored for a period of at least 90 minutes for adverse events. If the patient does not experience a severe adverse event and does not experience any clinically meaningful blood pressure increases, clinically meaningful heart rate increases, moderate or greater levels of dissociation, moderate or greater levels of disorientation, moderate or greater levels of sedation, moderate or greater levels of dizziness, moderate or greater levels of nausea without vomiting, and/or moderate of greater levels of vertigo, following any two consecutive treatment sessions, the patient becomes a qualified patient for a post-treatment session monitoring period of less than 90 minutes in a next treatment session. In some aspects, the depression is major depressive disorder, major depressive disorder with suicidal ideation, or treatment resistant depression. For example, the method is suitable to treat severe major depressive disorder when urgent symptom control is necessary.

The methods are applicable to patients who do not have current uncontrolled hypertension, patients not taking medication or substances that promote sedation or blood pressure increases, and/or patients less than 65 years of age.

In other embodiments, the methods further comprise a subsequent maintenance phase comprising intranasally administering about 56 mg or about 84 mg of esketamine per maintenance phase treatment session to said patient, wherein the maintenance phase treatment session occurs at a frequency of once weekly for the first four weeks of the maintenance phase and is adjusted to once weekly or once every other week thereafter.

### BRIEF DESCRIPTION OF THE DRAWINGS

FIG. 1 is a schematic of the patients included in Example 2.

FIG. 2A is a line graph showing percentage of esketamine-treated participants with adverse events based on frequency of Week 1 and Week 4 occurrence, for clinician-reported dissociation. FIG. 2B is a line graph showing Percentage of esketamine-treated participants with adverse events based on frequency of Week 1 and Week 4 occurrence, for clinician-reported sedation. FIG. 2C is a line graph showing Percentage of esketamine-treated participants with adverse events based on frequency of Week 1 and Week 4 occurrence, for clinician-reported increased blood pressure.

FIG. 3A are line graphs showing the percentage of esketamine-treated participants with adverse events based on frequency of Week 1 and Week 4 occurrence for CADSS-based dissociation. FIG. 3B are line graphs showing the percentage of esketamine-treated participants with adverse events based on frequency of Week 1 and Week 4 occurrence for MOASS-based sedation. FIG. 3C are line graphs showing the percentage of esketamine-treated participants with adverse events based on frequency of Week 1 and Week 4 occurrence for measure-based increased blood pressure. FIG. 3D are line graphs showing the percentage of esketamine-treated participants with adverse events based on frequency of Week 1 and Week 4 occurrence for clinician-reported dizziness. FIG. 3E are line graphs showing the percentage of esketamine-treated participants with adverse events based on frequency of Week 1 and Week 4 occurrence for clinician-reported nausea. FIG. 3F are line graphs showing the percentage of esketamine-treated participants with adverse events based on frequency of Week 1 and Week 4 occurrence for clinician-reported vertigo.

FIG. 4A is a line graph showing the percentage of esketamine-treated patients with adverse events based on esketamine nasal spray dose, for clinician-reported dissociation. FIG. 4B is a line graph showing the percentage of esketamine-treated patients with adverse events based on esketamine nasal spray dose, for clinician-reported sedation. FIG. 4C is a line graph showing the percentage of esketamine-treated patients with adverse events based on esketamine nasal spray dose, for clinician-reported increased blood pressure. FIG. 4D is a line graph showing the percentage of esketamine-treated patients with adverse events based on esketamine nasal spray dose, for clinician-reported dizziness. FIG. 4E is a line graph showing the percentage of esketamine-treated patients with adverse events based on esketamine nasal spray dose, for clinician-reported nausea. FIG. 4F is a line graph showing the percentage of esketamine-treated patients with adverse events based on esketamine nasal spray dose, for clinician-reported vertigo. FIG. 4G is a line graph showing the percentage of esketamine-treated patients with adverse events based on esketamine nasal spray dose, for CADSS-based dissociation. FIG. 4H is a line graph showing the percentage of esketamine-treated patients with adverse events based on esketamine nasal spray dose for MOASS-based sedation. FIG. 4I is a line graph showing the percentage of esketamine-treated patients with adverse events based on esketamine nasal spray dose for measure-based increased blood pressure

### DETAILED DESCRIPTION OF ILLUSTRATIVE EMBODIMENTS

In one aspect of the present invention, proper precautions are disclosed to deal with patients who are at risk for rare side effects, and a shorter period of post-treatment session (post-dose) monitoring is disclosed for qualified patients. By monitoring and releasing patients in a shorter period, time and resources are saved. A part of developing esketamine dosing instructions is understanding the onset of side effects that may exist for certain patient populations that may require extra precaution to be taken for that class of patient. Thus, the methods discussed herein facilitate conversations between the clinician and patient about the likelihood of recurrence of specific AEs during future treatment sessions with ESK, thereby facilitating treatment compliance and determining the treatment's likely acceptability to a patient. The methods also are useful in framing the clinician's thinking about which strategies may be most relevant to a given patient's management in future dosing sessions. By doing so, the patient's treatment is personalized.

It has been discovered that the more frequent an AE occurred to a patient during the first two treatment session (e.g. first week of treatment), the higher the percentage of the that patient suffering a recurrence. A similar pattern of AEs was found at Week 4, with the pattern at Week 4 being more predictive of subsequent AEs. For example, the rate of clinician-reported dissociation during Weeks 2-4 for overall patient population was 16.1% compared with 86.5% (64/74 patients) for those reporting dissociation twice in Week 1. 48.2% (41/85 patients) for those who reported dissociation once in Week 1 and 5.6% (44/790) for those who did not report dissociation once in Week 1. For all clinician-reported AEs examined, at least 78% of patients were in the category associated with fewest recurrence, i.e., AE not reported during Week 1. After longer periods drug administration (e.g. 4 weeks, 8 weeks or 12 weeks) the severity of all serious AE also declines. Thus, based on the data that has been amassed on AEs it is possible to stratify patients related to the different frequencies of AE recurrence. The practical implication of these finding is that patient stratification coupled with knowledge of AE onset data may be used to stratify patients into different groups and those qualified patients with lower AE frequencies should require shorter monitoring periods for AE before being assessed by physician for release.

The methods described herein are directed to treating depression (i.e. depressive disorders such as major depressive disorder (MDD), treatment resistant depression (TRD), major depressive disorder with suicidal ideation (MDSI)), or severe major depressive disorder in a human patient in need thereof. The methods desirably permit certain patients, i.e., qualified patients, to require shorter post-treatment session (post-dose) monitoring periods and the ability to leave the treating site earlier than unqualified patients or the period that may have been established as a conventional monitoring period (or in prior product labeling). Typically, such patients are considered clinically stable based on clinical judgment.

As used herein, unless otherwise noted, the terms “subject” and “patient” refer to a human, who has been the object of treatment, observation or experiment. Preferably, the patient has experienced and/or exhibited at least one symptom of the disease or disorder to be treated and/or prevented. In some embodiments, the patient is an adult. As used herein, the term “adult” as used herein refers to a human that is about less than 65 years of age. In other embodiments, the term “adult” refers to a human patient that is 18 years of age to about 64 years of age. As used herein, the term “elderly” is over 65 years of age.

As used herein, the term “depression” (also referred to as depressive disorder) includes major depressive disorder, persistent depressive disorder, seasonal affective disorder, postpartum depression, premenstrual dysphoric disorder, situational depression, anhedonia, melancholy, mid-life depression, late-life depression, depression due to identifiable stressors, treatment resistant depression, or combinations thereof. In certain embodiments, the depression is major depressive disorder. In other embodiments, the major depressive disorder is with melancholic features or anxious distress. In further embodiments, the depression is treatment-resistant depression. In other embodiments, the depression is major depressive disorder with suicidal ideation.

As known in the art, a patient is considered to have major depressive disorder if exhibiting five or more symptoms during the same two week period that are a change from previous functioning: depressed mood and/or loss of interest/pleasure must be present: excluding symptoms clearly attributable to another medical condition.
1. Depressed mood: Most of the day, nearly every day; may be subjective (e.g. feels sad, empty, hopeless) or observed by others (e.g. appears tearful); in children and adolescents, can be irritable mood
2. Loss of interest/pleasure: Markedly diminished interest/pleasure in all (or almost all) activities most of the day, nearly every day; may be subjective or observed by others
3. Weight loss or gain: Significant weight loss (without dieting) or gain (change of >5% body weight in a month), or decrease or increase in appetite nearly every day; in children, may be failure to gain weight as expected
4. Insomnia or hypersomnia: Nearly every day
5. Psychomotor agitation or retardation: Nearly every day and observable by others (not merely subjectively restless or slow)
6. Fatigue: Or loss of energy, nearly every day
7. Feeling worthless or excessive/inappropriate guilt: Nearly every day; guilt may be delusional: not merely self reproach or guilt about being sick
8. Decreased concentration: Nearly every day; may be indecisiveness; may be subjective or observed by others
9. Thoughts of death/suicide” Recurrent thoughts of death (not just fear of dying), recurrent suicidal ideation without specific plan, or suicide attempt, or a specific plan for suicide
   To be diagnosed with MDD, the following criteria also must be met:
1. Symptoms cause clinically significant distress or impairment in social, occupational, or other important areas of functioning
2. Episode not attributable to physiological effects of a substance or another medical condition
3. Episode not better explained by schizoaffective disorder, schizophrenia, schizophreniform disorder, delusional disorder, or other specified and unspecified schizophrenia spectrum and other psychotic disorders
4. No history of manic or hypomanic episode

Major depressive disorder may be categorized as mild, moderate, or severe. In some embodiments, the MDD is mild. In other embodiments, the MDD is moderate. In further embodiments, the MDD is severe. As used herein, “mild MDD” applies to a patient having few, if any symptoms in excess of those required to make the diagnosis, the intensity of the symptoms is distressing but manageable, and the symptoms result in minor impairment in social or occupational functioning. The mild MDD may be a single episode (DSM ICD-10 F32.0) or a recurrent episode (DSM ICD-10 F33.0). “Moderate MDD” applies to a patient having a number of symptoms, intensity of symptoms, and/or functional impairment are between those specified for “mild” and “severe.” The moderate MDD may be a single episode (DSM ICD-10 F32.1) or a recurrent episode (DSM ICD-10 F33.1). “Severe MDD” applies to a patient where the number of symptoms is substantially in excess of that required to make the diagnosis, the intensity of symptoms is seriously distressing and unmanageable, and the symptoms markedly interfere with social and occupational functioning, and urgent symptom control is necessary. In some embodiments, the severe MDD may be a single episode (DSM ICD-10 F32.2) or a recurrent episode (DSM ICD-10 F33.2).

As used herein, the term “episode of major depressive disorder” means a continuous period (e.g., about 2 weeks or more) in which a patient has symptoms of a major depressive disorder sufficient to meet criteria for major depression as specified in the Diagnostic and Statistical Manual of Mental Disorders, 5th Edition: DSM 5.

As used herein, the term “treatment-refractory or treatment-resistant depression” and the abbreviation “TRD” shall be defined as major depressive disorder in a patient that does not respond adequately to at least two different antidepressants, preferably between two and five antidepressants, in the current depressive episode. Such patients may be in need of immediate treatment for severe depression or severe episode of depression. In other embodiments, TRD is defined as major depressive disorder in a patient that has not responded to at least two oral antidepressants of adequate dose and duration in the current depressive episode.

As used herein, “suicide” is the “act of taking one's own life”. See, http://en.wikipedia.org/wiki/Suicide-cite_note-7. Suicide includes attempted suicide or non-fatal suicidal behavior, which is self-injury with the desire to end one's life that does not result in death. Suicide attempt is a self-initiated sequence of behaviors by an individual who, at the time of initiation, expected that the set of actions would lead to his or her own death.

As used herein, “suicidal ideation” refers to thoughts about or an unusual preoccupation with suicide, or thoughts of ending one's life or not wanting to live anymore but not necessarily taking any active efforts to do so. The range of suicidal ideation varies greatly from fleeting to chronic and progresses to detailed planning, role playing, and unsuccessful attempts, which may be deliberately constructed to fail or be discovered, or may be fully intended to result in death. In some embodiments, a patient is classified as being “suicidal” when the patient has a mean baseline MADRS total score of about 38 or greater. In other embodiments, a patient is classified as being suicidal when the patient has a mean baseline BBSS score of 22 or greater. In further embodiments, a patient is classified as being suicidal when the patient has a score of 6 or greater in the SIBAT clinical global judgement of suicide risk. In yet other embodiments, the patient has one or more combinations of these scores. “Suicidal ideation with intent” may be confirmed through questioning of the patient in view of the scales/tools disclosed herein, and includes thinking (even momentarily) about harming or of hurting or of injuring oneself, with at least some intent or awareness that you might die as a result: or thinking about suicide (i.e., about killing oneself, and intending to act on thoughts of killing oneself).

Prior to any treatment session described herein, the patient, for safety reasons, should have a systolic blood pressure of less than about 140 mmHg and a diastolic blood pressure of less than about 110 mmHg, preferably less than about 100 mmHg and more preferably less than or equal to about 90 mmHg. In some embodiments, the patient has a systolic blood pressure of less than about 140 mmHg, about 138 mmHg, about 135 mmHg, 130 mmHg. 125 mmHg, 120 mmHg, 115 mmHg, 100 mmHg, 110 mmHg, 90 mmHg, or less providing that the systolic blood pressure is considered normal per clinical judgment. In further embodiments, the systolic blood pressure is about 90 to about 135, about 90 to about 130, about 90 to about 120, about 90 to about 110, about 90 to about 100, about 100 to about 135, about 100 to about 130, about 100 to about 120, about 100 to about 110, about 110 to about 135, about 110 to about 130, about 110 to about 120, about 120 to about 135, or about 130 to about 135 mmHg. In other aspects, the patient's diastolic blood pressure is less than about 110 mmHg. In yet other embodiments, the patient's diastolic blood pressure is less than about 110, 100, 105, 100, 90, 80, 70, 60, or 50, providing that the diastolic blood pressure is considered normal. In still further embodiments, the patient's diastolic blood pressure is about 50 to about 110, about 50 to about 100, about 50 to about 90, about 50 to about 80, about 50 to about 70, about 50 to about 60, about 60 to about 110, about 60 to about 100, about 60 to about 90, about 60 to about 80, about 60 to about 70, about 70 to about 110, about 70 to about 100, about 70 to about 90, about 70 to about 80, about 80 to about 110, about 80) to about 100, about 80 to about 90, about 90 to about 110, or about 90 to about 100 mmHg. If the patient does not have a systolic blood pressure of less than 140 mmHg and a diastolic blood pressure of less than 110 mmHg, the treatment session should be rescheduled.

Desirably, the patient does not have current uncontrolled hypertension prior to or after administration esketamine. The term “hypertension” or “high blood pressure” as used herein are interchangeable and refers to a patient having stage two hypertension, i.e., a systolic pressure of about 140 mmHg or higher. Typically, hypertension that is “uncontrolled” is understood to mean that the systolic pressure cannot be lowered to less than about 140 mmHg using medication, engaging in exercise, following special diets, limiting alcohol, lowering body weight, eliminating tobacco use, or other factors know to contribute hypertension. In some embodiments, the hypertension is in the context of cardiovascular co-morbidity, endocrine co-morbidity, or combinations thereof.

Prior to qualifying for reduced post-treatment session monitoring periods, the patient, or a caregiver in some embodiments, provides consent to the methods described herein, i.e., informed consent. By doing so, the patient is aware of the implications of the treatment methods including possible adverse events, if any. In some embodiments, informed consent is documented, i.e., includes the execution of forms agreeing to one or more guidelines regarding the disclosed methods. In other embodiments, informed consent includes verbal agreement of one or more guidelines regarding the methods described herein. In general, the patient is made aware of the potential risks related to monitoring for less than 2 hours. In particular, the patient is made aware of the potential risks and agree to proceed with administration of esketamine and monitoring for less than 2 hours, if qualified. In some aspects, the patient agrees to comply with the instructions provided by the clinician if the patient does not feel well or has any other relevant health issues following the post-treatment session monitoring period. In other aspects, the caregiver or responsible adult agrees to remain with the patient after the treatment session has ended, after the post-treatment session monitoring period has ended, and until the agreed upon follow-up assessment, i.e., telephone, with the clinician has occurred, preferably at least 2 hours after the end of the treatment session, later the same day. In further aspects, the clinician provides instructions to the patient and caregiver or responsible adult based on the healthcare setting's processes and procedures, in the event the patient requires medical intervention after the patient and adult caregiver/responsible adult leave the clinic. In vet other aspects, if the patient requires medical intervention, the patient agrees to comply with the instructions provided by clinician.

The attending physician, however, may use clinical judgment in determining whether to prescribe certain concomitant medication because of their possible effects on patients taking esketamine. For example, certain concomitant/contraindicated medications, opioids, and alcohol may result in adverse effects such as sedation that may impact a patient's ability to become or remain a qualified patient. Thus, restricting, minimizing or eliminating administration of such concomitant/contraindicated medications, opioids, and alcohol helps to qualify a patient for the treatment methods described herein and retain the patient as qualified. In some embodiments, the concomitant medication is capable of promoting dizziness, sedation, i.e., sedatives, or an increase in blood pressure. In other embodiments, the concomitant medications include, without limitation, CNS depressants (e.g., benzodiazepines, opioids, alcohol), psychostimulants (e.g., amphetamines, methylphenidate, modafanil, armodafinil), and monoamine oxidase inhibitors (MAOI) inhibitors. Examples of benzodiazepines include diazepam (Zetran), estazolam (Prosom (Pro)), quazepam (Doral (Pro)), alprazolam (Niravam (Pro)), diazepam (Diazepam Intensol), alprazolam (Alprazolam Intensol (Pro)), clorazepate (Tranxene), alprazolam (Xanax XR (Pro)), clonazepam (Klonopin Wafer), chlordiazepoxide (Librium (Pro)), oxazepam (Serax), alprazolam (Xanax (Pro)), lorazepam (Lorazepam Intensol (Pro)), flurazepam (Dalmane), clonazepam (Klonopin (Pro)), diazepam (Valium (Pro)), triazolam (Halcion (Pro)), lorazepam (Ativan (Pro)), clorazepate (Tranxene T-Tab (Pro)), temazepam (Restoril (Pro)), clorazepate (Tranxene SD), midazolam (Versed), midazolam (Nayzilam (Pro)), and midazolam (Seizalam).

The terms “adverse event”, “side effects”, and “AE” are used interchangeably herein and refer to an untoward medical occurrence. An adverse event is any unfavorable and unintended sign, symptom, or disease temporally associated with the methods herein. In some embodiments, the adverse event is not related with the methods described herein. In other embodiments, the adverse event is related with the methods described herein. The adverse event includes any occurrence that is new in onset or aggravated in severity or frequency from the baseline condition, or abnormal results of diagnostic procedures, including laboratory test abnormalities. In some embodiments, the adverse event is a cardiac disorder such as tachycardia. In other embodiments, the adverse event is an ear and labyrinth disorder such as vertigo. In further embodiments, the adverse event is a gastrointestinal disorder such as constipation, diarrhea, dry mouth, nausea, or vomiting, or a combination thereof. In still other embodiments, the adverse event is a general disorder/administration site conditions such as feeling abnormal, feeling drunk, or a combination thereof. In yet further embodiments, the adverse event is a blood pressure increase. In still further embodiments, the adverse event is a clinically meaningful heart rate increase. In other embodiments, the adverse event is a nervous system disorder such as dizziness, dysarthria, dysgeusia, headache, hypoesthesia, lethargy, mental impairment, sedation, or tremor, or combinations thereof. In further embodiments, the adverse event is a psychiatric disorder that is anxiety, dissociation, euphoric mood, insomnia, or a combination thereof. In still other embodiments, the adverse event is a renal and urinary disorder such as pollakiuria. In yet further embodiments, the adverse event is a respiratory, thoracic and mediastinal disorder such as nasal discomfort, oropharyngeal pain, throat irritation, or combinations thereof. In other embodiments, the adverse event is a skin and subcutaneous tissue disorders such as hyperhidrosis. In further embodiments, the adverse event is disorientation.

Anxiety includes agitation, anticipatory anxiety, anxiety, fear, feeling jittery, irritability, nervousness, panic attack, tension, or combinations thereof. In some embodiment, the adverse event is agitation. In other embodiments, the adverse event is anticipatory anxiety. In further embodiments, the adverse event is normal anxiety. In still other embodiments, the adverse event is feeling jittery. In yet further embodiments, the adverse event is fear. In other embodiments, the anxiety is irritability. In further embodiments, the adverse event is nervousness. In yet other embodiments, the adverse event is panic attack. In still further embodiments, the anxiety is tension.

“Blood pressure increase” or variations thereof includes an increase in diastolic blood pressure diastolic, increase in overall blood pressure, increase in systolic blood pressure, hypertension, or combinations thereof. In some embodiments, the adverse event is an increase in diastolic blood pressure. In further embodiments, the adverse event is an increase in overall blood pressure. In other embodiments, the adverse event is an increase in systolic blood pressure. In still further embodiments, the adverse event is hypertension.

Disorientation includes a transient state of confusion, especially as to time, place or identity as a result of medication. One of skill in the art would be able to identify disorientation in a patient.

Dissociation, i.e., feeling disconnected from space and/or time, includes delusional perception, depersonalization/derealization disorder, derealization, diplopia, dysesthesia, feeling cold, feeling hot, feeling of body temperature change, hallucination, hallucination/auditory, hallucination/visual, hyperacusis, illusion, ocular discomfort, oral dysesthesia, paresthesia, paresthesia oral, pharyngeal paresthesia, photophobia, time perception altered, tinnitus, vision blurred, visual impairment, or combinations thereof. In some embodiments, the adverse event is delusional perception. In other embodiments, the adverse event is depersonalization/derealization disorder. In further embodiments, the adverse event is derealization. In yet other embodiments, the adverse event is diplopia. In still further embodiments, the adverse event is dissociation. In still further embodiments, the adverse event is dysesthesia. In other embodiments, the adverse event is feeling cold. In further embodiments, the adverse event is feeling hot. In yet other embodiments, the adverse event is feeling of body temperature change. In still further embodiments, the adverse event is hallucination. In other embodiments, the adverse event is auditory hallucination. In further embodiments, the adverse event is visual hallucination. In still other embodiments, the adverse event is hyperacusis. In yet further embodiments, the adverse event is illusion. In other embodiments, the adverse event is ocular discomfort. In further embodiments, the adverse event is oral dysesthesia. In still other embodiments, the adverse event is paresthesia. In yet further embodiments, the adverse event is oral paresthesia. In yet further embodiments, the adverse event is pharyngeal paresthesia. In other embodiments, the adverse event is photophobia. In further embodiments, the adverse event is an alteration of time perception. In yet other embodiments, the adverse event is tinnitus. In still further embodiments, the adverse event is vision blurred. In still further embodiments, the adverse event is visual impairment.

Dizziness includes dizziness, exertional dizziness, postural dizziness, procedural dizziness, or a combination thereof. In some embodiments, the adverse event is dizziness. In other embodiments, the adverse event is exertional dizziness. In further embodiments, the adverse event is postural dizziness. In still other embodiments, the adverse event is procedural dizziness.

Dysarthria includes dysarthria, slow speech, speech disorder, or a combination thereof. In some embodiments, the adverse event is dysarthria. In further embodiments, the adverse event is slow speech. In other embodiments, the adverse event is speed disorder.

Dysgeusia includes dysgeusia or hypogeusia. In some embodiments, the adverse event is dysgeusia. In further embodiments, the adverse event is hypogeusia.

Headache includes sinus headache or a general headache not necessarily associated with sinus. In some embodiments, the adverse event is a headache. In other embodiments, the adverse event is a sinus headache.

“Heart rate increase” or variations thereof includes an increase in heart rate as measured in beats per minute (bpm).

Hypoesthesia includes hypoesthesia, oral hypoesthesia, hypoesthesia teeth, or pharyngeal hypoesthesia. In some embodiments, the adverse event is hypoesthesia. In other embodiments, the adverse event is oral hypoesthesia. In further embodiments, the adverse event is hypoesthesia teeth. In still other embodiments, pharyngeal hypoesthesia.

Lethargy includes fatigue or lethargy. In some embodiments, the adverse event is fatigue. In other embodiments, the adverse event is lethargy.

Nasal discomfort includes nasal crusting, nasal discomfort, nasal dryness, or nasal pruritus, or combinations thereof. In some embodiments, the adverse event is nasal crusting. In other embodiments, the adverse event is nasal discomfort. In further embodiments, the adverse event is nasal dryness. In yet other embodiments, the adverse event is nasal pruritus.

Nausea includes the sensation of an urge to vomit. The nausea may be short-lived or prolonged and/or acute or generalized. Nausea may be accompanied by other symptoms such as diarrhea, gas, constipation, among others. In some embodiments, the nausea may be mild, moderate, or severe. In other embodiments, the nausea is in the absence of vomiting. One of skill in the art would be able to identify if a patient is nauseous.

Sedation, i.e., sleepiness, includes altered state of consciousness, hypersomnia, sedation, or somnolence, or a combination thereof. In some embodiments, the adverse event is an altered state of consciousness. In further embodiments, the adverse event is hypersomnia. In other embodiments, the adverse event is sedation. In yet further embodiments, the adverse event is somnolence.

Tachycardia includes extrasystoles, increase in heart rate, or tachycardia. In some embodiments, the adverse event is extrasystoles. In other embodiments, the adverse event is an increase in heart rate. In further embodiments, the adverse event is tachycardia.

Vertigo includes vertigo or positional vertigo. In some embodiments, the adverse event is vertigo. In other embodiments, the adverse event is positional vertigo.

The term “serious adverse event”, “serious side effect”, and “SAE” as used herein are interchangeable and are defined based on ICH and EU Guidelines on Pharmacovigilance for Medicinal Products for Human Use. The serious adverse event is irrespective of dose. One of skill in the art would understand that a serious adverse event is medically important. In some embodiments, the serious adverse event is an adverse event that results in death. In other embodiments, the serious adverse event is life-threatening, e.g., the subject was at risk of death at the time of the serious adverse event. In further embodiments, the serious adverse event requires inpatient hospitalization or prolongation of existing hospitalization. In yet other embodiments, the serious adverse event results in persistent or significant disability or incapacity. In still further embodiments, the serious adverse event is a congenital anomaly/birth defect. In other embodiments, the serious adverse event is a suspected transmission of any infectious agent via a medicinal product. In further embodiments, the serious adverse event is fainting. In yet other embodiments, the adverse event is a spinning sensation. In still further embodiments, the serious adverse event is anxiety.

The methods have an induction phase of four weeks and treatment sessions. As used herein, an “induction phase” is a period of time that esketamine is initially administered to the patient. In some embodiments, the induction phase is sufficiently long as to achieve a robust, stable reduction of depressive symptoms. For certain indications, such as MDD with suicidal intent: severe MDD or severe MDD when urgent symptom control is necessary, the induction phase constitutes the treatment method, i.e., a maintenance phase is not included.

In the induction period, a patient is administered about 56 mg or about 84 mg of esketamine per induction phase treatment session at a frequency of at least twice a week for 4 weeks. The amount of esketamine administered during the induction phase may be determined by the attending physician. In some embodiments, the effective amount of esketamine administered during the induction phase is about 56 mg. In other embodiments, the effective amount of esketamine administered during the induction phase is about 84 mg.

As discussed below, a nasal spray device is a single-use device that, in certain embodiments, delivers a total of 28 mg of esketamine in two sprays, one spray per nostril. The device may be operated by the patient under the supervision of a healthcare professional or by the healthcare provider. With respect to dosage amounts, one device may be used for a 28 mg dose, two devices for a 56 mg dose, or three devices for an 84 mg dose. As such, the term “treatment session” as used herein refers the period that it takes to administer the prescribed dosage of esketamine (e.g. the 56 or 84 mg). Treatment sessions include induction treatment sessions and maintenance treatment sessions, if applicable. It is also preferable to have a 5-minute interval between the use of each device. Typically, time 0 is defined as the time of administration of the first intranasal spray to one nostril from the first intranasal device. The designated dose of esketamine is administered in a treatment session. For example, the treatment session for 56 mg of esketamine may include 2 sprays from a first device and 2 sprays from a second device. As another example, the treatment session for 84 mg of esketamine may include 2 sprays from a first device (1 spray to each nostril), 2 sprays from a second device (1 spray to each nostril), and 2 sprays from a third device (1 spray to each nostril). However, more devices may be as needed, e.g., if a device fails to operate properly and additional devices are required to administer the required dosage or amount of esketamine. The treatment session typically begins when the first spray is administered to one nostril from the first device. The treatment session ends when the last spray is administered to a nostril from the last device.

The term “twice weekly” as used herein refers to a frequency that is two times in a weekly (7-day) period. For example, “twice weekly” may refer herein to the administration of esketamine. In some embodiments, twice weekly refers to a frequency that is day 1 and day 2 of a week. In other embodiments, twice weekly refers to a frequency that is day 1 and day 3 of a week. In further embodiments, twice weekly refers to a frequency that is day 1 and day 4 of a week. In still other embodiments, twice weekly refers to a frequency that is day 1 and day 5 of the week. The “day 1” may be any day of the week, including, Sunday, Monday, Tuesday, Wednesday, Thursday, Friday, or Saturday. Typically, with respect to administration of esketamine, twice weekly refers to a frequency that is day 1 and day 4 of a week. To the extent there is a mis-dose, the dose may be taken as soon as possible thereafter and the prescribed regimen thereafter continued.

The term “once weekly” as used herein refers to a frequency that is one time in a weekly (7-day) period. For example, “once weekly” may refer herein to the administration of esketamine. In some embodiments, once weekly refers to a frequency that is day 1 of a week. In other embodiments, once weekly refers to a frequency that is day 2 of a week. In further embodiments, once weekly refers to a frequency that is day 3 of a week. In still other embodiments, once weekly refers to a frequency that is day 4 of the week. In further embodiments, once weekly refers to a frequency that is day 5 of the week. In other embodiments, once weekly refers to a frequency that is day 6 of the week. In yet other embodiments, once weekly refers to a frequency that is day 7 of the week. The “day 1” may be any day of the week, including, Sunday, Monday, Tuesday, Wednesday, Thursday, Friday, or Saturday. To the extent there is a mis-dose, the dose may be taken as soon as possible thereafter and the prescribed regimen thereafter continued.

At any stage during the treatment session, the patient's response to the treatment session may be assessed using techniques described herein. This assessment may be performed until the patient is considered by one skilled in the art ready to leave the testing site. Thus, such assessments may be performed before, during, or after each treatment session. Preferably, the patient's response is assessed by determining the number, if any, of adverse events, including serious adverse events, experienced by the patient. In cases where the patient experiences a serious adverse event, the patient does not become a qualified patient. If the patient does not experience a severe adverse event and does not experience any of a clinically meaningful blood pressure increase, clinically meaningful heart rate increase, moderate or greater levels of dissociation, moderate or greater levels of sedation or disorientation, and/or moderate or greater levels of nausea without vomiting, for at least two consecutive treatment sessions after being monitored for at least 90 minutes (e.g., 2 hours) following each treatment session, the patient becomes a qualified patient. In other embodiments, a qualified patient is one that does not does not experience a severe adverse event and does not experience any of a clinically meaningful blood pressure increase, moderate or greater levels of dissociation, moderate or greater levels of sedation, moderate or greater levels of dizziness, and/or moderate or greater levels of vertigo, for at least two consecutive treatment sessions after being monitored for at least 90 minutes (e.g., 2 hours) following each treatment session. In yet other embodiments, a qualified patient is one that does not experience a severe adverse event and does not experience any of a clinically meaningful blood pressure increase, clinically meaningful heart rate increase, moderate or greater levels of dissociation, and/or moderate or greater levels of sedation, for at least two consecutive treatment sessions after being monitored for at least 90 minutes (e.g., 2 hours) following each treatment session.

Prior to being designated a qualified patient, the patient is desirably assessed at regular intervals in a post-treatment session monitoring period of at least 90 minutes. However, assessments may be made as needed, including instances where the patient may be in distress. Desirably, the patient's blood pressure is taken, and the patient evaluated for dissociation symptoms, sedation symptoms, dizziness symptoms, nausea, and/or vertigo symptoms. In some embodiments, the patient is assessed at least about 5 minute intervals after the treatment session is completed. In other embodiments, the patient is assessed at about 5, about 10, about 15, about 20, about 25, or about 30 minutes intervals. In further embodiments, the patient is assessed at intervals of about 5 to about 30, about 5 to about 25, about 5 to about 20, about 5 to about 15, about 5 to about 10, about 10 to about 30, about 10 to about 25, about 10 to about 20, about 10 to about 15, about 15 to about 30, about 15 to about 25, about 15 to about 10, about 20 to about 30, about 20 to about 25, or about 25 to about 30 minute intervals. Preferably, the patient is administered at about 15 minute intervals. The assessment continues as long as the patient is exhibiting adverse events. In some embodiments, the assessment is performed for at least about 90 minutes. In other embodiments, the assessment is performed for about 90 to about 180 minutes, about 90 to about 170, about 90 to about 160, about 90 to about 150, about 90 to about 140, about 90 to about 130, about 90 to about 130, about 90 to about 120, about 90 to about 110, about 90 to about 100, about 100 to about 180, about 100 to about 170, about 100 to about 160, about 100 to about 160, about 100 to about 150, about 100 to about 140, about 100 to about 130, about 100 to about 120, about 100 to about 110, about 110 to about 180, about 110 to about 170, about 110 to about 160, about 110 to about 150, about 110 to about 140, about 110 to about 130, about 110 to about 120, about 120 to about 180, about 120 to about 170, about 120 to about 160, about 120 to about 150, about 120 to about 140, about 120 to about 130, about 130 to about 180, about 130 to about 170, about 130 to about 160, about 130 to about 150, about 130 to about 140, about 140 to about 180, about 140 to about 170, about 140 to about 160, about 140 to about 150, about 150 to about 180, about 150 to about 170, about 150 to about 160, about 160 to about 180, about 160 to about 170, or about 170 to about 180 minutes. Preferably, the assessment is performed for about 90 to about 120 minutes. Following the monitoring period, the patient is typically released from the testing site based on clinical judgment.

It also is desirable that the patient tolerated esketamine and the associated side effects/adverse events without medical intervention, during all treatment sessions and prior to becoming a qualified patient. Thus, a patient does not require any emergency treatment as a result of esketamine administration before becoming a qualified patient.

Once the patient is designated a qualified patient, the patient is still monitored after a treatment session. However, the post treatment monitoring session may be shorter compared to the post treatment session monitoring period used before patient qualification. In some embodiments, the qualified patient is assessed at regular intervals following the treatment session. In some embodiments, the patient is assessed at least about 5 minute intervals after the treatment session is completed. In other embodiments, the patient is assessed at about 5, about 10, about 15, about 20, about 25, or about 30 minutes intervals. In further embodiments, the patient is assessed at intervals of about 5 to about 30, about 5 to about 25, about 5 to about 20, about 5 to about 15, about 5 to about 10, about 10 to about 30, about 10 to about 25, about 10 to about 20, about 10 to about 15, about 15 to about 30, about 15 to about 25, about 15 to about 10, about 20 to about 30, about 20 to about 25, or about 25 to about 30 minute intervals. The assessment continues for a period of time that is shorter than that required before patient qualification. In some embodiments, the assessment is performed for less than about 90 minutes. In other embodiments, the assessment is performed for about 30 to less than about 90, about 30 to about 80, about 30 to about 70, about 30 to about 60, about 30 to about 50, about 30 to about 40, about 40 to about 90, about 40 to about 80, about 40 to about 70, about 40 to about 60, about 40 to about 50, about 40 to about 45, about 50 to about 90, about 50 to about 80, about 50 to about 70, about 50 to about 60, about 60 to about 90, about 60 to about 80, about 60 to about 70, about 70 to about 90, about 70 to about 80, or about 80 to about less than 90 minutes. In certain embodiments, a qualified post-treatment session monitoring period is at least 60 minutes. In further embodiments, a qualified post-treatment monitoring period is from about 60 minutes to less than 90) minutes. In other embodiments the post-treatment session monitoring period is about 60) minutes.

After the patient is deemed stable after the post-treatment session monitoring period, the patient is free to leave the clinic or medical setting where esketamine was administered. However, in order to discharge the patient, the patient should be monitored and/or required to contact a health care professional and/or submit a patient monitoring form. In one embodiment, a caregiver or responsible adult accompanies the patient when the post-treatment session monitoring period is less than 2 hours. Preferably, the caregiver or responsible adult is present during one or both of the patient's treatment session and post-treatment session monitoring period. In addition to ensuring that the patient is safely transported away from the clinic, the caregiver or responsible adult may assist the patient in monitoring for any adverse effects resulting from esketamine after leaving the clinic.

The term “caregiver” as used herein refers to an adult that is legally responsible for the care and well-being of the patient. Thus, the caregiver may be a family member (e.g., parent, sibling, child, etc.) or a legal guardian. The term “responsible adult” refers to an adult that is physically and mentally capable of assisting the patient, but who is not legally responsible for the care of the patient. In some embodiments, the caregiver or responsible adult is present during the treatment session, is present during the post-treatment session monitoring period, assists the patient away from the clinic (e.g., to home or place of residence), monitors the patient for a predetermined amount of time after leaving the clinic, assists the patient in remote interactions with the clinician, or any combinations thereof. Preferably, the caregiver or responsible adult provides his/her provide their information (e.g., name, relationship, contact information) to the clinician prior to the patient's treatment with esketamine.

After leaving the clinic, the caregiver or responsible adult may participate in one or more follow-up assessments between the patient and clinician to assess the patient's clinical status if the post-treatment session monitoring period is less than 2 hours. The term “follow-up assessment” as used herein refers to interactions between the patient and clinician to monitor the patient's response to esketamine. During these follow-up assessments, the clinician assess the physical and mental state of the patient. In some embodiments, the clinician may determine how many and which adverse events the patient experienced after the post-treatment session monitoring period. Preferably, the clinician will document any adverse events experienced by the patient. In certain embodiments, the clinician will document any events such as sedation, dissociation, and/or serious adverse events as determined by the clinician that are experienced by the patient. In other embodiments, the clinician determines if the patient experienced a new onset of sedation and/or dissociation after the post-treatment session monitoring period. Adverse events deemed of particular importance are noted by the clinician and documented (e.g., on relevant monitoring forms). In some embodiments, the caretaker or responsible adult may arrange any follow-up assessments with the clinician. In further embodiments, the caretaker or responsible adult may participate in the follow-up assessments between the patient and clinician.

Desirably, the first follow-up assessment is the same day of the treatment session. In some embodiments, this is the only follow-up assessment conducted between the clinician and the patient until the next treatment session with esketamine. However, if the clinician determines that further monitoring is required, further follow-up assessments are conducted. In some embodiments, the follow-up assessments are conducted on an hourly or daily basis. In a particular embodiment, the follow-assessments are continued daily after the initial follow-up assessment, e.g., follow-up sessions are continued daily for 1, 2, 3, 4, 5, 6, 7, 8, 9, 10, 11, 12, 13, 14, or greater days following the initial follow-up assessment. If clinically indicated, one of skill in the art would be able to determine how often and many follow-up assessments are required after the initial follow-up assessment. In particular embodiments, follow-up sessions are continued daily for 1 day following the initial follow-up assessment. In other embodiments, follow-up sessions are continued daily for 2 days following the initial follow-up assessment. In further embodiments, follow-up sessions are continued daily for 3 days following the initial follow-up assessment. In yet other embodiments, follow-up sessions are continued daily for 4 days following the initial follow-up assessment. In still further embodiments, follow-up sessions are continued daily for 5 days following the initial follow-up assessment. In other embodiments, follow-up sessions are continued daily for 6 days following the initial follow-up assessment. In further embodiments, follow-up sessions are continued daily for 7 days following the initial follow-up assessment.

Interactions between the patient and clinician (optionally with the caregiver or responsible adult) may be conducted using a variety of techniques available to the patient and attending physician. The interactions may be performed to conduct patient assessments, including monitoring the patient's physical health. In some embodiments, interactions between the physician and patient are in person, i.e., at the clinic where esketamine is administered. In other embodiments, interactions between the physician and patient are remote, e.g., by telephone (e.g., oral and/or visual). In further embodiments, interactions between the physician and patient are done remotely using other electronic means such as video conferencing, among others. In some embodiments, the attending physician contacts the patient at least about 2 hours on the same day after leaving the site of esketamine administration, i.e., about 2 hours after the post-treatment session monitoring period.

In order to facilitate compliance and gauge patient tolerance to esketamine, it may be necessary to complete one or more forms to track the patient's general health, include measurable parameters such as heart rate and blood pressure, noting of adverse events of any significance, among others.

The administration may further comprise a maintenance phase that follows the induction phase. During the maintenance phase, esketamine is administered at a frequency of once weekly for the first four weeks of the maintenance phase. Thereafter, the dosing frequency may be adjusted as determined by the attending physician (e.g. based on tolerability). In some embodiments, the dosing frequency is once weekly, i.e., no more than one treatment session per week. In other embodiments, the dosing frequency is adjusted to once every other week. In some embodiments, the amount of esketamine administered during the maintenance phase is about 56 mg. In other embodiments, the effective amount of esketamine administered during the maintenance phase is about 84 mg. Typically, at the time of the post-treatment session monitoring period, the patient is receiving no more than one treatment session per week, e.g., typically a patient that is in a maintenance phase of the treatment.

At any stage during one or more of an induction phase or maintenance phase, the patient's response to the treatment is assessed using techniques described herein. This assessment may be performed until the patient is considered by one skilled in the art to have achieved a suitable response to the treatment regimen. In some embodiments, the assessment includes a determination of the patient's mental state using techniques known to those in the art and described herein. In other embodiments, the assessment includes a determination of the patient's physical status including, without limitation, heartrate, heart rhythm, vision, hearing, blood pressure, breathing, among others. In some embodiments, blood pressure measurements are taken at intervals of about 40 minutes.

The patient is monitored after at least the first two treatment sessions in a monitoring period of at least 90 minutes prior to becoming a qualified patient. In some embodiments the pre-qualifying monitoring period is about 120 minutes. In some embodiments, the patient is monitored after about 2, about 3, about 4, about 5, about 6, about 7, about 8, about 9, about 10), about 11, about 12, about 13, about 14, about 15, about 16, about 17, about 18, about 19, or about 20 treatment sessions, i.e., about 1 to about 10 weeks, prior to becoming a qualified patient. In other embodiments, the patient is monitored for about 2 treatment sessions, i.e., about 1 week, prior to becoming a qualified patient. In further embodiments, the patient is monitored for about 4 treatment sessions, i.e., about 2 weeks prior to becoming a qualified patient. In other embodiments, the patient is monitored for about 8 treatment sessions, i.e., about 4 weeks prior to becoming a qualified patient (i.e., the induction phase). In further embodiments, the patient is monitored for about 8 treatment sessions, i.e., about 8 weeks prior to becoming a qualified patient (i.e., 4 weeks of the induction phase). In yet another embodiment, the patient is monitored for about 12 treatment session, i.e. about 6 weeks prior to becoming a qualified patient. In yet further embodiments, the patient is monitored for about 16 treatment sessions, i.e., about 8 weeks prior to becoming a qualified patient.

A number of physical parameters may be measured when assessing the patient's physical status. In some embodiments, the attending physical may determine the absence or extent of an adverse event caused by treatment session. In general, the adverse event is any severe adverse event requiring medical intervention. The term “adverse event” as used herein refers to a physical response after the treatment session to esketamine. In some embodiments, the adverse event is one or more of dissociation, disorientation, increased blood pressure, increased heart rate, nausea (without vomiting), vomiting, sedation, feeling drunk, vertigo, hypesthesia, anxiety, dizziness, or lethargy. In other embodiments, the adverse event is dissociation. In further embodiments, the adverse event is increased blood pressure. In yet further embodiments, the adverse event is increased heart rate. In yet other embodiments, the adverse event is nausea. In still further embodiments, the adverse event is vomiting. In other embodiments, the adverse event is moderate or greater levels of nausea without vomiting. In yet other embodiments, the adverse event is sedation. In further embodiments, the adverse event is feeling drunk. In still other embodiments, the adverse event is vertigo. In yet further embodiments, the adverse event is hypesthesia. In other embodiments, the adverse event is anxiety. In further embodiments, the adverse event is dizziness. In yet other embodiments, the adverse event is lethargy. In other embodiments, the adverse event is a blood pressure increase, heart rate increase, dissociation, moderate or greater levels of nausea without vomiting, sedation, dizziness, and/or vertigo, among others.

In other embodiments, before being a qualified patient for a shorter post-treatment session monitoring period (e.g. less than 90 minutes) in any next treatment session, the patient is monitored after at least the first two treatment sessions for a period of at least 90 minutes. If the patient does not experience a serious adverse event and does not experience any of the following adverse events: clinically meaningful blood pressure increase, clinically meaningful heart rate increase, moderate or greater levels of dissociation, moderate or greater levels of sedation, moderate or greater levels of dizziness, moderate or greater levels of nausea without vomiting, and/or moderate or greater levels of vertigo, following any two consecutive treatment sessions, the patient becomes qualified for a shorter post-treatment session monitoring period (e.g. less than 90) minutes, preferably about 60) minutes) in any next treatment session. As noted herein, the pre-qualification monitoring period can include more than the at least first two treatment sessions, such as the first eight treatment sessions, without the patient experiencing a serious adverse event and any of the following adverse events: clinically meaningful blood pressure increase, clinically meaningful heart rate increase, moderate or greater levels of dissociation, moderate or greater levels of sedation, moderate or greater levels of dizziness, moderate or greater levels of nausea without vomiting and/or moderate or greater levels of vertigo. In any event, typically the adverse event conditions noted above should be met in pre-qualification monitoring periods following two consecutive treatment sessions. The more treatments sessions without encountering the enumerated adverse events provides even a greater likelihood that the patient will not experience them in any next treatment session. Following a post-treatment session monitoring period of less than 90 minutes, the qualified patient is typically released after a healthcare professional determines that the qualified patient is clinically stable and discharge ready.

If at any time during esketamine treatment, i.e., during a treatment session or afterward, the patient experiences a serious adverse event or experience any of the following adverse events: clinically meaningful blood pressure increase, clinically meaningful heart rate increase, moderate or greater levels of disorientation, moderate or greater levels of sedation, and/or moderate or greater levels of nausea without vomiting, or any combination thereof, the patient is disqualified from reduced monitoring session. The disqualified patient, thus, is required to stay the full 2 hours or longer during the post-treatment session monitoring period. In a particular embodiment, the patient is prohibited from future post-treatment session monitoring periods that are less than 2 hours. In other embodiments, the patient may attempt to qualify for future post-treatment session monitoring periods that are less than 2 hours as determined by the attending clinician. For example, if the patient does not show any disqualifying symptoms for 4, 6, 8, 10, or 12 treatment sessions, the physician at his discretion may requalify the patient for a monitoring period of less than 2 hours as described herein.

As used herein, unless otherwise noted, the term “clinically” (used independently or to modify the term “meaningful”) shall mean that a result is meaningful that would be sufficient to standards of the U.S. Food and Drug Administration or similar study for market authorization by EMEA. A clinically meaningful blood pressure increase and/or clinically meaningful heart rate increase can be determined by clinical judgment. In some embodiments, the blood pressure increase may be an increase systolic, diastolic, or a combination thereof. For example, a systolic blood pressure increase of >20 mmHg than baseline and/or a diastolic blood pressure increase of ≥15 mmHg than baseline can be deemed clinically meaningful. In other examples, a systolic blood pressure increase of >40) mmHg than baseline and/or a diastolic blood pressure increase of ≥25 mmHg than baseline can be deemed clinically meaningful. In further examples, a systolic blood pressure of ≥180 mmHg and/or a diastolic blood pressure of ≥110 mmHg can be deemed clinically meaningful. Further, a heart rate increase of ≥20 bpm than baseline and/or heart rate of ≥ 100 bpm can be deemed clinically meaningful. In other examples, a heart rate increase of ≥ 15 bpm than baseline can be deemed clinically meaningful.

One of skill in the art would readily be able to measure a patient's adverse events from experience and one or more tools utilized to make such assessments. For example, the Clinician-Administered Dissociative States Scale (CADSS) is an technique utilized to measure present-state dissociative symptoms, and, thus, may be utilized to assess treatment-emergent dissociative symptoms. The CADSS consists of 23 subjective items, divided into 3 components: Depersonalization (Items 3 to 7, 20, and 23), derealization (Items 1, 2, 8 to 13, 16 to 19, and 21) and amnesia (Items 14, 15, and 22). Participant's responses are coded on a 5-point scale (0=not at all through to 4=extremely). Thus, higher CADSS values represent a worse dissociative state, whereas lower CADSS values are indicative of a mild dissociative state.

For measuring treatment session induced sedation, one of skill in the art may use the Modified Observer's Assessment of Alertness/Sedation Scale (MOAA/S). The MOAA/S scores range from (=no response to painful stimulus (corresponds to ASA continuum for general anesthesia) to 5=readily responds to name spoken in normal tone (awake: corresponds to ASA continuum for minimal sedation). On each intranasal dosing day, the MOAA/S will be performed every 15 minutes from predose to t=+1.5 hours postdose. If the score is ≤3 at any time during the 1.5 hour postdose interval, the MOAA/S will be performed every 5 minutes until a score of 4 is reached (at which point a frequency of every 15 minutes can be resumed until t=+1.5 hours post dose). However, if a subject does not have a score of at least 5 at t=+1.5 hours postdose, they should continue to be monitored. For subjects with a score of 4, the assessment should be repeated every 15 minutes. And for subjects with a score of ≤3, the assessment should be repeated every 5 minutes until the score. In some embodiments, the results of the MOAA/S may be correlated to levels of sedation defined by the American Society of Anesthesiologists (ASA) continuum.

The Clinical Global Assessment of Discharge Readiness (CGADR) may also be utilized, together with other parameters, to measure the subject's current clinical status and is the clinician's assessment of the readiness to be discharged from the study site. The clinician answers “Yes” or “No” to the question “Is the subject considered ready to be discharged based on their overall clinical status (e.g., sedation, blood pressure, and other adverse events)?” On each intranasal dosing day, the CGADR will be performed at 1 hour or 1.5 hours postdose: if the response is not “Yes” at 1.5 hours postdose, the assessment will be repeated every 15 minutes until a “Yes” response is achieved or until the subject is referred for appropriate medical care, if clinically indicated. If the patient is a qualified patient, the CGADR (or a similar discharge readiness assessment) will be performed about 15 minutes before the end of the post treatment session monitoring period response is not “Yes” at 45 minutes postdose (post treatment session), the assessment will be repeated every 15 minutes until a “Yes” response is achieved or until the subject is referred for appropriate medical care, if clinically indicated.

For adverse events, including those that do not have a standard way to assess severity, one of skill in the art may make an assessment of severity grade using the general categorical descriptors of “mild”, “moderate”, or “severe”. The grade “mild” is correlated to a patient having awareness of symptoms that are easily tolerated, causing minimal discomfort and not interfering with every day activities. The grade “moderation” or “moderate” are used for a patient with sufficient discomfort to cause interference with normal activity. The grade “severe” is used for a patient having extreme distress, causing significant impairment of functioning or incapacitation, thereby preventing normal everyday activities.

Typically, a qualified patient does not require medical intervention and/or does not require medical observation that is related to resolving an adverse event during pre-qualification post-treatment session monitoring periods. The term “medical intervention” as used herein refers to the need for a medical professional to care for a patient. Medical intervention may include one or more of a blood test, breathing assistance, cardiac assistance, administration of medication to reduce an adverse event or symptoms of an adverse event, emergency care, among others. Similarly, the term “medical observation” as used herein refers to close monitoring of the patient by a medical professional. The monitoring may be visual, consist of interactions with the patient, optionally gauging patient responses to questions, or may include tests such as heartrate, heart rhythm, vision, hearing, blood pressure, breathing, among others in an effort to determine if medical intervention is needed.

As disclosed herein, qualified patients have a lower risk of developing any adverse event in post-treatment session monitoring periods, and, thus, do not require the same amount of time to monitor. In some embodiments, a qualified patient requires no medical intervention in any prior treatment sessions. In other embodiments, a qualified patient exhibits no clinically relevant or meaningful side effects of concern per clinical judgment. As disclosed herein, conventional post-treatment monitoring periods require about 1.5 and 2 hours of monitoring by a medical professional. This period of time permits the medical professional to evaluate the adverse events to determine if mild or no adverse events are encountered. Doing so also provides a greater confidence of no significant adverse events from occurring post dose and allows for identification of qualified patients that require shorter post-treatment session monitoring periods. It should be noted that to the extent the patient has a dosage adjustment, e.g., 56 mg to 84 mg, although the effect of dosing on AE is small it may be advisable to requalified for the shorter post-treatment session monitoring period. Typically, the elderly do not qualify for a shorter post-treatment session monitoring period because they are more likely to exhibit adverse events related to blood pressure changes as well as the difficulty in predicting blood pressure changes in the elderly.

One of skill in the art would be able to determine if an adverse event has been “resolved” using skill in the art. In some embodiments, an adverse event is resolved if all or substantially all of the symptoms regarding the adverse event dissipate. In other embodiments, an adverse event is resolved if the patient can function normally, i.e., function after esketamine administration as the patient functioned before esketamine administration.

As disclosed herein, typically, it takes two consecutive treatment sessions without the patient experiencing a serious adverse event and any of the following adverse events: clinically meaningful blood pressure increase, clinically meaningful heart rate increase, moderate or greater levels of dissociation, moderate or greater levels of sedation, moderate or greater levels of dizziness, moderate or greater levels of nausea without vomiting, and/or moderate or greater levels of vertigo. In some embodiments, the patient becomes a qualified patient after at least the first three consecutive treatment sessions. In some embodiments, the patient is qualified after at least about 3, about 4, about 5, about 6, about 7, about 8, about 9, about 10, about 11, about 12, or more treatment sessions. In some embodiments, the patient is qualified after about 4 treatment sessions. In other embodiments, the patient is qualified after about 8 treatment sessions. In further embodiments, the patient is qualified after about 3 to about 12, about 3 to about 11, about 3 to about 10, about 3 to about 9, about 3 to about 8, about 3 to about 7, about 3 to about 8, about 3 to about 7, about 3 to about 6, about 3 to about 5, about 3 to about 4, about 4 to about 12, about 4 to about 11, about 4 to about 10, about 4 to about 9, about 4 to about 8, about 4 to about 7, about 4 to about 8, about 4 to about 7, about 4 to about 6, about 4 to about 5, about 5 to about 12, about 5 to about 11, about 5 to about 10, about 5 to about 9, about 5 to about 8, about 5 to about 7, about 5 to about 6, about 6 to about 12, about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 9 to about 11, about 9 to about 10, or about 10 to about 11 weeks.

Some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “esketamine” shall mean the(S)-enantiomer of ketamine, i.e., a compound of formula (I):

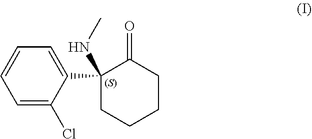

also known as(S)-2-(2-chlorophenyl)-2-(methylamino)cyclohexanone. “Esketamine” also includes it salts, e.g., a chloride salt such as the hydrochloride salt, of the(S)-enantiomer of ketamine, i.e., a compound of formula (II):

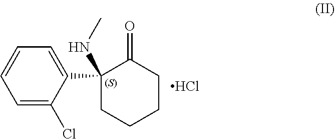

also known as(S)-2-(2-chlorophenyl)-2-(methylamino)cyclohexanone hydrochloride. In some embodiments, the esketamine is substantially free of the (R)-enantiomer of ketamine, i.e. a compound of formula (III):

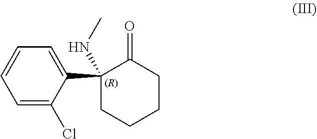

In other embodiments, the esketamine contains less than about 10% by weight, based on the weight of the esketamine sample, of the (R)-enantiomer of ketamine. In further embodiments, the esketamine contains less than about 10, 9, 8, 7, 6, 5, 4, 3, 2, 1, 0.5, 0.1, 0.005, or 0.001% by weight, based on the weight of the esketamine sample, of the (R)-enantiomer of ketamine. In yet other embodiments, the esketamine contains about 0.001 to about 10% by weight, based on the weight of the esketamine sample, of the (R)-enantiomer of ketamine. In still further embodiments, the esketamine contains about 0.001 to about 10%, about 0.001 to about 5%, about 0.001 to about 1, about 0.001 to about 0.5, about 0.001 to about 0.1, about 0.1 to about 5, about 0.1 to about 1, about 0.1 to about 5, or about 0.5 to about 5% by weight, based on the weight of the esketamine sample, of the (R)-enantiomer of ketamine.

The term “esketamine” may also include other pharmaceutically acceptable salts thereof, which may readily be selected by those skilled in the art. A “pharmaceutically acceptable salt” is intended to mean a salt of esketamine that is non-toxic, biologically tolerable, or otherwise biologically suitable for administration to the subject. See, generally, G. S. Paulekuhn, “Trends in Active Pharmaceutical Ingredient Salt Selection based on Analysis of the Orange Book Database”, J. Med. Chem., 2007, 50:6665-72, S. M. Berge, “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for administration to patients without undue toxicity, irritation, or allergic response.

Examples of other pharmaceutically acceptable salts include sulfates, pyrosulfates, bisulfates, sulfites, bisulfites, phosphates, monohydrogen-phosphates, dihydrogenphosphates, metaphosphates, pyrophosphates, bromides (such as hydrobromides), iodides (such as hydroiodides), acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxy benzoates, methoxy benzoates, phthalates, sulfonates, xylenesulfonates, phenylacetates, phenylpropionates, phenylbutyrates, citrates, lactates, γ-hydroxy butyrates, glycolates, tartrates, methane-sulfonates, propanesulfonates, naphthalene-1-sulfonates, naphthalene-2-sulfonates, and mandelates. In particular, the salt of esketamine is a hydrochloride salt.

In certain embodiments, the esketamine is administered intranasally. In other embodiments, the esketamine is administered intranasally as its corresponding hydrochloride salt. In further embodiments, the esketamine is administered intranasally as its corresponding hydrochloride salt in an 16.14% weight/volume solution (equivalent to 14% weight/volume of esketamine base).

In certain embodiments, the esketamine is administered intranasally as a solution comprising 161.4 mg/mL of esketamine hydrochloride (equivalent to 140 mg/ml of esketamine base), 0.12 mg/mL of ethylenediaminetetraacetic acid (EDTA) and 1.5 mg/mL citric acid, at a pH of 4.5 in water. In other embodiments, the esketamine is administered intranasally, wherein the intranasal delivery administers 100 μL of a solution comprising 161.4 mg/mL of esketamine hydrochloride (equivalent to 140 mg/mL of esketamine base), 0.12 mg/mL of ethylenediaminetetraacetic acid (EDTA) and 1.5 mg/mL citric acid, at a pH of 4.5 in water. In further embodiments, the esketamine is delivered intranasally using a nasal spray pump, wherein the pump delivers 100 μL of a solution comprising 161.4 mg/ml of esketamine hydrochloride (equivalent to 140 mg/mL of esketamine base), 0.12 mg/ml of ethylenediaminetetraacetic acid (EDTA) and 1.5 mg/mL citric acid, at a pH of 4.5 in water.

In general, a single pump from a nasal spray device may be configured to deliver about 50 μL to about 200 μL of an esketamine solution to a nostril of the subject, including about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, and about 200 μL. Accordingly, two pumps deliver about 100 μL to about 400 μL to the subject.

In certain embodiments, a patient in need of treatment with a therapeutically effective amount of esketamine, is a patient suffering from an episode of depression (e.g., major depressive disorder). In other embodiments, a patient in need thereof is suffering from an episode of depression (e.g., major depressive disorder), wherein the episode of depression (e.g., major depressive disorder) has not responded to treatment with at least two oral antidepressants (i.e. the patient has not responded to treatment with at least two oral antidepressants).

At the end of the induction phase, the treating physician may evaluate the patient to optimize the dosing amount and frequency for any subsequent administration phases such as the “maintenance phase”. It is anticipated that the intranasal treatment frequency during the subsequent administration such as the maintenance phase will be reduced from that in the induction phase (at least twice weekly) to once weekly dosing for at least 4 weeks. In some embodiments, the subsequent administration such as the maintenance phase is at least about 4 weeks, about 5 weeks, about 6 weeks, about 7 weeks, about 8 weeks, about 9 week, about 10 weeks, about 11 weeks, about 12 weeks, about 13 weeks, about 17 weeks, about 18 weeks, about 19 weeks, about 20 weeks, about 6 months, about 7 months, about 8 months, about 9 months, about 10 months, about 11 months, 1 year, or about 2 years. In some embodiments, the administration of the esketamine during the maintenance phase is at least six months. In other embodiments, the administration of the esketamine during the maintenance phase is at least one year. In further embodiments, the frequency of administration during the maintenance phase is once every week or once every two weeks, or a combination thereof. In yet other embodiments, the dosing frequency and effective amount of esketamine during the maintenance phase is the minimum frequency and amount required to treat the depression.

The subsequent administration, such as in a maintenance period, may include longer periods of time depending on the patient's condition. In some embodiments, those longer periods may be at least about 3 years, about 4 years, about 5 years, about 6 years, about 7 years, about 8 years, about 9 years, about 10 years, or more than about 10 years, including indefinitely. For example, for patients diagnosed with TRD, treatment may be indefinite. In other embodiments, the treatment frequency is reduced to biweekly. In further embodiments, the treatment frequency is reduced to every three weeks. In yet other embodiments, the treatment frequency is reduced to monthly. The patients will be maintained on schedule until the patient achieves remission, maintains a response, or fails treatment. If the patient achieves remission or maintains a response with the once a week treatment for at least 4 weeks, the frequency of intranasal treatment sessions may be decreased to a maintenance dose of once every other week based on the severity of depressive symptoms and for some patient populations the frequency of treatment may be reduced to about once every three or four weeks as discussed above.

One skilled in the art will recognize that the maintenance phase described herein may continue until further treatment is not required and as indicated by, for example, prolonged remission of the depression (including for example, the remission of one or more symptoms associated with depression), social and/or occupational functional improvement to normal or premorbid levels, or other known measures of depression.

The amount of esketamine administered during the maintenance phase is the amount which maintains a pharmacodynamic steady state of esketamine attained in the induction phase. In some embodiments, about 56 mg or about 84 mg of esketamine is administered to the patient during the maintenance phase. For example, the dosage of certain patients taking about 56 mg esketamine may be increased to about 84 mg if depressed symptoms begin to worsen, thereby stabilizing the patient. Alternatively or additionally, if the patient is being dosed every other week and their symptoms begin to worsen, esketamine can be administered once per week to maintain response during the maintenance phase. Again, at any time during the maintenance phase the patient's response may be reassessed.

In the event that one or more (e.g., two) doses of esketamine in any of the phases described herein are missed, the next dose is scheduled when possible based on the dosing frequency regimen. If more than two doses are missed, per clinical judgement, adjustment of the dose or frequency of esketamine may be required.

Also encompassed by the methods described herein include adjunctive treatment with a therapeutically effective amount of one or more antidepressants. Desirably, the adjunctive therapy is during the induction phase, maintenance phase, or both. In some embodiments, the adjunctive therapy is during the induction phase. In other embodiments, the adjunctive therapy is during maintenance phase. In further embodiments, the adjunctive therapy is during the induction and maintenance phases. In certain embodiments, esketamine may be administered in combination with one or more antidepressants, as herein described, preferably in combination with one to three antidepressants, more preferably in combination with one to two antidepressants. In other embodiments, esketamine may be administered in combination with one or more antidepressants, and further in combination with one or more atypical antipsychotics, herein described. The antidepressant should be at least about 2 hours after the treatment session described herein is performed. Such an administration is desirable in an effort to minimize side effects. In some embodiments, the antidepressant is administered at least about 3, about 4, about 5, about 6, about 7, or about 8 hours after the treatment session. In other embodiments, the antidepressant is administered about 3 to about 8, 3 to about 7, 3 to about 6, 3 to about 5, 3 to about 4, 4 to about 8, 4 to about 7, 4 to about 6, 4 to about 5, about 5 to about 8, about 5 to about 7, about 5 to about 6, about 6 to about 8, about 6 to about 7, or about 7 to about hours.

The timing for adjunctive therapy is determined by the attending physician. In some embodiments, the adjunctive therapy is at least 3 hours after an induction phase or maintenance phase treatment session. In other embodiments, the adjunctive therapy is at least about 3, about 4, about 5, about 6, about 7, about 8, about 9, about 10, about 11, about 12, about 18, or about 24 hours after an induction phase treatment session. In further embodiments, the adjunctive therapy is about 3 to about 12, about 3 to about 11, about 3 to about 10, about 3 to about 9), about 3 to about 8, about 3 to about 7, about 3 to about 6, about 3 to about 5, about 3 to about 4, about 4 to about 12, about 4 to about 11, about 4 to about 10, about 4 to about 9, about 4 to about 8, about 4 to about 7, about 4 to about 6, about 4 to about 5, about 5 to about 12, about 5 to about 11, about 5 to about 10, about 5 to about 9, about 5 to about 8, about 5 to about 7, about 5 to about 6, about 6 to about 12, about 6 to about 11, about 6 to about 10, about 6 to about 9, about 6 to about 8, about 6 to about 7, about 7 to about 12, about 7 to about 11, about 7 to about 10, about 7 to about 9, about 7 to about 7, about 8 to about 12, about 8 to about 11, about 8 to about 10, about 8 to about 9, about 9 to about 12, about 9) to about 11, about 9 to about 10, about 10 to about 12, about 10 to about 11, about 11 to about 12 hours after an induction phase treatment session. In other embodiment, the adjunctive therapy is at least 3, 4, 5, 6, 7, 8, 9, 10, 11, 12, 18, or 24 hours after a maintenance phase treatment session. In further embodiments, the adjunctive therapy is about 3 to about 12, about 3 to about 11, about 3 to about 10, about 3 to about 9, about 3 to about 8, about 3 to about 7, about 3 to about 6, about 3 to about 5, about 3 to about 4, about 4 to about 12, about 4 to about 11, about 4 to about 10, about 4 to about 9, about 4 to about 8, about 4 to about 7, about 4 to about 6, about 4 to about 5, about 5 to about 12, about 5 to about 11, about 5 to about 10, about 5 to about 9, about 5 to about 8, about 5 to about 7, about 5 to about 6, about 6 to about 12, about 6 to about 11, about 6 to about 10, about 6 to about 9, about 6 to about 8, about 6 to about 7, about 7 to about 12, about 7 to about 11, about 7 to about 10, about 7 to about 9, about 7 to about 7, about 8 to about 12, about 8 to about 11, about 8 to about 10, about 8 to about 9, about 9 to about 12, about 9 to about 11, about 9 to about 10, about 10 to about 12, about 10 to about 11, about 11 to about 12 hours after a maintenance phase treatment session.

As used herein, the term “adjunctive treatment” and “adjunctive therapy” shall mean treatment of a patient in need thereof by administering esketamine in combination with one or more antidepressant(s), wherein the esketamine and the antidepressant(s) are administered by any suitable means. In some embodiments, esketamine is administered in a regimen with one to five antidepressants. In other embodiments, esketamine is administered in a regimen with one, two, three, four, or five antidepressants. In other embodiments, esketamine is administered in a regimen with one or two antidepressants. In further embodiments, the esketamine is administered in a regimen with the antidepressant currently being administered to the patient. In other embodiments, the esketamine is administered in a regimen with a different antidepressant. In yet further embodiments, the esketamine is administered in a regimen with an antidepressant not previously administered to the patient. In still other embodiments, the esketamine is administered in a regimen with an antidepressant previously administered to the patient. Where the esketamine and the antidepressant(s) are administered in separate dosage forms, the number of dosages administered per day for each compound may be the same or different and more typically different. The antidepressant may be dosed as prescribed by the attending physician and/or by its label and the esketamine is dosed as described herein. Typically, a patient is under concurrent treatment with both an antidepressant and esketamine, where both are administered by their prescribed dosing regimens.

The esketamine and the antidepressant(s) may be administered via the same or different routes of administration. Examples of suitable methods of administration include, but are not limited to, oral, intravenous (iv), intranasal (in) intramuscular (im), subcutaneous (sc), transdermal, buccal, or rectal. In some embodiments, esketamine is administered intranasally.

As used herein, unless otherwise noted, the term “antidepressant” shall mean any pharmaceutical agent which can be used to treat depression. Suitable examples include, without limitation, a mono-amine oxidase inhibitor, tricyclic, serotonin reuptake inhibitor, serotonin noradrenergic reuptake inhibitor, noradrenergic and specific serotonergic agent, or atypical antipsychotic. Other examples include, but are not limited to mono-amine oxidase inhibitors such as phenelzine, tranylcypromine, moclobemide, and the like: tricyclics such as imipramine, amitriptyline, desipramine, nortriptyline, doxepin, protriptyline, trimipramine, clomipramine, amoxapine, and the like: tetracyclics such as maprotiline, and the like: non-cyclics such as nomifensine, and the like: triazolopyridines such as trazodone, and the like: serotonin reuptake inhibitors such as fluoxetine, sertraline, paroxetine, citalopram, citalopram, escitalopram, fluvoxamine, and the like: serotonin receptor antagonists such as nefazadone, and the like: serotonin noradrenergic reuptake inhibitors such as venlafaxine, milnacipran, desvenlafaxine, duloxetine, levomilnacipran and the like: noradrenergic and specific serotonergic agents such as mirtazapine, and the like: noradrenaline reuptake inhibitors such as reboxetine, edivoxetine and the like: atypical antipsychotics such as bupropion, and the like: natural products such as Kava-Kava, St. John's Wort, and the like: dietary supplements such as s-adenosylmethionine, and the like; and neuropeptides such as thyrotropin-releasing hormone and the like: compounds targeting neuropeptide receptors such as neurokinin receptor antagonists and the like; and hormones such as triiodothyronine, and the like. In some embodiments, the antidepressant is imipramine, amitriptyline, desipramine, nortriptyline, doxepin, protriptyline, trimipramine, maprotiline, amoxapine, trazodone, bupropion, clomipramine, fluoxetine, duloxetine, escitalopram, citalopram, sertraline, paroxetine, fluvoxamine, nefazadone, venlafaxine, milnacipran, reboxetine, mirtazapine, phenelzine, tranylcypromine, moclobemide, Kava-Kava, St. John's Wart, s-adenosylmethionine, thyrotropin releasing hormone, a neurokinin receptor antagonist, or triiodothyronine. Preferably, the antidepressant is selected from the group consisting of fluoxetine, imipramine, bupropion, venlafaxine and sertraline.

Therapeutically effective amounts/dosage levels and dosage regimens for antidepressants (for example, mono-amine oxidase inhibitors, tricyclics, serotonin reuptake inhibitors, serotonin noradrenergic reuptake inhibitors, noradrenergic and specific serotonergic agents, noradrenaline reuptake inhibitor, natural products, dietary supplements, neuropeptides, compounds targeting neuropeptide receptors, hormones and other pharmaceutical agents disclosed herein), may be readily determined by one of ordinary skill in the art. For example, therapeutic dosage amounts and regimens for pharmaceutical agents approved for sale are publicly available, for example as listed on packaging labels, in standard dosage guidelines, in standard dosage references such as the Physician's Desk Reference (Medical Economics Company or online at http://www.pdrel.com) or other sources.

As used herein the term “antipsychotic” includes, but is not limited to:
(a) typical or traditional antipsychotics, such as phenothiazines (e.g., chlorpromazine, thioridazine, fluphenazine, perphenazine, trifluoperazine, levomepromazin), thioxanthenes (e.g., thiothixene, flupentixol), butyrophenones (e.g., haloperidol), dibenzoxazepines (e.g., loxapine), dihydroindolones (e.g., molindone), substituted benzamides (e.g., sulpride, amisulpride), and the like; and
(b) atypical antipsychotics and mood stabilizers, such as paliperidone, clozapine, risperidone, olanzapine, quetiapine, zotepine, ziprasidone, iloperidone, perospirone, blonanserin, sertindole, ORG-5222 (Organon), and the like; and others such as sonepiprazole, aripiprazole, nemonapride, SR-31742 (Sanofi), CX-516 (Cortex), SC-111 (Scotia), NE-100 (Taisho), divalproate (mood stabilizer) and the like.

In an embodiment, the “atypical antipsychotic” is selected from the group consisting of aripiprazole, quetiapine, olanzapine, risperidone and paliperidone. In another embodiment, the atypical antipsychotic is selected from the group consisting of aripiprazole, quetiapine, olanzapine and risperidone: preferably, the atypical antipsychotic is selected from the group consisting of aripiprazole, quetiapine and olanzapine.

One skilled in the art will recognize that the failure to respond to an adequate course of a given antidepressant may be determined retrospectively or prospectively. In an embodiment, at least one of the failures to respond to an adequate course of antidepressant is determined prospectively. In another embodiment, at least two of the failures to respond to an adequate course of antidepressant are determined prospectively. In another embodiment, at least one of the failures to respond to an adequate course of antidepressant is determined retrospectively. In another embodiment, at least two of the failures to respond to an adequate course of antidepressant are determined retrospectively in a current depressive episode.

The “at least two oral antidepressants” or “at least two different oral depressants” has been administered to the patient at an adequate dose which may be determined by the attending physician. Similarly, the antidepressant has been administered for a suitable duration, as determined by the attending physician.

As used herein, unless otherwise noted, the terms “treating”, “treatment” and the like, shall include the management and care of a subject or patient (preferably mammal, more preferably human) for the purpose of combating a disease, condition, or disorder and includes the administration of a compound described herein to prevent the onset of the symptoms or complications, alleviate the symptoms or complications, or eliminate the disease, condition, or disorder.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In some embodiments, the antidepressant is utilized in a therapeutically effective amount as determined by the attending physician. In other embodiments, esketamine is utilized in a therapeutically effective amount.

As used herein, the term “composition” is intended to encompass a product comprising the specified ingredients in the specified amounts, as well as any product which results, directly or indirectly, from combinations of the specified ingredients in the specified amounts.

In some embodiments, the induction period may be said to have completed when a patient's MADRS score is reduced by ≥50% from baseline or from about 20 to about

13. In other embodiments, the patient's MADRS score may be about 19, about 18, about 17, about 16, about 15, about 14, or about 13. Patients with MADRS scores≤12 are considered in remission and if stable for four weeks should be moved to or maintained in the maintenance phase.

### Pharmaceutical Compositions

The preferred pharmaceutical composition contains esketamine hydrochloride as the active ingredient is intimately admixed with a pharmaceutical carrier, preferably water, according to conventional pharmaceutical compounding techniques, which carrier may take a wide variety of forms depending of the form of preparation desired for administration. Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al: Pharmaceutical Dosage Forms: Parenteral Medications, Volumes 1-2, edited by Avis et al; and Pharmaceutical Dosage Forms: Disperse Systems, Volumes 1-2, edited by Lieberman et al: published by Marcel Dekker, Inc.

One suitable aqueous formulation of esketamine, comprises water and esketamine: wherein the esketamine is present in an amount in the range of from about 25 mg/mL to about 250 mg/mL, preferably about 55 mg/mL to about 250 mg/mL or about 100 mg/mL to about 250 mg/mL, or any amount or range therein, based on the total volume of the pharmaceutical composition. Preferably, the esketamine is present in an amount in the range of from about 150 mg/ml to about 200 mg/mL, or any amount or range therein. More preferably, the esketamine is present in an amount in the range of from about 150 mg/mL to about 175 mg/mL, or any amount or range therein. More preferably, the esketamine is present in an amount in the range of from about 160 mg/mL to about 163 mg/mL, for example, in an amount of about 161.4 mg/mL

Another suitable aqueous formulation of esketamine comprises water and esketamine: wherein the esketamine is present in an amount in the range of from about eq. 100 mg/mL to about eq. 250 mg/mL, or any amount or range therein, based on the total volume of the pharmaceutical composition. Preferably, the esketamine is present in an amount in the range of from about eq. 125 mg/ml to about eq. 180 mg/mL, or any amount or range therein. More preferably, the esketamine is present in an amount in the range of from about eq. 140 mg/mL to about eq. 160 mg/mL, or any amount or range therein, for example, in an amount of about eq. 140 mg/mL.

Suitable pharmaceutical compositions for use herein are preferably an aqueous formulation. As used herein, unless otherwise noted, the term “aqueous” shall mean that the primary liquid component of the formulation is water. Preferably, water constitutes greater than about 80 wt-% of the liquid component of the pharmaceutical composition, more preferably greater than about 90 wt-%, more preferably greater than about 95 wt-%, more preferably about 98 wt-%.

In suitable pharmaceutical compositions for use herein, the water content of the composition is within the range of 85±14 wt.-%, more preferably 85±12 wt.-%, still more preferably 85±10 wt.-%, most preferably 85±7.5 wt.-% and in particular 85±5 wt.-%, based on the total weight of the composition.

In suitable pharmaceutical compositions for use herein, preferably the water content of the composition is within the range of 90±14 wt.-%, more preferably 90±12 wt.-%, still more preferably 90±10 wt.-%, most preferably 80±7.5 wt.-% and in particular 90±5 wt.-%, based on the total weight of the composition.

In another pharmaceutical composition for use herein, the water content of the composition is within the range of 95±4.75 wt.-%, more preferably 95±4.5 wt.-%, still more preferably 95±4 wt.-%, yet more preferably 95±3.5 wt.-%, most preferably 95±3 wt.-% and in particular 95±2.5 wt.-%, based on the total weight of the composition.

In another pharmaceutical composition for use herein, the water content of the composition is within the range of from 75 to 99.99 wt.-%, more preferably 80 to 99.98 wt.-%, still more preferably 85 to 99.95 wt.-%, yet more preferably 90 to 99.9 wt.-%, most preferably 95 to 99.7 wt.-% and in particular 96.5 to 99.5 wt.-%, based on the total weight of the composition.

In another pharmaceutical composition for use herein, the composition further comprises one or more buffers and/or buffer systems (i.e. conjugate acid-base-pairs).

As used herein, the term “buffer” shall mean any solid or liquid composition (preferably an aqueous, liquid composition) which when added to an aqueous formulation adjusts the pH of said formulation. One skilled in the art will recognize that a buffer may adjust the pH of the aqueous formulation in any direction (toward more acidic, more basic or more neutral pH). Preferably, the buffer is pharmaceutically acceptable.

Suitable examples of buffers which may be used in the aqueous formulations described herein include, but are not limited to citric acid, sodium dihydrogen phosphate, disodium hydrogen phosphate, acetic acid, boric acid, sodium borate, succinic acid, tartaric acid, malic acid, lactic acid, furmaric acid, and the like. Preferably, the buffer or buffer system is selected from the group consisting of NaOH, citric acid, sodium dihydrogen phosphate and disodium hydrogen phosphate.

In an embodiment, the buffer is selected to adjust the pH of the esketamine hydrochloride pharmaceutical compositions herein (e.g. the aqueous formulations described herein) into a pH in the range of from about pH 3.5 to about pH 6.5, or any amount or range therein. Preferably, the buffer is selected to adjust the pH of the esketamine hydrochloride compositions herein to about in the range of from about pH 4.0 to about pH 5.5, or any amount or range therein, more preferably, in the range of from about pH 4.5 to about pH 5.0, or any amount or range therein.

Preferably, the concentration of the buffer and buffer system, respectively, preferably NaOH, is adjusted to provide a sufficient buffer capacity.

In an embodiment, the pharmaceutical composition comprises esketamine hydrochloride, water, and a buffer or buffer system, preferably NaOH: wherein the buffer or buffer system is present in an amount sufficient to yield a formulation with a pH in the range of from about pH 4.0 to about pH 6.0, or any amount or range therein.

Optionally the pharmaceutical compositions herein may contain a preservative.

As used herein, unless otherwise noted, the terms “antimicrobial preservative” and “preservative” preferably refer to any substance that is usually added to pharmaceutical compositions in order to preserve them against microbial degradation or microbial growth. In this regard, microbial growth typically plays an essential role, i.e. the preservative serves the main purpose of avoiding microbial contamination. As a side aspect, it may also be desirable to avoid any effect of the microbes on the active ingredients and excipients, respectively, i.e. to avoid microbial degradation.

Representative examples of preservatives include, but are not limited to, benzalkonium chloride, benzethonium chloride, benzoic acid, sodium benzoate, benzyl alcohol, bronopol, cetrimide, cetylpyridinium chloride, chlorhexidine, chlorbutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, sodium propionate, thimerosal, methyl paraben, ethyl paraben, propyl paraben, butyl paraben, isobutyl paraben, benzyl paraben, sorbic acid, and potassium sorbate.

The complete absence of preservatives in the pharmaceutical compositions used herein is preferred when the content of esketamine hydrochloride is sufficiently high so that due to its preservative property the desired shelf life or in use stability can be achieved by the presence of the drug itself. Preferably, under these circumstances the concentration of esketamine hydrochloride is at least eq. 120 mg/mL, preferably in the range of from about eq. 120 mg/mL to about eq. 175 mg/ml, or any amount or range therein, more preferably in an amount in the range of from about eq. 125 mg/mL to about eq. 150 mg/mL, or any amount or range therein, for example at about eq. 126 mg/mL or at about eq. 140 mg/mL.

As used herein, the terms “penetration agent”, “penetration enhancer”, and “penetrant” refer to any substance that increases or facilitates absorption and/or bioavailability of the active ingredient (e.g. esketamine hydrochloride) of a pharmaceutical composition. Preferably, the penetration agents increases or facilitates absorption and/or bioavailability of the active ingredient (e.g. esketamine hydrochloride) of a pharmaceutical composition, following nasal administration (i.e. increases or facilitates absorption and/or bioavailability of the active ingredient through the mucosal membrane).

Suitable examples include, but are not limited to tetradecyl maltoside, sodium glycocholate, tauroursodeoxycholic acid (TUDCA), lecithines, and the like; and chitosan (and salts), and surface active ingredients such as benzalkonium chloride, sodium dodecyl sulfate, sodium docusate, polysorbates, laureth-9, oxtoxynol, sodium deoxycholate, polyarginine, and the like. Preferably, the penetration agent is tauroursodeoxycholic acid (TUDCA).

The penetration agent may work via any mechanism, including for example by increasing the membrane fluidity, creating transient hydrophilic pores in the epithelial cells, decreasing the viscosity of the mucus layer or opening up tight junctions. Some penetration agents (for example bile salts and fusidic acid derivatives) may also inhibit the enzymatic activity in the membrane, thereby improving bioavailability of the active ingredient.

Preferably, the penetration agent is selected to meet one or more, more preferably all, of the following general requirements:
(a) It is effective at increasing absorption (preferably nasal absorption) of the active ingredient, preferably in a temporary and/or reversible manner;
(b) It is pharmacologically inert;
(c) It is non-allergic, non-toxic and/or non-irritating;
(d) It is highly potent (effective in small amounts);
(e) It is compatible with the other components of the pharmaceutical composition;
(f) It is odorless, colorless and/or tasteless;
(g) It is accepted by regulatory agencies; and
(h) It is inexpensive and available in high purity.

In one embodiment, the penetration agent is selected to increase penetration (absorption and/or bioavailability of the esketamine hydrochloride) without nasal irritation. In another embodiment, the penetration agent is selected to improve absorption and/or bioavailability of the esketamine hydrochloride; and further selected to enhance uniform dosing efficacy.

In an embodiment, the pharmaceutical composition comprises esketamine and water: herein the pharmaceutical composition does not contain an antimicrobial preservative; and wherein the pharmaceutical compositions further contains a penetration enhancer, preferably TUDCA.

In another embodiment, the pharmaceutical composition comprises esketamine and water: herein the pharmaceutical composition does not contain an antimicrobial preservative; and wherein the pharmaceutical compositions further contains tauroursodeoxycholic acid (TUDCA): wherein the TUDCA is present in a concentration in the range of from about 1.0 mg/mL to about 25.0 mg/mL, or any amount or range therein, preferably in a concentration in the range of from about 2.5 mg/mL to about 15 mg/mL, or any amount or range therein, preferably in a concentration in the range of from about 5 mg/mL to about 10 mg/mL, or any amount or range therein. In another embodiment, the TUDCA is present at a concentration of about 5 mg/mL. In a further embodiment, the TUDCA is present at a concentration of about 10 mg/mL.

The pharmaceutical compositions for use herein may further contain one or more additional excipients for example, wetting agents, surfactant components, solubilizing agents, thickening agents, colorant agents, antioxidant components, and the like.

Examples of a suitable antioxidant component, if used, include, but are not limited to one or more of the following: sulfites: ascorbic acid: ascorbates, such as sodium ascorbate, calcium ascorbate, or potassium ascorbate: ascorbyl palmitate: fumaric acid: ethylene diamine tetraacetic acid (EDTA) or its sodium or calcium salts: tocopherol: gallates, such as propyl gallate, octyl gallate, or dodecyl gallate: vitamin E; and mixtures thereof. The antioxidant component provides long term stability to the liquid compositions. Addition of the antioxidant component can help enhance and ensure the stability of the compositions and renders the compositions stable even after six months at 40° C. A suitable amount of the antioxidant component, if present, is about 0.01 wt.-% to about 3 wt.-%, preferably about 0.05 wt.-% to about 2 wt.-%, of the total weight of the composition.

Solubilizing and emulsifying agents can be included to facilitate more uniform dispersion of the active ingredient or other excipient that is not generally soluble in the liquid carrier. Examples of a suitable emulsifying agent, if used, include, but are not limited to, for example, gelatin, cholesterol, acacia, tragacanth, pectin, methyl cellulose, carbomer, and mixtures thereof. Examples of a suitable solubilizing agent include polyethylene glycol, glycerin, D-mannitol, trehalose, benzyl benzoate, ethanol, trisaminomethane, cholesterol, triethanolamine, sodium carbonate, sodium citrate, sodium salicylate, sodium acetate, and mixtures thereof.

Preferably, the solubilizing agent includes glycerin. The solubilizing or emulsifying agent is/are generally present in an amount sufficient to dissolve or disperse the active ingredient, i.e. esketamine, in the carrier. Typical amounts when a solubilizing or an emulsifier are included are from about 1 wt.-% to about 80 wt.-%, preferably about 20 wt.-% to about 65 wt.-%, and more preferably about 25 wt.-% to about 55 wt.-%, of the total weight of the composition.

A suitable isotonizing agent, if used, includes sodium chloride, glycerin, D-mannitol, D-sorbitol, glucose, and mixtures thereof. A suitable amount of the isotonizing agent, when included, is typically about 0.01 wt.-% to about 15 wt.-%, more preferably about 0.3 wt.-% to about 4 wt.-%, and more preferably about 0.5 wt.-% to about 3 wt.-%, of the total weight of the composition.

A suspending agent or viscosity increasing agent can be added to the pharmaceutical compositions, to for example, increase the residence time in the nose. Suitably examples include, but are not limited to, hydroxypropyl methylcellulose, sodium carmellose, microcrystalline cellulose, carbomer, pectin, sodium alginate, chitosan salts, gellan gum, poloxamer, polyvinyl pyrrolidone, xanthan gum, and the like.

Advantageously, esketamine may be administered in a single daily dose, or the total daily dosage may be administered in divided doses of two, three or four times daily, preferably two times daily. Typically, divided doses should be made closer in time. In some embodiments, divided doses are administered about within 20 minutes, about 15 minutes, about 10 minutes, about 5 minutes, about 4 minutes, about 3 minutes, about 2 minutes, about 1 minute, or less of each other. Additionally, in a flexible dosing regimen a patient could be dosed daily, twice a week, once a week, once every other week or once monthly. For example, one dose of the esketamine is administered on day 1 and another dose of the esketamine is administered on day 2, or one dose of the esketamine is administered on day 1 and another dose of the esketamine is administered on day 3, or one dose of the esketamine is administered on day 1 and another dose of the esketamine is administered on day 4, or one dose of the esketamine is administered on day 1 and another dose of the esketamine is administered on day 5. Furthermore, esketamine is preferably administered in intranasal form via topical use of suitable intranasal vehicles, such as a nasal spray pump.

### Nasal Device

A representative nasal spray device is disclosed in U.S. Pat. No. 6,321,942, and U.S. Patent Application Publication No. 2020-0009081A1, both incorporated by reference herein. For example, a disposable atomizer for discharging successive partial discharge amounts as a spray may be utilized to carry out the methods discloses herein. Typically, such devices allow a medicament to be sprayed into both nostrils of a patient in two successive strokes. The device may be ready-to-use wherein the medicament is discharged from a medium container. The device is typically able to separate a first discharge stroke from a second discharge stroke to prevent complete emptying of the medium container in a single motion. The device may take the form of a double-stroke disposable pump, which is disposed of after a single use and enables individual partial discharges with high dosing precision and reliability.

In one embodiment, the nasal spray device is a single-use device that delivers a total of 28 mg of esketamine in two sprays, one spray per nostril. The device may be operated by the patient under the supervision of a healthcare professional. With respect to dosage amounts, one device may be used for a 28 mg dose, two devices for a 56 mg dose, or three devices for an 84 mg dose. It is also preferable to have a 5-minute interval between the use of each device. As described in Example 2, time 0 is defined as the time of administration of the first intranasal spray to one nostril from the first intranasal device.

### Aspects

Aspect 1. A method of treating depression in a human patient in need thereof, said method having an induction phase and treatment sessions, wherein the induction phase has a duration of 4 weeks, the method comprising:
intranasally administering about 56 mg or about 84 mg of esketamine per induction phase treatment session to said patient, wherein the induction phase treatment session occurs at a frequency of twice weekly during the induction phase: wherein prior to any treatment session, the patient has a systolic blood pressure of less than 140 mmHg and a diastolic blood pressure of less than 110 mmHg; and
after at least the first two treatment sessions, monitoring the patient following each treatment session for a period of at least 90 minutes for adverse events; and, if the patient does not experience a severe adverse event and does not experience any of the following adverse events:
clinically meaningful blood pressure increases,
moderate or greater levels of dissociation,
moderate or greater levels of sedation,
moderate or greater levels of dizziness, and
moderate of greater levels of vertigo, following any two consecutive treatment sessions;
the patient becomes a qualified patient for a post-treatment session monitoring period of less than 90 minutes in a next treatment session.

Aspect 2. The method of Aspect 1, wherein the patient becomes a qualified patient after at least the first three, four, five, six, seven, or eight treatment sessions.

Aspect 3. The method of any one of the previous Aspects, wherein the qualified patient post-treatment session monitoring period is at least 60 minutes.

Aspect 4. The method of any one of the previous Aspects, wherein a treatment session is rescheduled if, prior to the treatment session, the patient does not have a systolic blood pressure of less than 140 mmHg and a diastolic blood pressure of less than 110 mmHg.

Aspect 5. The method of any one of the previous claims, wherein the patient does not have current uncontrolled hypertension.

Aspect 6. The method of any one of the previous Aspects, wherein the patient is not taking medication or substances that promote sedation or blood pressure increases.

Aspect 7. The method of any one of the previous Aspects, wherein the patient is less than 65 years of age.

Aspect 8. The method of any one of the previous Aspects, wherein the depression is major depressive disorder.

Aspect 9. The method of Aspect 8, wherein the depression is major depressive disorder with suicidal ideation or severe major depressive disorder.

10. The method of any one of Aspects 1-9, wherein the method further comprises a subsequent maintenance phase comprising intranasally administering about 56 mg or about 84 mg of esketamine per maintenance phase treatment session to said patient, wherein the maintenance phase treatment session occurs at a frequency of once weekly for the first four weeks of the maintenance phase and is adjusted to once weekly or once every other week thereafter.

Aspect 11. The method of Aspect 10, wherein the depression is major depressive disorder.

Aspect 12. The method of Aspect 11, wherein the depression is treatment resistant depression.

Aspect 13. The method of any one of the previous Aspects, comprising adjunctive treatment with a therapeutically effective amount of one or more antidepressants during the induction phase and the maintenance phase.

Aspect 14. The method of Aspect 13, wherein the one or more antidepressants are administered at least 3 hours after any induction phase or maintenance phase treatment session.

Aspect 15. The method of Aspect 1, wherein in the next treatment session the qualified patient is intranasally administering about 56 mg or about 84 mg of esketamine and monitored in the post-treatment session monitoring period for less than 90 minutes and released after a healthcare professional determines that the qualified patient is clinically stable and discharge ready.

The following Examples are set forth to aid in the understanding of the invention, and are not intended and should not be construed to limit in any way the invention set forth in the claims which follow thereafter.

### EXAMPLES

### Example 1

Esketamine was supplied as a clear, colorless intranasal solution of esketamine hydrochloride (16.14% weight/volume [w/v]: equivalent to 14% w/v of esketamine base) in a nasal spray pump. The solution consists of 161.4 mg/mL esketamine hydrochloride (equivalent to 140 mg of esketamine base) formulated in 0.12 mg/mL ethylenediaminetetraacetic acid (EDTA) and 1.5 mg/mL citric acid at a pH of 4.5 in water for injection. It is provided in a nasal spray pump, which delivers 16.14 mg esketamine hydrochloride (14 mg esketamine base) per 100-μL spray. Each individual nasal spray pump (device) contains a total of 28 mg (i.e., 2 sprays).

### Example 2

Data were pooled from adult patients (aged 18-64 years) with TRD. Patients had recurrent or single-episode (>2 years) MDD (DSM-5), a Clinician-Rated Inventory of Depressive Symptomatology score of >3 and a total Montgomery-Åsberg Depression Rating Scale (MADRS) score of >2. At screening, depressive symptoms had been non-responsive to adequate trials of ≥1 and ≤5 commercially available OAD in the current depressive episode. Non response was further confirmed by a prospective trial of a different OAD lasting ≥4 weeks and during the screening phase.

### A. Study Designs

In brief, study 1 was a double-blind, placebo-controlled relapse prevention trial comparing the efficacy of ESK versus placebo nasal spray, both in conjunction with a Food and Drug Administration (FDA)-approved OAD, in delaying relapse of depressive symptoms in patients with TRD who were either in stable remission or stable response after four months' treatment with ESK in conjunction with a FDA-approved OAD. Study 2 was an open-label, multicenter study that assessed the long-term safety and efficacy of ESK in conjunction with an FDA-approved OAD in patients with TRD.

The analysis was limited to the two doses of ESK (56 mg and 84 mg) approved for use by the FDA for patients with TRD. Patients received ESK (56 or 84 mg) in conjunction with an OAD twice weekly during the induction phase (Weeks 1-4); once weekly during Weeks 5-8 of the optimization phase: either once weekly or every other week during the remainder of the 12-week optimization phase and the maintenance phase (individualized according to the severity of depressive symptoms [MADRS total score≤/>12] and tolerability).

### B. Safety Assessments

Patients were monitored for >90 minutes after ESK administration at each treatment session, after which time they could leave the clinical site at the clinician's discretion. AEs were monitored and reported, and safety assessments, including clinical laboratory tests and physical examinations, were conducted throughout the study. Clinician-reported AEs were categorized as mild, moderate, or severe based on clinical judgement (Table 1).

TABLE 1
Classification of Clinician-Reported Adverse Events

| Severity | Description |
|---|---|
| Mild | Awareness of symptoms that are easily tolerated, |
| | causing minimal discomfort and not |
| | interfering with everyday activities |
| Moderate | Sufficient discomfort present to cause some |
| | interference with normal activity |
| Severe | Extreme distress, causing significant impairment |
| | of functioning or incapacitation and |
| | preventing normal everyday activities |

Vital signs, the Clinician-Administered Dissociative States Scale (CADSS), and the Modified Observer's Assessment of Alertness/Sedation (MOAA/S) were assessed at baseline and all treatment sessions (predose, and at 40 minutes, 1 hour [vital signs only unless spontaneously reported by the patient], and 1.5 hours postdose). Routine blood pressure (BP) readings were conducted at each treatment session, with elevations reported as AEs based on the clinician's judgement. The CADSS assessed treatment-emergent dissociative symptoms and consists of 23 questions, each coded on a 5-point scale (0=not at all, 1-mild, 2-moderate, 3=severe, 4-extreme), yielding a total score 0-92.11 A CADSS total score >4 indicated the presence of dissociative symptoms. The MOAA/S assessed treatment-emergent sedation: scores range from (=no response to painful stimulus to 5=readily responds to name spoken in normal tone. Any decrease in MOAA/s from pre-dose (score <5) indicated some degree of sedation. An abnormal elevation in BP was defined as systolic BP ≥180 mmHg and ≥20 mmHg higher than baseline and/or diastolic BP ≥105 mmHg and ≥15 mmHg greater than baseline.

### C. Analysis of the Relationship Between Early AE and AE Recurrence

This analysis included any treatment sessions in which patients received ESK (either open-label or blinded). The occurrence and severity of AEs were assessed within the following timeframes: Month 1 (Week 1 and Weeks 2-4), Month 2 (Weeks 5-8), Months 3-6 and Months 6-12. Severity of clinician-reported AEs were scored as 0 (no AE), 1 (mild), 2 (moderate), and 3 (severe). To examine AEs in individual patients over time, only patients who received at least one ESK dose in the following period were included in the analyses of each respective period. Thus, patient data were retained in (i) Week 1 if the patient received ≥1 ESK dose in Weeks 2-4, (ii) Weeks 2-4 if the patient received ≥1 ESK dose in Weeks 5-8, (iii) Weeks 5-8 if the patient received ≥1 ESK dose in Months 3-6, and (iv) Months 3-6 if the patient received ≥1 ESK dose in Months 6-12.

The five most commonly reported treatment-emergent events occurring with ESK plus OAD therapy (dissociation, dizziness, nausea, sedation, and vertigo) were evaluated, each of which has an incidence ≥5% and at least a 2-fold greater incidence than with placebo plus an OAD. 14 Increases in BP were also examined.

Week 1 and Week 4 were set a priori as index weeks because they are the beginning and end of the induction period. Frequencies and maximum-reported severities of AEs reported during these index weeks served as stratifying variables by which to examine recurrence and severities of AE recurrences in future timeframes.

Patients who received medication either to treat an emergent AE or prophylactically to prevent the (re) emergence of an AE, were identified and the potential role in influencing rates of AE recurrence examined.

Data were summarized with descriptive statistics.

### D. Results

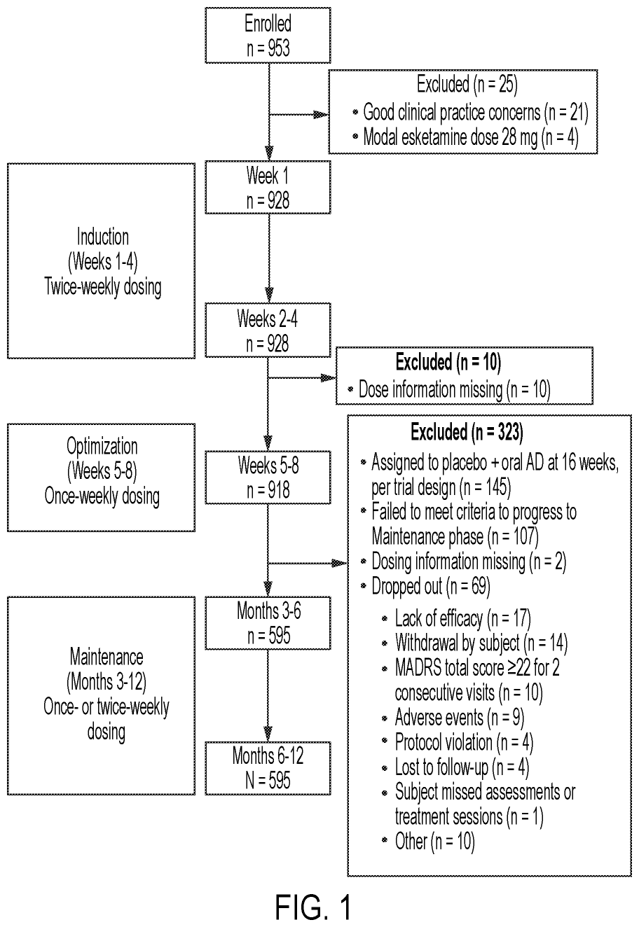

Of 953 patients, 25 were excluded from all analyses, 21/25 because of concerns regarding study site conduct (FIG. 1); sensitivity analyses including these excluded patients confirmed no impact on the overall conclusions of the study. The dataset thus included 928 patients in Weeks 1-4, 918 patients in Weeks 5-8, and 595 patients in Months 3-6 and 6-12. Discontinuations, including those due to AEs, have been previously reported and the reason for patients being excluded in subsequent timeframes within this analysis was primarily protocol-defined randomization to placebo in study 1. Mean age of the patients was 46 years, and approximately two thirds of patients were female (Table 2).

TABLE 2
Patient demographics and baseline characteristics

| | Patients | Patients |
|---|---|---|
| | included in | included in |
| | Weeks 1-4 | Month 3-12 |
| | n = 928 | n = 595 |
| Mean age, years (SD) | 46.3 (11.1) | 46.4 (11.2) |
| Males, n (%) | 328 (35.3) | 215 (36.1) |
| Race, n (%) | | |
| White | 807 (87.0) | 509 (85.5) |
| Black or African American | 31 (3.3) | 18 (3.0) |
| Asian | 53 (5.7) | 47 (7.9) |
| Other/unknown | 36 (3.9) | 21 (2.0) |
| Ethnicity | | |
| Hispanic | 175 (18.9) | 125 (21.0) |
| Non-Hispanic | 740 (79.7) | 462 (77.6) |
| Other/unknown | 13 (1.4) | 8 (1.3) |
| Mean body mass index, kg/m², (SD) | 28.3 (6.0) | 28.2 (6.0) |
| History of hypertension, n (%) | 194 (20.9) | 124 (20.8) |

(i) Association of Frequency of AE Incidence in Week 1 with Later Recurrence

Across the examined AEs, the more frequently an AE occurred during the first week of treatment, the higher the percentage of patients suffering a recurrence (Table 3).

TABLE 3
Rates of adverse event recurrence with esketamine nasal spray according to frequency of adverse events in Week 1

| | Clinician-reported dissociation, % (n/N) | | | | CADSS-defined dissociation, % (n/N) | | | | Dizziness, % (n/N) | | | |
|---|---|---|---|---|---|---|---|---|---|---|---|---|
| | Overall | No | AE | AE | Overall | No | AE | AE | Overall | No | AE | AE |
| | rate for | AE in | once in | twice in | rate for | AE in | once in | twice in | rate for | AE in | once in | twice in |
| | timeframe | Week 1 | Week 1 | Week 1 | timeframe | Week 1 | Week 1 | Week 1 | timeframe | Week 1 | Week 1 | Week 1 |
| Week | 17.1 | | — | | 56.6 | | — | | 21.1 | | — | |
| 1 | (159/928) | | | | (525/928) | | | | (196/928) | | | |
| Weeks | 16.1 | 5.7 | 48.2 | 86.5 | 53.1 | 24.8 | 59.1 | 88.8 | 19.2 | 6.3 | 51.9 | 86.4 |
| 2-4 | (149/928) | (44/769) | (41/85) | (64/74) | (493/928) | (100/403) | (146/247) | (247/278) | (178/928) | (46/732) | (56/108) | (76/88) |
| Weeks | 12.6 | 6.1 | 29.8 | 60.0 | 34.9 | 15.6 | 32.5 | 64.6 | 12.5 | 3.7 | 30.8 | 62.5 |
| 5-8 | (116/918) | (46/759) | (25/84) | (45/75) | (320/918) | (62/398) | (79/243) | (179/277) | (115/918) | (27/723) | (33/107) | (55/88) |
| Months | 14.4 | 7.6 | 26.2 | 61.1 | 38.6 | 19.4 | 42.7 | 61.3 | 15.6 | 7.5 | 31.2 | 53.0 |
| 3-6 | (86/595) | (36/476) | (17/65) | (33/54) | (230/595) | (49/252) | (67/157) | (114/186) | (93/595) | (34/452) | (24/77) | (35/66) |
| Months | 8.8 | 4.6 | 18.5 | 33.3 | 25.9 | 15.9 | 26.8 | 38.7 | 8.1 | 4.0 | 20.8 | 21.2 |
| 6-12 | (52/595) | (22/476) | (12/65) | (18/54) | (154/595) | (40/252) | (42/157) | (72/186) | (48/595) | (18/452) | (16/77) | (14/66) |
| | Clinician-reported sedation, % (n/N) | | | | MOAA/S-defined sedation, % (n/N) | | | | Nausea, % (n/N) | | | |
| | Overall | No | AE | AE | Overall | No | AE | AE | Overall | No | AE | AE |
| | rate for | AE in | once in | twice in | rate for | AE in | once in | twice in | rate for | AE in | once in | twice in |
| | timeframe | Week 1 | Week 1 | Week 1 | timeframe | Week 1 | Week 1 | Week 1 | timeframe | Week 1 | Week 1 | Week 1 |
| Week | 3.9 | | — | | 38.1 | | — | | 14.3 | | — | |
| 1 | (36/928) | | | | (354/928) | | | | (133/928) | | | |
| Weeks | 4.1 | 2.1 | 36.8 | 70.6 | 44.2 | 22.0 | 65.7 | 95.4 | 9.4 | 5.3 | 28.6 | 61.9 |
| 2-4 | (38/928) | (19/892) | (7/19) | (12/17) | (410/928) | (126/574) | (119/181) | (165/173) | (87/928) | (42/795) | (32/112) | (13/21) |
| Weeks | 2.6 | 1.2 | 22.2 | 52.9 | 33.1 | 15.8 | 42.0 | 81.4 | 4.0 | 1.7 | 16.1 | 30.0 |
| 5-8 | (24/918) | (11/883) | (4/18) | (9/17) | (304/918) | (90/570) | (74/176) | (140/172) | (37/918) | (13/786) | (18/112) | (6/20) |
| Months | 3.5 | 3.0 | 0 | 36.4 | 37.5 | 23.8 | 47.7 | 74.5 | 7.2 | 4.5 | 20.8 | 33.3 |
| 3-6 | (21/595) | (17/575) | (0/9) | (4/11) | (223/595) | (90/378) | (51/107) | (82/110) | (43/595) | (23/506) | (16/77) | (4/12) |
| Months | 2.5 | 1.7 | 0 | 45.5 | 28.4 | 18.8 | 38.3 | 51.8 | 6.7 | 4.9 | 18.2 | 8.3 |
| 6-12 | (15/595) | (10/575) | (0/9) | (5/11) | (169/595) | (71/378) | (41/107) | (57/110) | (40/595) | (25/506) | (14/77) | (1/12) |

| | Clinician-reported increased | | | | Measured-based increased | | | | | | | |
|---|---|---|---|---|---|---|---|---|---|---|---|---|
| | blood pressure, % (n/N) | | | | blood pressure, % (n/N) | | | | Vertigo, % (n/N) | | | |
| | Overall | No | AE | AE | Overall | No | AE | AE | Overall | No | AE | AE |
| | rate for | AE in | once in | twice in | rate for | AE in | once in | twice in | rate for | AE in | once in | twice in |
| | timeframe | Week 1 | Week 1 | Week 1 | timeframe | Week 1 | Week 1 | Week 1 | timeframe | Week 1 | Week 1 | Week 1 |
| Week | 4.4 | | — | | 3.6 | | — | | 12.4 | | — | |
| 1 | (41/928) | | | | (33/928) | | | | (115/928) | | | |
| Weeks | 5.2 | 2.4 | 56.5 | 77.8 | 4.7 | 3.4 | 35.7 | 80.0 | 15.0 | 5.5 | 67.4 | 90.3 |
| 2-4 | (47/928) | (21/887) | (13/23) | (14/18) | (44/928) | (30/895) | (10/28) | (4/5) | (139/928) | (45/813) | (29/43) | (65/72) |
| Weeks | 3.9 | 1.9 | 40.9 | 55.6 | 3.4 | 2.6 | 17.9 | 60.0 | 9.9 | 3.0 | 45.2 | 67.6 |
| 5-8 | (36/918) | (17/878) | (9/22) | (10/18) | (31/918) | (23/885) | (5/28) | (3/5) | (91/918) | (24/805) | (19/42) | (48/71) |
| Months | 4.2 | 3.0 | 36.4 | 40.0 | 2.7 | 2.4 | 6.3 | 33.3 | 8.2 | 3.4 | 28.6 | 59.5 |
| 3-6 | (25/595) | (17/574) | (4/11) | (4/10) | (16/595) | (14/576) | (1/16) | (1/3) | (49/595) | (18/532) | (6/21) | (25/42) |
| Months | 2.2 | 1.7 | 9.1 | 20.0 | 3.2 | 3.1 | 6.3 | 0 | 5.0 | 1.7 | 23.8 | 38.1 |
| 6-12 | (13/595) | (10/574) | (1/11) | (2/10) | (19/595) | (18/576) | (1/16) | (0/3) | (30/595) | (19/532) | (5/21) | (16/42) |

In each cell, n/N represents the number of patients who experienced a recurrence of the given AE/number of patients who contributed data (based on the time-period described in the row title and the occurrence in Week 1 described in the column title).
Shaded cells depict ≥10% difference in AE recurrence rates between occurrence twice vs once per week in Week 1 (dark gray) and once per week vs none in Week 1 (light gray).

TABLE 3A

| | | |
|---|---|---|
| Postdose | Vomiting, % (n/n) | Feeling Drunk, % (n/n) |

| Monitoring | No. of | | None in | Once in | Twice in | | None in | Once in | Twice in |
|---|---|---|---|---|---|---|---|---|---|
| Period | Patients | Overall | Week 1 | Week 1 | Week 1 | Overall | Week 1 | Week 1 | Week 1 |
| Weeks | 949 | 3.3 | 2.4 | 31.0 | 0 | 1.9 | 0.4 | 30.8 | 90.9 |
| 2-4 | | | (22/912) | (9/29) | (0/8) | | (4/925) | (4/13) | (10/11) |
| Weeks | 918 | 1.8 | 1.5 | 21.1 | 0 | 1.6 | 0.1 | 16.7 | 90.9 |
| 5-8 | | | (13/881) | (4/29) | (0/8) | | (1/894) | (4/13) | (10/11) |
| Months | 595 | 2.5 | 1.9 | 21.1 | 0 | 1.5 | 0.2 | 16.7 | 100.0 |
| 3-6 | | | (11/570) | (4/19) | (0/6) | | (1/582) | (1/6) | (7/7) |
| Months | 595 | 2.2 | 1.9 | 10.5 | 0 | 1.5 | 0.2 | 33.3 | 85.7 |
| 6-12 | | | (11/570) | (2/19) | (0/6) | | (1/582) | (2/6) | (6/7) |

| Postdose | | Hypoesthesia, % (n/n) | | | | Anxiety, % (n/n) | | | |
|---|---|---|---|---|---|---|---|---|---|
| Monitoring | No. of | | None in | Once in | Twice in | | None in | Once in | Twice in |
| Period | Patients | Overall | Week 1 | Week 1 | Week 1 | Overall | Week 1 | Week 1 | Week 1 |
| Weeks | 949 | 7.2 | 3.7 | 54.3 | 76.2 | 1.7 | 1.3 | 11.8 | 100 |
| 2-4 | | | (33/893) | (19/35) | (16/21) | | (12/930) | (2/17) | (2/2) |
| Weeks | 918 | 3.4 | 1.6 | 20.7 | 58.3 | 1.0 | 0.8 | 5.9 | 50.0 |
| 5-8 | | | (14/860) | (7/36) | (10/22) | | (7/899) | (1/17) | (1/2) |
| Months | 595 | 5.2 | 2.5 | 20.7 | 73.3 | 1.5 | 1.2 | 10.0 | 50.0 |
| 3-6 | | | (14/551) | (6/29) | (11/15) | | (7/588) | (1/10) | (1/2) |
| Months | 595 | 3.2 | 1.6 | 13.8 | 40.0 | 2.2 | 2.1 | 0 | 50.0 |
| 6-12 | | | (9/551) | (4/29) | (6/15) | | (12/583) | (0/10) | (1/2) |

| | |
|---|---|
| Postdose | Lethargy, % (n/n) |

| Monitoring | No. of | | None in | Once in | Twice in |
|---|---|---|---|---|---|
| Period | Patients | Overall | Week 1 | Week 1 | Week 1 |
| Weeks | 949 | 1.5 | 1.0 | 71.4 | 0 |
| 2-4 | | | (9/941) | (5/7) | (0/1) |
| Weeks | 918 | 1.2 | 1.1 | 40.0 | 0 |
| 5-8 | | | (10/910) | (1/7) | (0/1) |
| Months | 595 | 0.5 | 0.2 | 40.0 | 0 |
| 3-6 | | | (1/590) | (2/5) | (0/0) |
| Months | 595 | 0.2 | 1.6 | 0 | 0 |
| 6-12 | | | (9/551) | (0/5) | (0/0) |

Shaded cells depict ≥10% difference in AE recurrence in a given time frame relative to lower week 1 incidence group (e.g., light orange = occurred once > none; dark orange = occurred twice > occurred once).
In each cell, n/n represents the number of patients who experienced a recurrence of the given AE/number of patients who contributed data to the time period depicted in the row.

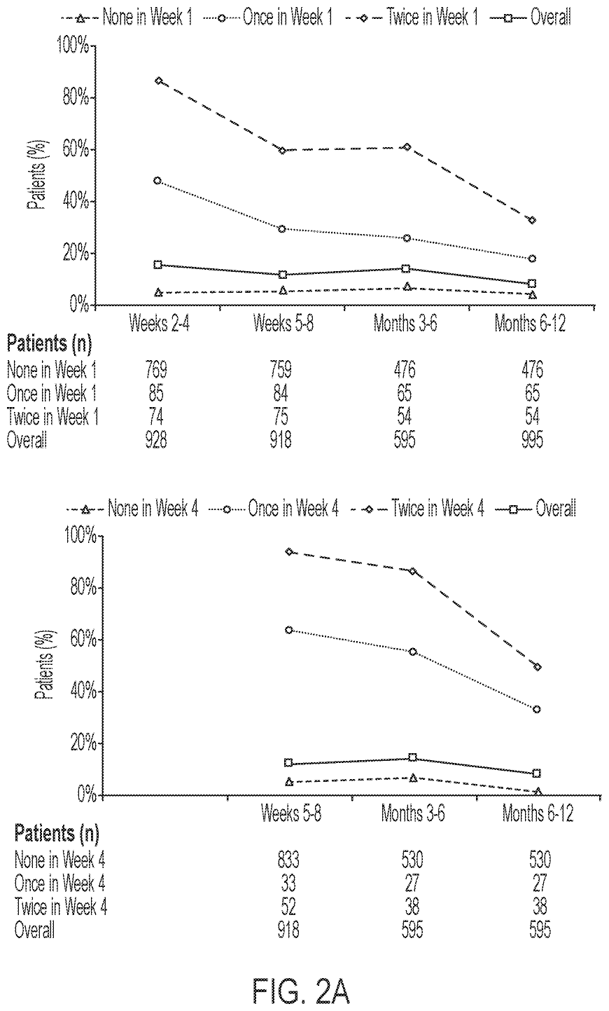

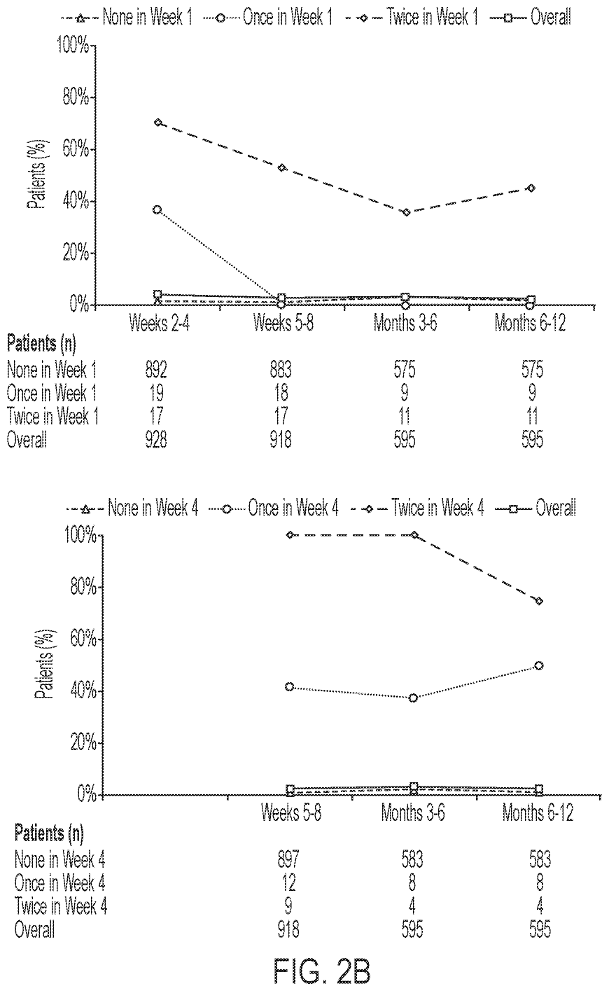

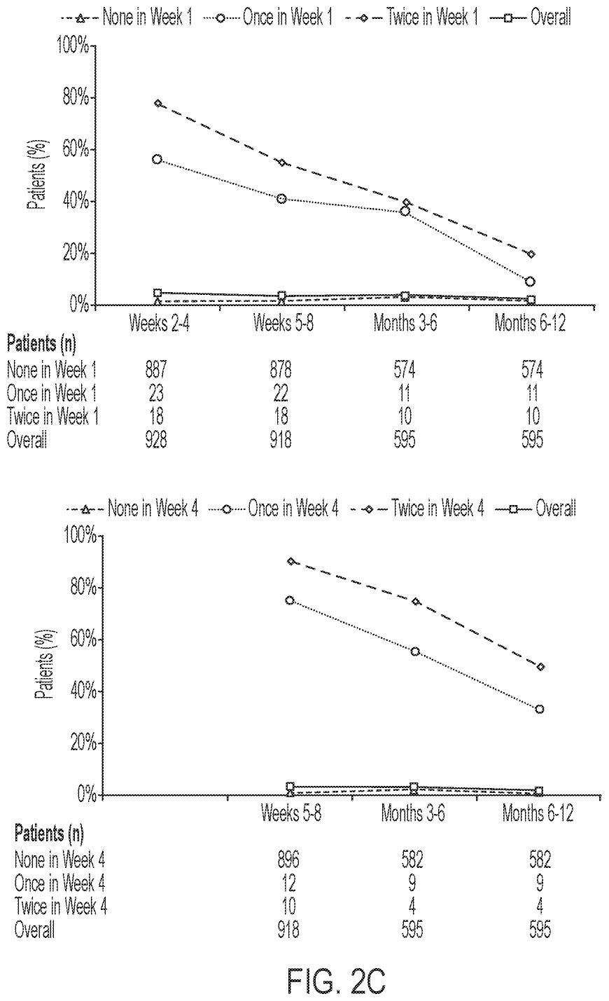

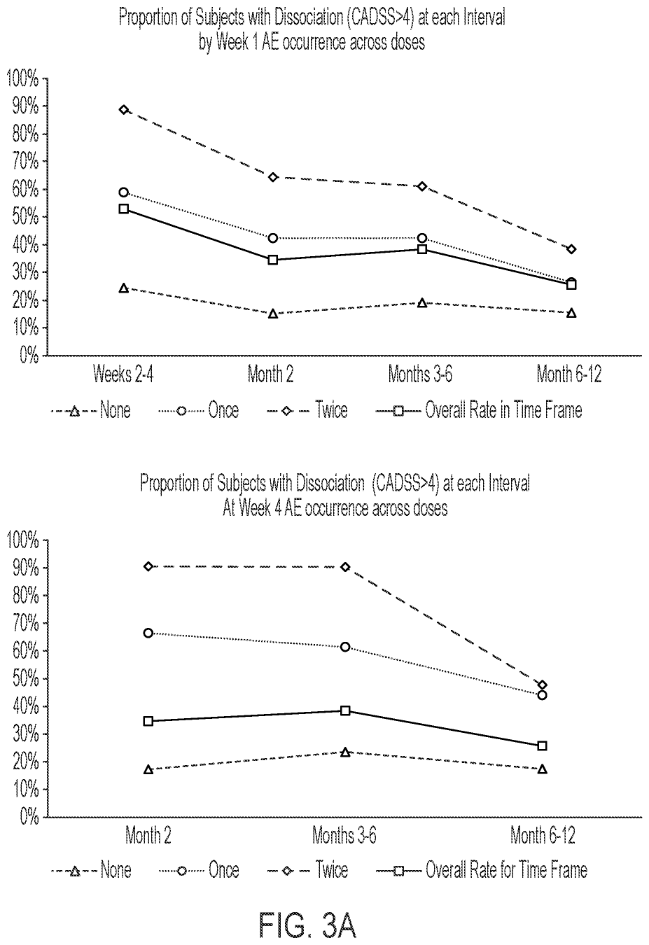

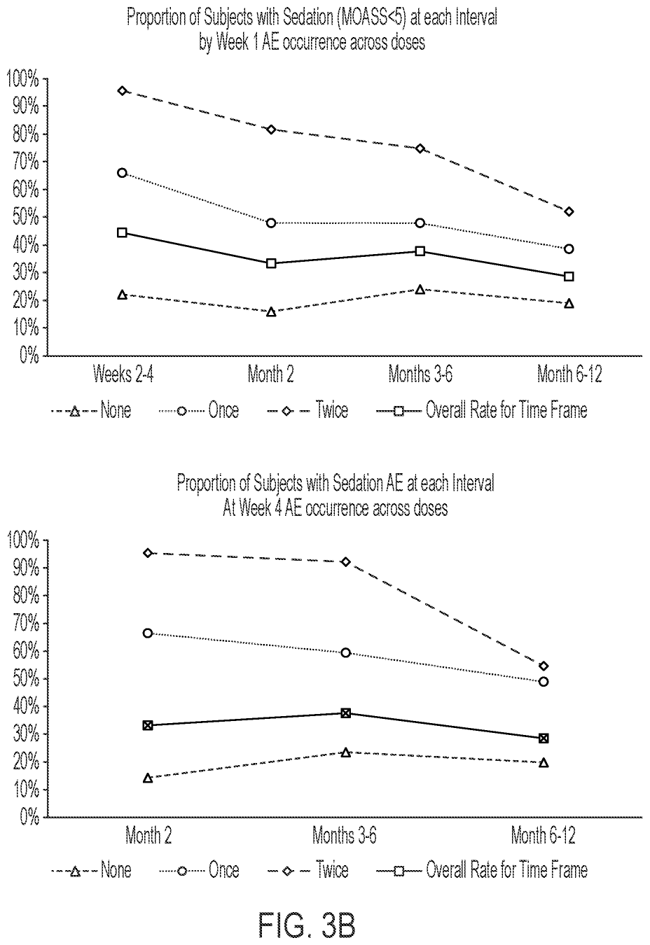

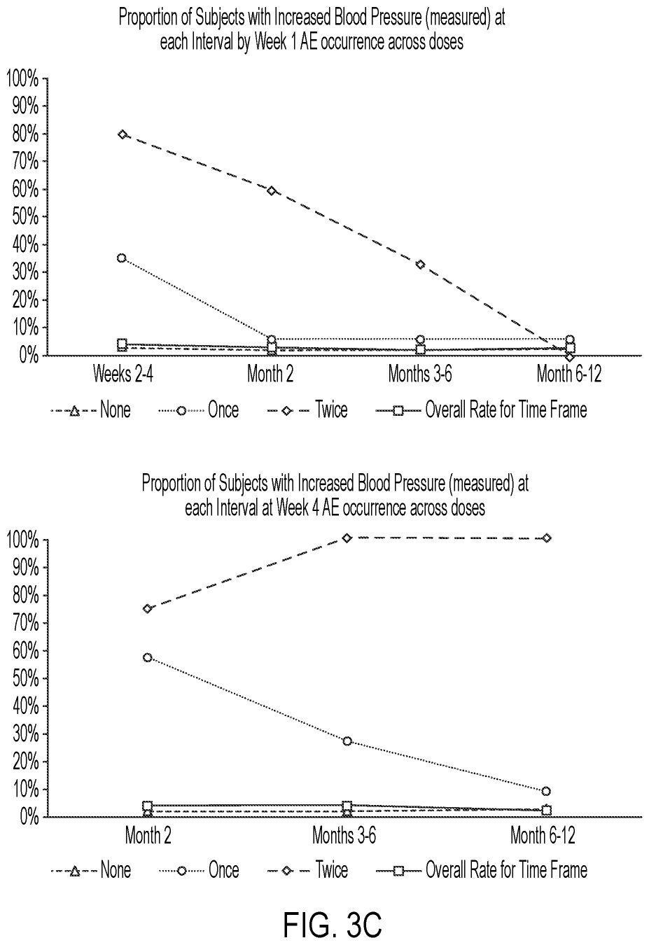

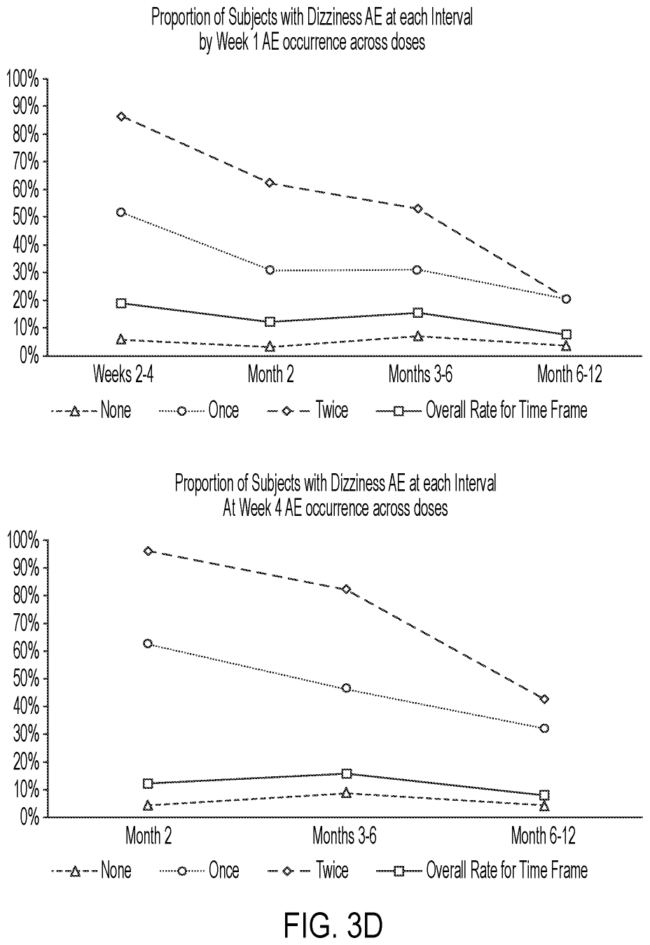

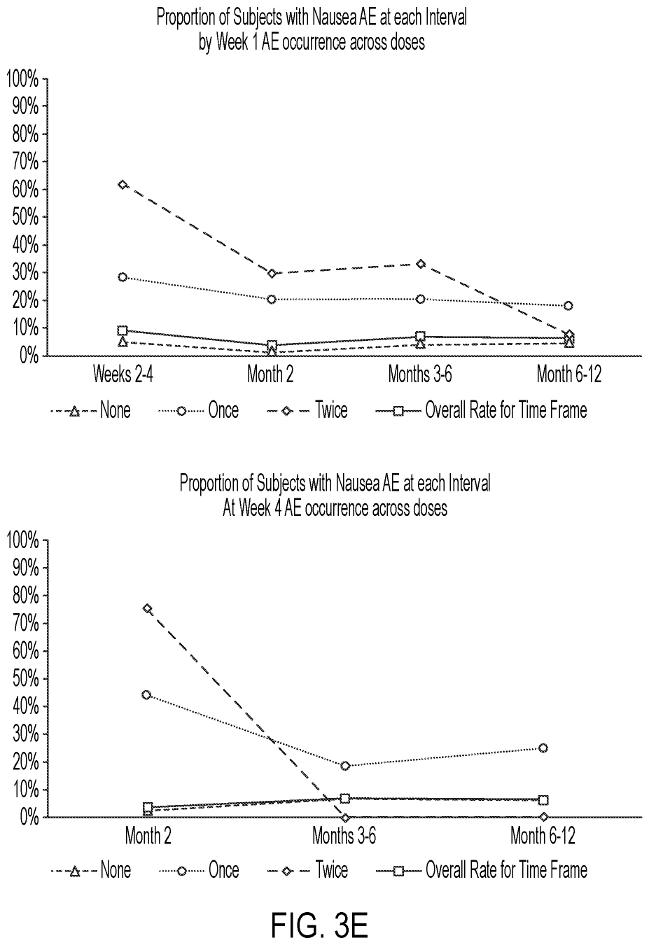

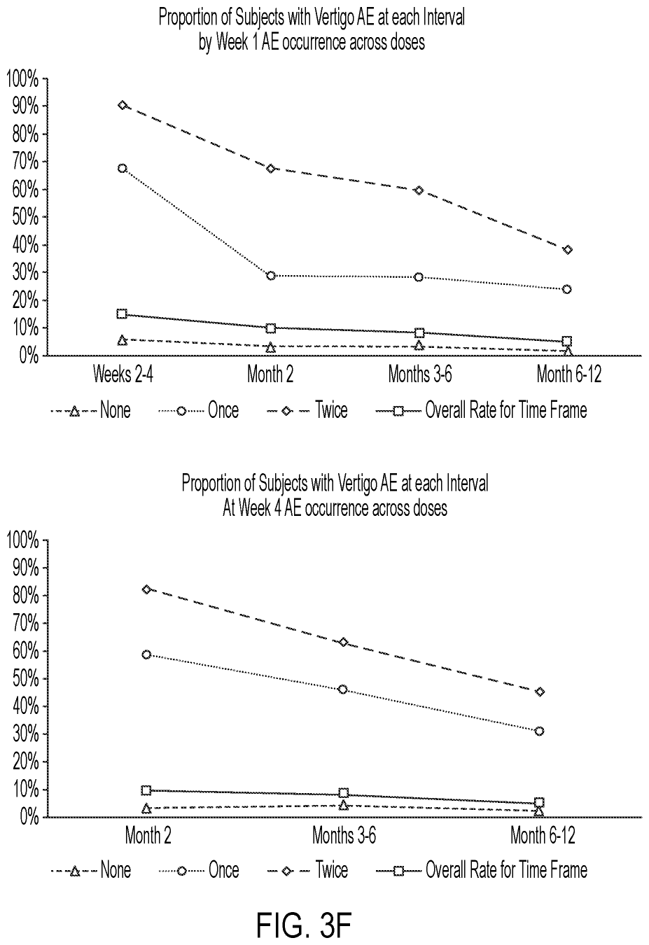

The rate of clinician-reported dissociation during Weeks 2-4 for the overall patient population was 16.1%, compared with 86.5% (64/74 patients) for those reporting dissociation twice in Week 1, 48.2% (41/85 patients) for those who reported dissociation once in Week 1, and 5.6% (44/790) for those who did not report dissociation at all in Week 1 (FIG. 2). The same general pattern (recurrence rates increasing as Week 1 frequency increases) was observed for each clinician-reported AE examined and for rates of dissociation, sedation, or elevated BP based on standardized measures (FIGS. 3A and 3B).

These results also provide insight into the size of the groups for whom AE recurrence was most or least likely to be observed. For instance, for all clinician-reported AEs, the group with the lowest rates of AE recurrence-those for whom an AE was not reported in Week 1—was the largest percentage of patients, making up a minimum of 78% of the sample (Table 3). These rates were lower for measure-based rates of dissociation and sedation (not reported during Week 1 in 43% and 62% of patients, respectively), which do not account for clinician perception of clinical significance.

### (a) Increased Blood Pressure

If increased blood pressure was reported as an AE in week 1, it was likely to recur during the induction period (weeks 2-4). Thereafter, increased blood pressure was more likely to recur at all time points in patients who experienced it twice in week 1 and was more likely to recur through months 3-6 in those who experienced it once in week 1. In those patients who did not have increased blood pressure during week 1 (95.7%), increased blood pressure was reported in <4% of patients during all subsequent postdose monitoring periods. The frequency of increased blood pressure during week 4 was more tightly linked to recurrence in later treatment sessions than frequency of increased blood pressure in week 1.

### (b) Nausea

Of those patients who did not report nausea during week 1 (86.0%), <6% reported nausea during subsequent postdose monitoring periods. The frequency of nausea in week 4 adds to the predictive power in month 2 but not thereafter.

### (c) Vomiting

Participants who experienced vomiting once during week 1 were more likely to have vomiting during subsequent periods: however, of the 8 participants who had vomiting twice during week 1, none experienced vomiting during later periods, despite only 1 of these participants receiving any relevant prophylactic or acute treatment.

### (d) Dissociation

Dissociative/perceptual changes include distortion of time and space and illusions, derealization, and depersonalization. Patients may describe these symptoms as feeling disconnected from themselves, their thoughts and feelings, and the things around them. Among patients who did not report dissociation during week 1 (83.2%), dissociation occurred in <10% for the remainder of the induction period. >50% of those who experienced dissociation twice in week 1 had dissociation as an AE into months 3-6. The frequency of AEs during week 4 appears to better predict the likelihood of recurrence of dissociation in later treatment sessions than week 1 incidence.

### (ii) Correlation Between the Severity of a Given AE Experienced Early in Treatment and the Severity of the AE Later in Treatment

The ability to test whether severity in Weeks 1 or 4 was predictive of subsequent severity was limited by the low number of patients for whom AEs were characterized as moderate or severe. For all clinician-reported AEs except dissociation, fewer than 5 patients had a severe recurrence during any of the observation periods following Week 1 or 4 occurrence. For clinician-reported AEs except dissociation, dizziness, vertigo and nausea, fewer than 10 patients experience a moderate recurrence during any of the observation periods following Week 1 or 4 occurrence.

Regardless of the highest reported severity of an AE during Weeks 1 or 4, recurrences tended to be mild to moderate in severity. There were no consistent differences between the average severities of recurrent AEs when comparing patients who did not report a given AE during Week 1 with those whose AE was mild. For clinician-reported dissociation, in patients whose AE was moderate or severe in Week 1, mean recurrence severity scores were 1.5 for both groups in Weeks 2-4, 1.3 and 1.8 in Weeks 5-8, 1.4 and 1.2 in Months 3-6, and 1.5 and 1.6 in Months 6-12, respectively (compared with 1.3 in all timeframes in patients without dissociation in Week 1).

In patients with a maximum CADSS total score≤14 (which generally responds to the mild-to-moderate range of reported AEs), there were no obvious differences in recurrence severity (mean individual scores 1.2-1.4 across the different follow-up timeframes, where 1=mild and 2=moderate). The mean individual CADSS scores in patients with maximum CADSS total scores 15-24 and 24-42 were 1.3-2.1 across the different follow-up timeframes, and <3 patients in had a maximum CADSS score >42. Regardless of highest reported MOAA/S during either Week 1 or Week 4 (minimum score-0) [indicating most severe sedation]: maximum score 5 [no sedation]), average recurrences thereafter were mild (mean scores 3.5-4.0, except for a single patient with a mean recurrence score of 3.25).

(iii) Comparison of the Incidence of AEs at the End of the Induction Period (Week 4) Vs. Week 1 on Indication of Recurrence Incidence or Severity

AE recurrence after Week 4 was more closely linked to Week 4 AE frequency than Week 1 AE frequency. When an AE did not occur in either Week 1 or Week 4, there was little difference between the Weeks in terms of their prognostic utility. This is shown for clinician-reported dissociation, sedation, and increased BP in FIG. 2. Relationships for other AEs follow a similar pattern (FIGS. 3A-3F) including measure-based rates of dissociation, sedation, and increased BP, though the differences in the latter were relatively small.

### (iv) Impact of Dosing and Concomitant Medications

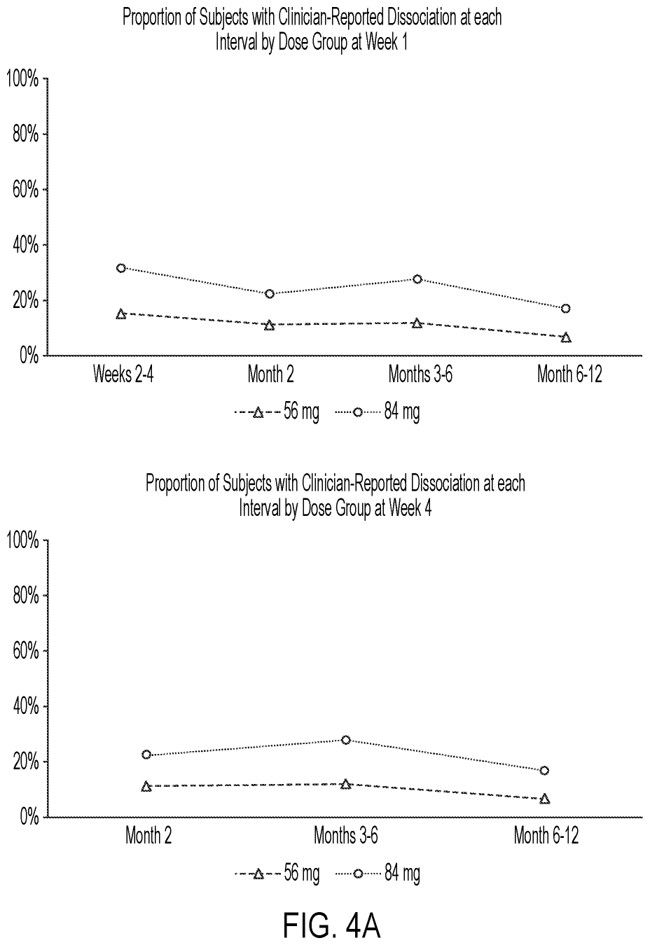

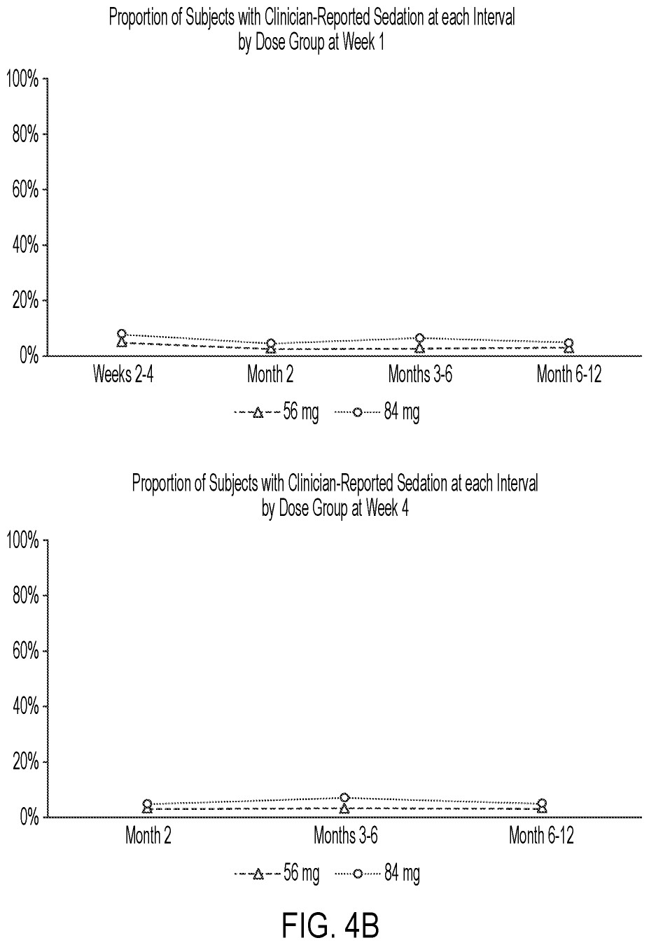

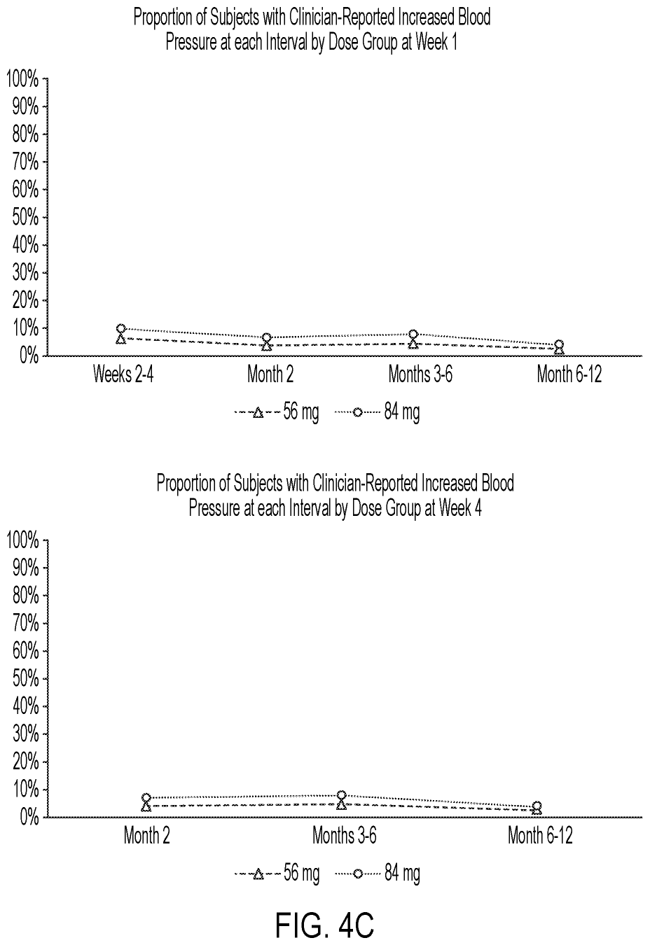

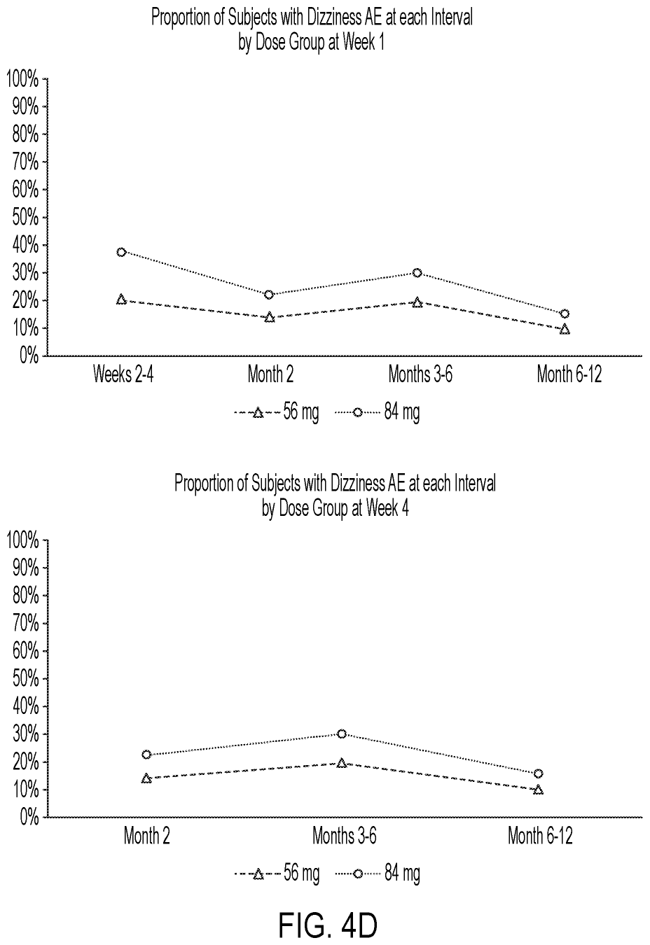

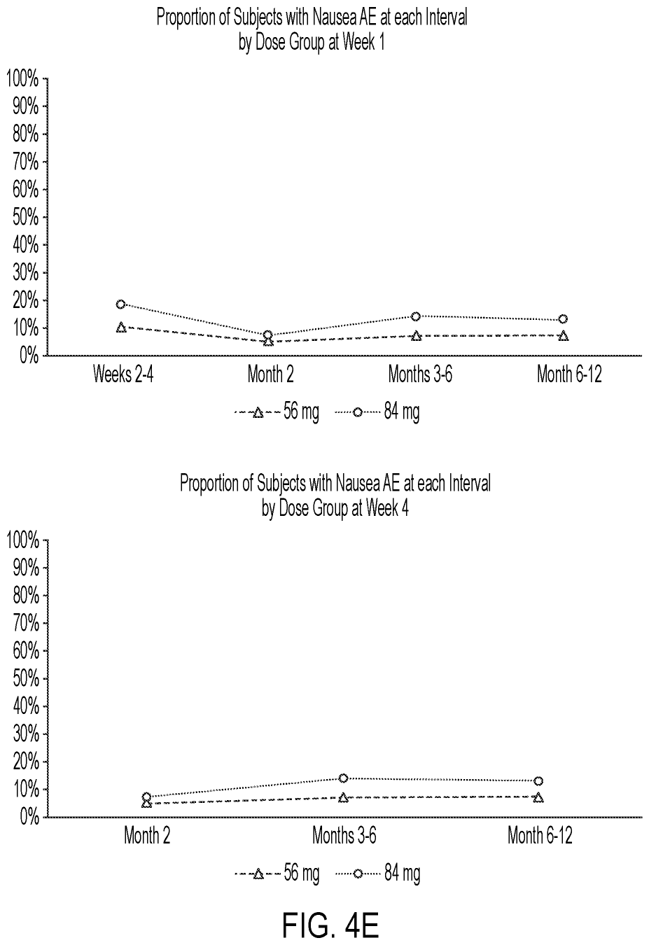

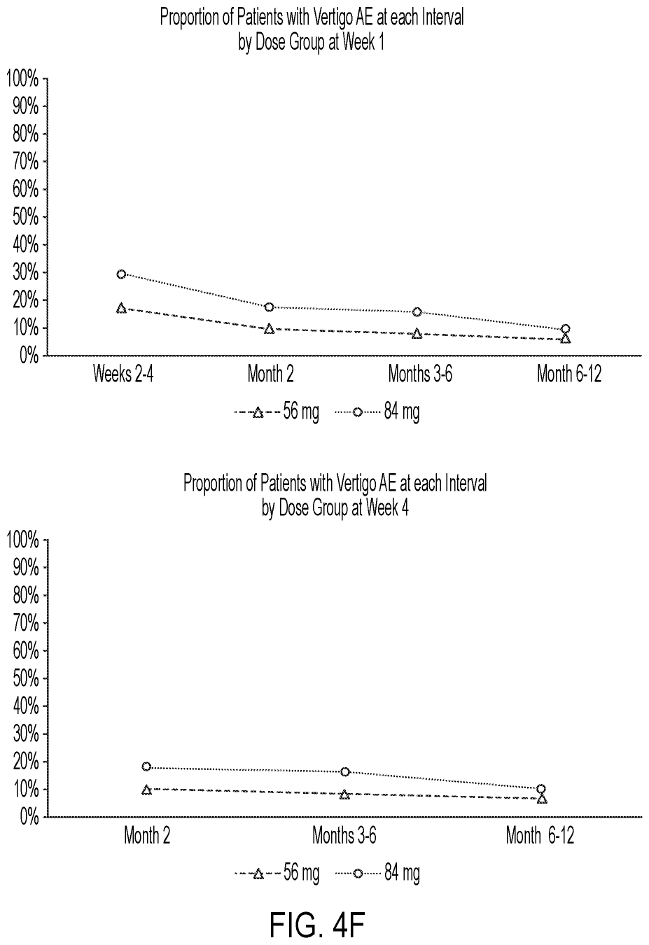

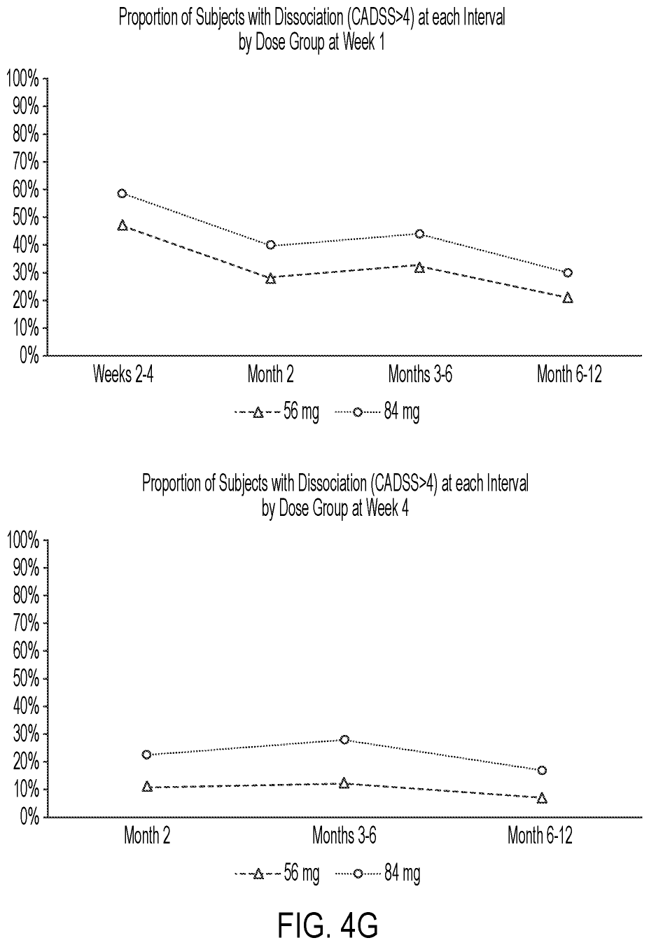

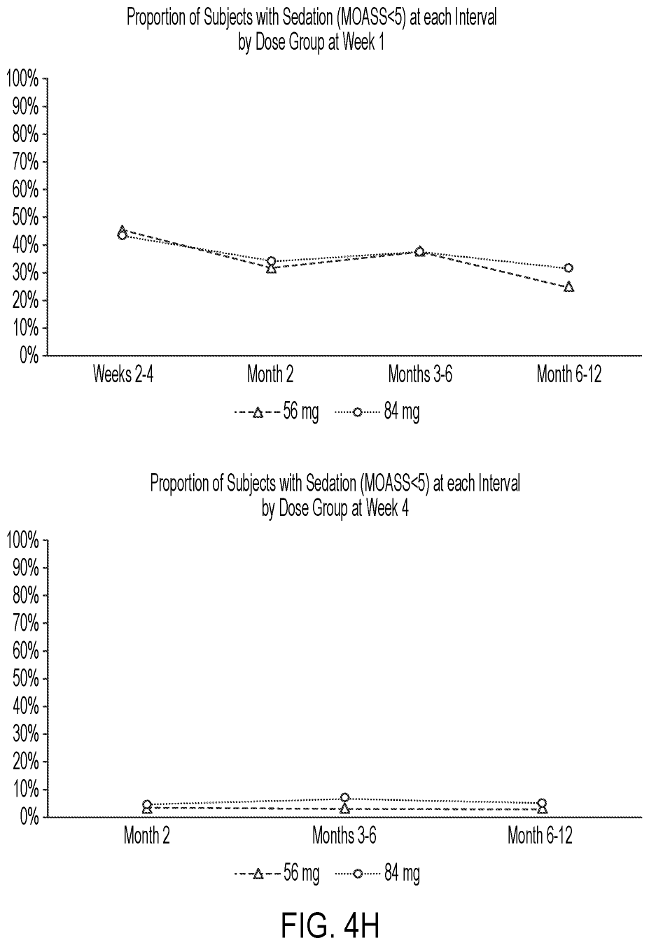

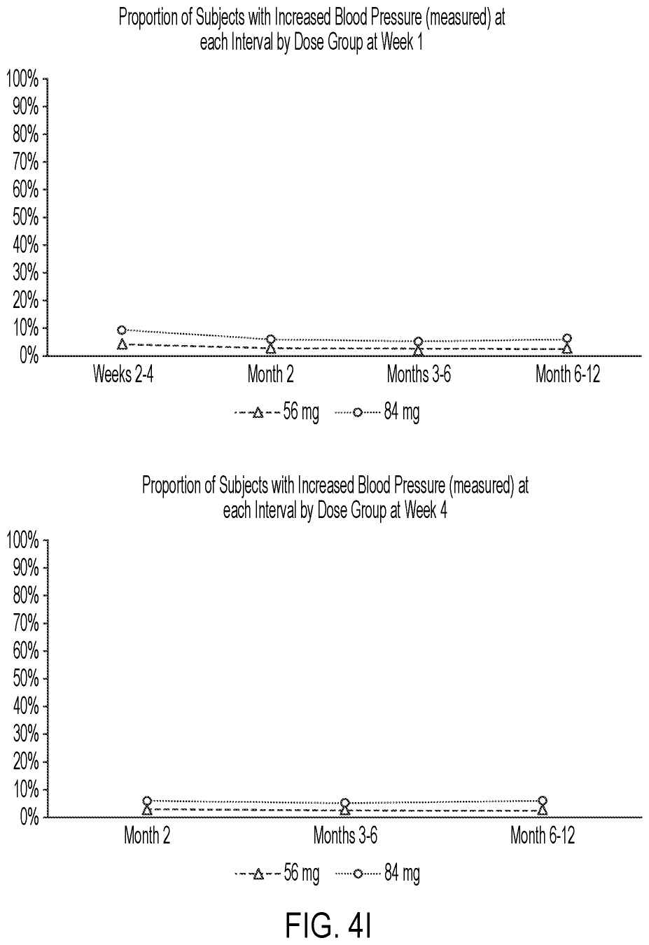

Since flexible dosing (ESK 56 or 84 mg) was permitted in the studies and an individual patient's doses could change within dosing timeframes, patients were stratified by modal dose during the respective periods. A small dose effect was most pronounced with dissociation and dizziness (FIGS. 4A-4I), but the impact of dose on AE recurrence rates was of much smaller magnitude than the effect of Week 1 AE frequency.

Proactive or symptomatic management of potential or observed AEs likely had minimal impact on the observed patterns since no more than four patients received prophylactic or symptomatic treatment for the respective AEs during the respective timeframes (Table 4).

TABLE 4
Patients receiving concomitant medications for clinician-reported adverse events

| | | | | | | | Increased |
|---|---|---|---|---|---|---|---|
| Patients, n (%)^{a} | Dizziness | Dissociation | Sedation | Vertigo | Nausea | | blood pressure |
| Weeks 2-4 | | | | | | | |
| Prophylactic | 0 | 2/159 (1.3%) | 0 | 0 | 0 | | 1/41 (2.4%) |
| Symptomatic | 2/196 (1%) | 0 | 0 | 0 | 4/133 | (3%) | 2/41 (4.9%) |
| Weeks 5-8 | | | | | | | |
| Prophylactic | 0 | 2/159 (1.3%) | 0 | 0 | 0 | | 1/40 (2.5%) |
| Symptomatic | 0 | 2/159 (1.3%) | 0 | 0 | 1/132 | (0.8%) | 2/40 (5.0%) |
| Months 3-6 | | | | | | | |
| Prophylactic | 0 | 2/119 (1.7%) | 0 | 0 | 0 | | 0 |
| Symptomatic | 0 | 2/119 (1.7%) | 0 | 0 | 3/89 | (3.4%) | 0 |
| Months 6-12 | | | | | | | |
| Prophylactic | 0 | 2/119 (1.7%) | 0 | 0 | 0 | | 0 |
| Symptomatic | 0 | 1/119 (0.8%) | 0 | 0 | 1/89 | (1.1%) | 0 |

^{a}Denominator is number of patients experiencing the adverse event in the respective timeframe

For dissociation, across the duration of the trial, only three patients received symptomatic medication (alprazolam, lorazepam), whereas two patients received prophylactic medication across every interval (lorazepam, diazepam). For blood pressure, two patients received symptomatic treatment in Weeks 2-4 (losartan, captopril), while two others received symptomatic treatment in Weeks 5-8 (ramipril, enalapril). No patient received symptomatic medication for blood pressure in more than one time period, and no symptomatic medication for blood pressure was administered after Week 8. One patient was given propranolol prophylactically during Weeks 2-4 and Weeks 5-8. Four patients received medication for nausea in Weeks 2-4 (ondansetron), one of whom received medicated in two later time periods, and two of whom each received symptomatic medication in one additional time period. Two patients received symptomatic medication for dizziness (betahistine) during Weeks 2-4; no patients received medication for dizziness thereafter.

### (v) Time to Onset of Adverse Events

The time to onset of the adverse events experienced by patients in Example 2 was analyzed. Tables 5-11 include the summary of specific adverse events by their respective categorized maximum time to Onset (i) across sessions 3-8 during Induction Phase by AE week 1 occurrence (Table 5), (ii) during Optimization Phase by AE week 1 occurrence (Table 6), (iii) during Optimization Phase by AE week 4 occurrence (Table 7), (iv) during Month 3-6 of Maintenance Phase by AE week 1 occurrence (Table 8), (v) Month 3-6 of Maintenance Phase by AE week 4 occurrence (Table 9), (vi) Month 6-12 of Maintenance Phase by AE week 1 occurrence (Table 10), and (vii) during Month 6-12 of Maintenance Phase by AE week 4 (Table 11).

TABLE 5
Summary of specific adverse events (Binary) by their
respective categorized maximum time to Onset across
sessions 3-8 during Induction Phase by AE week 1
occurrence (Studies TRD3003 and TRD3004 and Placebo
patients of maintenance phase of study 3003)

| | modal dose | modal dose | |
|---|---|---|---|
| | 56 | 84 | Total |
| Blood pressure increased | | | |
| (clinician-reported) | | | |
| AE week 1 occurrence/ | | | |
| Time to Onset in sessions | | | |
| 3-8 of the Induction Phase | | | |
| N | 29 (13.3%) | 19 (10.1%) | 48 (11.8%) |
| None | 11 (5.0%) | 10 (5.3%) | 21 (5.2%) |
| 31-60 minutes | 10 (90.9%) | 10 (100%) | 20 (95.2%) |
| 61-90 minutes | 1 (9.1%) | 0 (0.0%) | 1 (4.8%) |
| Once | 8 (3.7%) | 5 (2.6%) | 13 (3.2%) |
| 0-30 minutes | 1 (12.5%) | 0 (0.0%) | 1 (7.7%) |
| 31-60 minutes | 6 (75.0%) | 5 (100%) | 11 (84.6%) |
| 61-90 minutes | 1 (12.5%) | 0 (0.0%) | 1 (7.7%) |
| Twice | 10 (4.6%) | 4 (2.1%) | 14 (3.4%) |
| 31-60 minutes | 9 (90.0%) | 3 (75.0%) | 12 (85.7%) |
| 61-90 minutes | 1 (10.0%) | 0 (0.0%) | 1 (7.1%) |
| >90 minutes | 0 (0.0%) | 1 (25.0%) | 1 (7.1%) |
| Dissociation | | | |
| (clinician-reported) | | | |
| AE week 1 occurrence/ | | | |
| Time to Onset in sessions | | | |
| 3-8 of the Induction Phase | | | |
| N | 69 (31.7%) | 80 (42.3%) | 149 (36.6%) |
| None | 17 (7.8%) | 27 (14.3%) | 44 (10.8%) |
| 0-30 minutes | 15 (88.2%) | 19 (70.4%) | 34 (77.3%) |
| 31-60 minutes | 1 (5.9%) | 7 (25.9%) | 8 (18.2%) |
| missing | 1 (5.9%) | 1 (3.7%) | 2 (4.5%) |
| Once | 16 (7.3%) | 25 (13.2%) | 41 (10.1%) |
| 0-30 minutes | 13 (81.3%) | 16 (64.0%) | 29 (70.7%) |
| 31-60 minutes | 1 (6.3%) | 8 (32.0%) | 9 (22.0%) |
| 61-90 minutes | 0 (0.0%) | 1 (4.0%) | 1 (2.4%) |
| >90 minutes | 2 (12.5%) | 0 (0.0%) | 2 (4.9%) |
| Twice | 36 (16.5%) | 28 (14.8%) | 64 (15.7%) |
| 0-30 minutes | 27 (75.0%) | 17 (60.7%) | 44 (68.8%) |
| 31-60 minutes | 7 (19.4%) | 9 (32.1%) | 16 (25.0%) |
| 61-90 minutes | 2 (5.6%) | 2 (7.1%) | 4 (6.3%) |
| Dizziness | | | |
| (clinician-reported) | | | |
| AE week 1 occurrence/ | | | |
| Time to Onset in sessions | | | |
| 3-8 of the Induction Phase | | | |
| N | 93 (42.7%) | 85 (45.0%) | 178 (43.7%) |
| None | 24 (11.0%) | 22 (11.6%) | 46 (11.3%) |
| 0-30 minutes | 19 (79.2%) | 16 (72.7%) | 35 (76.1%) |
| 31-60 minutes | 2 (8.3%) | 3 (13.6%) | 5 (10.9%) |
| 61-90 minutes | 0 (0.0%) | 2 (9.1%) | 2 (4.3%) |
| missing | 3 (12.5%) | 1 (4.5%) | 4 (8.7%) |
| Once | 33 (15.1%) | 23 (12.2%) | 56 (13.8%) |
| 0-30 minutes | 25 (75.8%) | 16 (69.6%) | 41 (73.2%) |
| 31-60 minutes | 4 (12.1%) | 4 (17.4%) | 8 (14.3%) |
| 61-90 minutes | 1 (3.0%) | 1 (4.3%) | 2 (3.6%) |
| >90 minutes | 1 (3.0%) | 0 (0.0%) | 1 (1.8%) |
| missing | 2 (6.1%) | 2 (8.7%) | 4 (7.1%) |
| Twice | 36 (16.5%) | 40 (21.2%) | 76 (18.7%) |
| 0-30 minutes | 32 (88.9%) | 28 (70.0%) | 60 (78.9%) |
| 31-60 minutes | 1 (2.8%) | 8 (20.0%) | 9 (11.8%) |
| 61-90 minutes | 1 (2.8%) | 3 (7.5%) | 4 (5.3%) |
| >90 minutes | 1 (2.8%) | 1 (2.5%) | 2 (2.6%) |
| missing | 1 (2.8%) | 0 (0.0%) | 1 (1.3%) |
| Sedation | | | |
| (clinician-reported) | | | |
| AE week 1 occurrence/ | | | |
| Time to Onset in sessions | | | |
| 3-8 of the Induction Phase | | | |
| N | 24 (11.0%) | 14 (7.4%) | 38 (9.3%) |
| None | 9 (4.1%) | 10 (5.3%) | 19 (4.7%) |
| 0-30 minutes | 4 (44.4%) | 5 (50.0%) | 9 (47.4%) |
| 31-60 minutes | 2 (22.2%) | 2 (20.0%) | 4 (21.1%) |
| 61-90 minutes | 1 (11.1%) | 0 (0.0%) | 1 (5.3%) |
| >90 minutes | 1 (11.1%) | 1 (10.0%) | 2 (10.5%) |
| missing | 1 (11.1%) | 2 (20.0%) | 3 (15.8%) |
| Once | 7 (3.2%) | 0 | 7 (1.7%) |
| 0-30 minutes | 1 (14.3%) | 0 | 1 (14.3%) |
| 31-60 minutes | 1 (14.3%) | 0 | 1 (14.3%) |
| 61-90 minutes | 3 (42.9%) | 0 | 3 (42.9%) |
| >90 minutes | 1 (14.3%) | 0 | 1 (14.3%) |
| missing | 1 (14.3%) | 0 | 1 (14.3%) |
| Twice | 8 (3.7%) | 4 (2.1%) | 12 (2.9%) |
| 0-30 minutes | 5 (62.5%) | 2 (50.0%) | 7 (58.3%) |
| 31-60 minutes | 0 (0.0%) | 2 (50.0%) | 2 (16.7%) |
| 61-90 minutes | 2 (25.0%) | 0 (0.0%) | 2 (16.7%) |
| missing | 1 (12.5%) | 0 (0.0%) | 1 (8.3%) |
| Vertigo | | | |
| (clinician-reported) | | | |
| AE week 1 occurrence/ | | | |
| Time to Onset in sessions | | | |
| 3-8 of the Induction Phase | | | |
| N | 79 (36.2%) | 60 (31.7%) | 139 (34.2%) |
| None | 30 (13.8%) | 15 (7.9%) | 45 (11.1%) |
| 0-30 minutes | 20 (66.7%) | 11 (73.3%) | 31 (68.9%) |
| 31-60 minutes | 6 (20.0%) | 1 (6.7%) | 7 (15.6%) |
| 61-90 minutes | 1 (3.3%) | 1 (6.7%) | 2 (4.4%) |
| >90 minutes | 1 (3.3%) | 2 (13.3%) | 3 (6.7%) |
| missing | 2 (6.7%) | 0 (0.0%) | 2 (4.4%) |
| Once | 11 (5.0%) | 18 (9.5%) | 29 (7.1%) |
| 0-30 minutes | 8 (72.7%) | 11 (61.1%) | 19 (65.5%) |
| 31-60 minutes | 2 (18.2%) | 5 (27.8%) | 7 (24.1%) |
| 61-90 minutes | 0 (0.0%) | 1 (5.6%) | 1 (3.4%) |
| >90 minutes | 1 (9.1%) | 1 (5.6%) | 2 (6.9%) |
| Twice | 38 (17.4%) | 27 (14.3%) | 65 (16.0%) |
| 0-30 minutes | 34 (89.5%) | 23 (85.2%) | 57 (87.7%) |
| 31-60 minutes | 3 (7.9%) | 2 (7.4%) | 5 (7.7%) |
| 61-90 minutes | 1 (2.6%) | 0 (0.0%) | 1 (1.5%) |
| >90 minutes | 0 (0.0%) | 2 (7.4%) | 2 (3.1%) |

TABLE 6
Summary of specific adverse events (Binary) by their
respective categorized maximum time to Onset during
Optimization Phase by AE week 1 occurrence (Studies TRD3003 and
TRD3004 and Placebo patients of maintenance phase of study 3003)

| | modal dose | modal dose | |
|---|---|---|---|
| | 56 | 84 | Total |
| Blood pressure increased | | | |
| (clinician-reported) | | | |
| AE week 1 occurrence | | | |
| N | 16 (12.1%) | 20 (12.0%) | 36 (12.0%) |
| None | 7 (5.3%) | 10 (6.0%) | 17 (5.7%) |
| 0-30 minutes | 1 (14.3%) | 0 (0.0%) | 1 (5.9%) |
| 31-60 minutes | 4 (57.1%) | 8 (80.0%) | 12 (70.6%) |
| 61-90 minutes | 0 (0.0%) | 2 (20.0%) | 2 (11.8%) |
| missing | 2 (28.6%) | 0 (0.0%) | 2 (11.8%) |
| Once | 2 (1.5%) | 7 (4.2%) | 9 (3.0%) |
| 31-60 minutes | 2 (100%) | 7 (100%) | 9 (100%) |
| Twice | 7 (5.3%) | 3 (1.8%) | 10 (3.3%) |
| 31-60 minutes | 7 (100%) | 3 (100%) | 10 (100%) |
| Dissociation | | | |
| (clinician-reported) | | | |
| AE week 1 occurrence | | | |
| N | 44 (33.3%) | 72 (43.1%) | 116 (38.8%) |
| None | 24 (18.2%) | 22 (13.2%) | 46 (15.4%) |
| 0-30 minutes | 19 (79.2%) | 14 (63.6%) | 33 (71.7%) |
| 31-60 minutes | 3 (12.5%) | 5 (22.7%) | 8 (17.4%) |
| missing | 2 (8.3%) | 3 (13.6%) | 5 (10.9%) |
| Once | 5 (3.8%) | 20 (12.0%) | 25 (8.4%) |
| 0-30 minutes | 5 (100%) | 16 (80.0%) | 21 (84.0%) |
| 31-60 minutes | 0 (0.0%) | 3 (15.0%) | 3 (12.0%) |
| 61-90 minutes | 0 (0.0%) | 1 (5.0%) | 1 (4.0%) |
| Twice | 15 (11.4%) | 30 (18.0%) | 45 (15.1%) |
| 0-30 minutes | 13 (86.7%) | 22 (73.3%) | 35 (77.8%) |
| 31-60 minutes | 2 (13.3%) | 8 (26.7%) | 10 (22.2%) |
| Dizziness | | | |
| (clinician-reported) | | | |
| AE week 1 occurrence | | | |
| N | 55 (41.7%) | 60 (35.9%) | 115 (38.5%) |
| None | 11 (8.3%) | 16 (9.6%) | 27 (9.0%) |
| 0-30 minutes | 10 (90.9%) | 11 (68.8%) | 21 (77.8%) |
| 31-60 minutes | 0 (0.0%) | 2 (12.5%) | 2 (7.4%) |
| 61-90 minutes | 0 (0.0%) | 1 (6.3%) | 1 (3.7%) |
| missing | 1 (9.1%) | 2 (12.5%) | 3 (11.1%) |
| Once | 18 (13.6%) | 15 (9.0%) | 33 (11.0%) |
| 0-30 minutes | 14 (77.8%) | 13 (86.7%) | 27 (81.8%) |
| 31-60 minutes | 2 (11.1%) | 0 (0.0%) | 2 (6.1%) |
| >90 minutes | 1 (5.6%) | 1 (6.7%) | 2 (6.1%) |
| missing | 1 (5.6%) | 1 (6.7%) | 2 (6.1%) |
| Twice | 26 (19.7%) | 29 (17.4%) | 55 (18.4%) |
| 0-30 minutes | 25 (96.2%) | 24 (82.8%) | 49 (89.1%) |
| 31-60 minutes | 0 (0.0%) | 2 (6.9%) | 2 (3.6%) |
| 61-90 minutes | 0 (0.0%) | 3 (10.3%) | 3 (5.5%) |
| missing | 1 (3.8%) | 0 (0.0%) | 1 (1.8%) |
| Sedation | | | |
| (clinician-reported) | | | |
| AE week 1 occurrence | | | |
| N | 12 (9.1%) | 12 (7.2%) | 24 (8.0%) |
| None | 4 (3.0%) | 7 (4.2%) | 11 (3.7%) |
| 0-30 minutes | 3 (75.0%) | 6 (85.7%) | 9 (81.8%) |
| missing | 1 (25.0%) | 1 (14.3%) | 2 (18.2%) |
| Once | 2 (1.5%) | 2 (1.2%) | 4 (1.3%) |
| 0-30 minutes | 1 (50.0%) | 2 (100%) | 3 (75.0%) |
| 31-60 minutes | 1 (50.0%) | 0 (0.0%) | 1 (25.0%) |
| Twice | 6 (4.5%) | 3 (1.8%) | 9 (3.0%) |
| 0-30 minutes | 6 (100%) | 1 (33.3%) | 7 (77.8%) |
| 31-60 minutes | 0 (0.0%) | 2 (66.7%) | 2 (22.2%) |
| Vertigo | | | |
| (clinician-reported) | | | |
| AE week 1 occurrence | | | |
| N | 39 (29.5%) | 52 (31.1%) | 91 (30.4%) |
| None | 9 (6.8%) | 15 (9.0%) | 24 (8.0%) |
| 0-30 minutes | 7 (77.8%) | 11 (73.3%) | 18 (75.0%) |
| 31-60 minutes | 0 (0.0%) | 2 (13.3%) | 2 (8.3%) |
| 61-90 minutes | 0 (0.0%) | 1 (6.7%) | 1 (4.2%) |
| missing | 2 (22.2%) | 1 (6.7%) | 3 (12.5%) |
| Once | 8 (6.1%) | 11 (6.6%) | 19 (6.4%) |
| 0-30 minutes | 5 (62.5%) | 9 (81.8%) | 14 (73.7%) |
| 31-60 minutes | 1 (12.5%) | 1 (9.1%) | 2 (10.5%) |
| 61-90 minutes | 1 (12.5%) | 0 (0.0%) | 1 (5.3%) |
| >90 minutes | 1 (12.5%) | 1 (9.1%) | 2 (10.5%) |
| Twice | 22 (16.7%) | 26 (15.6%) | 48 (16.1%) |
| 0-30 minutes | 22 (100%) | 24 (92.3%) | 46 (95.8%) |
| 31-60 minutes | 0 (0.0%) | 2 (7.7%) | 2 (4.2%) |

TABLE 7
Summary of specific adverse events (Binary) by their respective
categorized maximum time to Onset during Optimization
Phase by AE week 4 occurrence (Studies TRD3003 and
TRD3004 and Placebo patients of maintenance phase of study 3003)

| | modal dose | modal dose | |
|---|---|---|---|
| | 56 | 84 | Total |
| Blood pressure increased | | | |
| (clinician-reported) | | | |
| AE week 4 occurrence | | | |
| N | 16 (12.1%) | 20 (12.0%) | 36 (12.0%) |
| None | 9 (6.8%) | 9 (5.4%) | 18 (6.0%) |
| 0-30 minutes | 1 (11.1%) | 0 (0.0%) | 1 (5.6%) |
| 31-60 minutes | 6 (66.7%) | 7 (77.8%) | 13 (72.2%) |
| 61-90 minutes | 0 (0.0%) | 2 (22.2%) | 2 (11.1%) |
| missing | 2 (22.2%) | 0 (0.0%) | 2 (11.1%) |
| Once | 2 (1.5%) | 7 (4.2%) | 9 (3.0%) |
| 31-60 minutes | 2 (100%) | 7 (100%) | 9 (100%) |
| Twice | 5 (3.8%) | 4 (2.4%) | 9 (3.0%) |
| 31-60 minutes | 5 (100%) | 4 (100%) | 9 (100%) |
| Dissociation | | | |
| (clinician-reported) | | | |
| AE week 4 occurrence | | | |
| N | 44 (33.3%) | 72 (43.1%) | 116 (38.8%) |
| None | 23 (17.4%) | 23 (13.8%) | 46 (15.4%) |
| 0-30 minutes | 18 (78.3%) | 15 (65.2%) | 33 (71.7%) |
| 31-60 minutes | 3 (13.0%) | 5 (21.7%) | 8 (17.4%) |
| missing | 2 (8.7%) | 3 (13.0%) | 5 (10.9%) |
| Once | 7 (5.3%) | 14 (8.4%) | 21 (7.0%) |
| 0-30 minutes | 7 (100%) | 11 (78.6%) | 18 (85.7%) |
| 31-60 minutes | 0 (0.0%) | 2 (14.3%) | 2 (9.5%) |
| 61-90 minutes | 0 (0.0%) | 1 (7.1%) | 1 (4.8%) |
| Twice | 14 (10.6%) | 35 (21.0%) | 49 (16.4%) |
| 0-30 minutes | 12 (85.7%) | 26 (74.3%) | 38 (77.6%) |
| 31-60 minutes | 2 (14.3%) | 9 (25.7%) | 11 (22.4%) |
| Dizziness | | | |
| (clinician-reported) | | | |
| AE week 4 occurrence | | | |
| N | 55 (41.7%) | 60 (35.9%) | 115 (38.5%) |
| None | 17 (12.9%) | 18 (10.8%) | 35 (11.7%) |
| 0-30 minutes | 14 (82.4%) | 12 (66.7%) | 26 (74.3%) |
| 31-60 minutes | 0 (0.0%) | 2 (11.1%) | 2 (5.7%) |
| 61-90 minutes | 0 (0.0%) | 2 (11.1%) | 2 (5.7%) |
| >90 minutes | 1 (5.9%) | 0 (0.0%) | 1 (2.9%) |
| missing | 2 (11.8%) | 2 (11.1%) | 4 (11.4%) |
| Once | 11 (8.3%) | 16 (9.6%) | 27 (9.0%) |
| 0-30 minutes | 10 (90.9%) | 14 (87.5%) | 24 (88.9%) |
| 31-60 minutes | 1 (9.1%) | 1 (6.3%) | 2 (7.4%) |
| 61-90 minutes | 0 (0.0%) | 1 (6.3%) | 1 (3.7%) |
| Twice | 27 (20.5%) | 26 (15.6%) | 53 (17.7%) |
| 0-30 minutes | 25 (92.6%) | 22 (84.6%) | 47 (88.7%) |
| 31-60 minutes | 1 (3.7%) | 1 (3.8%) | 2 (3.8%) |
| 61-90 minutes | 0 (0.0%) | 1 (3.8%) | 1 (1.9%) |
| >90 minutes | 0 (0.0%) | 1 (3.8%) | 1 (1.9%) |
| missing | 1 (3.7%) | 1 (3.8%) | 2 (3.8%) |
| Sedation | | | |
| (clinician-reported) | | | |
| AE week 4 occurrence | | | |
| N | 12 (9.1%) | 12 (7.2%) | 24 (8.0%) |
| None | 5 (3.8%) | 5 (3.0%) | 10 (3.3%) |
| 0-30 minutes | 4 (80.0%) | 5 (100%) | 9 (90.0%) |
| missing | 1 (20.0%) | 0 (0.0%) | 1 (10.0%) |
| Once | 2 (1.5%) | 3 (1.8%) | 5 (1.7%) |
| 0-30 minutes | 2 (100%) | 3 (100%) | 5 (100%) |
| Twice | 5 (3.8%) | 4 (2.4%) | 9 (3.0%) |
| 0-30 minutes | 4 (80.0%) | 1 (25.0%) | 5 (55.6%) |
| 31-60 minutes | 1 (20.0%) | 2 (50.0%) | 3 (33.3%) |
| missing | 0 (0.0%) | 1 (25.0%) | 1 (11.1%) |
| Vertigo | | | |
| (clinician-reported) | | | |
| AE week 4 occurrence | | | |
| N | 39 (29.5%) | 52 (31.1%) | 91 (30.4%) |
| None | 10 (7.6%) | 15 (9.0%) | 25 (8.4%) |
| 0-30 minutes | 6 (60.0%) | 12 (80.0%) | 18 (72.0%) |
| 31-60 minutes | 0 (0.0%) | 1 (6.7%) | 1 (4.0%) |
| 61-90 minutes | 1 (10.0%) | 1 (6.7%) | 2 (8.0%) |
| >90 minutes | 1 (10.0%) | 0 (0.0%) | 1 (4.0%) |
| missing | 2 (20.0%) | 1 (6.7%) | 3 (12.0%) |
| Once | 10 (7.6%) | 13 (7.8%) | 23 (7.7%) |
| 0-30 minutes | 10 (100%) | 11 (84.6%) | 21 (91.3%) |
| 31-60 minutes | 0 (0.0%) | 1 (7.7%) | 1 (4.3%) |
| >90 minutes | 0 (0.0%) | 1 (7.7%) | 1 (4.3%) |
| Twice | 19 (14.4%) | 24 (14.4%) | 43 (14.4%) |
| 0-30 minutes | 18 (94.7%) | 21 (87.5%) | 39 (90.7%) |
| 31-60 minutes | 1 (5.3%) | 3 (12.5%) | 4 (9.3%) |

TABLE 8
Summary of specific adverse events (Binary) by their
respective categorized maximum time to Onset
during Month 3-6 of Maintenance Phase by AE week 1
occurrence (Studies TRD3003 and TRD3004)

| | modal dose | modal dose | |
|---|---|---|---|
| | 56 | 84 | Total |
| Blood pressure increased | | | |
| (clinician-reported) | | | |
| AE week 1 occurrence | | | |
| N | 13 (13.1%) | 12 (11.5%) | 25 (12.3%) |
| None | 9 (9.1%) | 8 (7.7%) | 17 (8.4%) |
| 31-60 minutes | 6 (66.7%) | 7 (87.5%) | 13 (76.5%) |
| 61-90 minutes | 1 (11.1%) | 0 (0.0%) | 1 (5.9%) |
| missing | 2 (22.2%) | 1 (12.5%) | 3 (17.6%) |
| Once | 2 (2.0%) | 2 (1.9%) | 4 (2.0%) |
| 31-60 minutes | 2 (100%) | 2 (100%) | 4 (100%) |
| Twice | 2 (2.0%) | 2 (1.9%) | 4 (2.0%) |
| 31-60 minutes | 2 (100%) | 1 (50.0%) | 3 (75.0%) |
| 61-90 minutes | 0 (0.0%) | 1 (50.0%) | 1 (25.0%) |
| Dissociation(clinician-reported) | | | |
| AE week 1 occurrence | | | |
| N | 34 (34.3%) | 52 (50.0%) | 86 (42.4%) |
| None | 17 (17.2%) | 19 (18.3%) | 36 (17.7%) |
| 0-30 minutes | 11 (64.7%) | 13 (68.4%) | 24 (66.7%) |
| 31-60 minutes | 4 (23.5%) | 5 (26.3%) | 9 (25.0%) |
| 61-90 minutes | 1 (5.9%) | 0 (0.0%) | 1 (2.8%) |
| missing | 1 (5.9%) | 1 (5.3%) | 2 (5.6%) |
| Once | 5 (5.1%) | 12 (11.5%) | 17 (8.4%) |
| 0-30 minutes | 5 (100%) | 11 (91.7%) | 16 (94.1%) |
| 61-90 minutes | 0 (0.0%) | 1 (8.3%) | 1 (5.9%) |
| Twice | 12 (12.1%) | 21 (20.2%) | 33 (16.3%) |
| 0-30 minutes | 8 (66.7%) | 15 (71.4%) | 23 (69.7%) |
| 31-60 minutes | 4 (33.3%) | 5 (23.8%) | 9 (27.3%) |
| >90 minutes | 0 (0.0%) | 1 (4.8%) | 1 (3.0%) |
| Dizziness(clinician-reported) | | | |
| AE week 1 occurrence | | | |
| N | 55 (55.6%) | 38 (36.5%) | 93 (45.8%) |
| None | 23 (23.2%) | 11 (10.6%) | 34 (16.7%) |
| 0-30 minutes | 20 (87.0%) | 7 (63.6%) | 27 (79.4%) |
| 31-60 minutes | 1 (4.3%) | 2 (18.2%) | 3 (8.8%) |
| >90 minutes | 1 (4.3%) | 0 (0.0%) | 1 (2.9%) |
| missing | 1 (4.3%) | 2 (18.2%) | 3 (8.8%) |
| Once | 15 (15.2%) | 9 (8.7%) | 24 (11.8%) |
| 0-30 minutes | 14 (93.3%) | 7 (77.8%) | 21 (87.5%) |
| 31-60 minutes | 0 (0.0%) | 2 (22.2%) | 2 (8.3%) |
| missing | 1 (6.7%) | 0 (0.0%) | 1 (4.2%) |
| Twice | 17 (17.2%) | 18 (17.3%) | 35 (17.2%) |
| 0-30 minutes | 16 (94.1%) | 12 (66.7%) | 28 (80.0%) |
| 31-60 minutes | 1 (5.9%) | 5 (27.8%) | 6 (17.1%) |
| 61-90 minutes | 0 (0.0%) | 1 (5.6%) | 1 (2.9%) |
| Sedation(clinician-reported) | | | |
| AE week 1 occurrence | | | |
| N | 9 (9.1%) | 12 (11.5%) | 21 (10.3%) |
| None | 5 (5.1%) | 12 (11.5%) | 17 (8.4%) |
| 0-30 minutes | 3 (60.0%) | 8 (66.7%) | 11 (64.7%) |
| 31-60 minutes | 1 (20.0%) | 1 (8.3%) | 2 (11.8%) |
| 61-90 minutes | 0 (0.0%) | 1 (8.3%) | 1 (5.9%) |
| >90 minutes | 0 (0.0%) | 1 (8.3%) | 1 (5.9%) |
| missing | 1 (20.0%) | 1 (8.3%) | 2 (11.8%) |
| Twice | 4 (4.0%) | 0 | 4 (2.0%) |
| 0-30 minutes | 3 (75.0%) | 0 | 3 (75.0%) |
| 31-60 minutes | 1 (25.0%) | 0 | 1 (25.0%) |
| Vertigo (clinician-reported) | | | |
| AE week 1 occurrence | | | |
| N | 23 (23.2%) | 26 (25.0%) | 49 (24.1%) |
| None | 10 (10.1%) | 8 (7.7%) | 18 (8.9%) |
| 0-30 minutes | 7 (70.0%) | 5 (62.5%) | 12 (66.7%) |
| 31-60 minutes | 0 (0.0%) | 1 (12.5%) | 1 (5.6%) |
| 61-90 minutes | 1 (10.0%) | 1 (12.5%) | 2 (11.1%) |
| >90 minutes | 0 (0.0%) | 1 (12.5%) | 1 (5.6%) |
| missing | 2 (20.0%) | 0 (0.0%) | 2 (11.1%) |
| Once | 1 (1.0%) | 5 (4.8%) | 6 (3.0%) |
| 0-30 minutes | 1 (100%) | 4 (80.0%) | 5 (83.3%) |
| 61-90 minutes | 0 (0.0%) | 1 (20.0%) | 1 (16.7%) |
| Twice | 12 (12.1%) | 13 (12.5%) | 25 (12.3%) |
| 0-30 minutes | 11 (91.7%) | 11 (84.6%) | 22 (88.0%) |
| 31-60 minutes | 0 (0.0%) | 2 (15.4%) | 2 (8.0%) |
| 61-90 minutes | 1 (8.3%) | 0 (0.0%) | 1 (4.0%) |

TABLE 9
Summary of specific adverse events (Binary) by their
respective categorized maximum time to Onset
during Month 3-6 of Maintenance Phase by AE week 4
occurrence (Studies TRD3003 and TRD3004)

| | modal dose | modal dose | |
|---|---|---|---|
| | 56 | 84 | Total |
| Blood pressure increased | | | |
| (clinician-reported) | | | |
| AE week 4 occurrence | | | |
| N | 13 (13.1%) | 12 (11.5%) | 25 (12.3%) |
| None | 9 (9.1%) | 8 (7.7%) | 17 (8.4%) |
| 31-60 minutes | 6 (66.7%) | 7 (87.5%) | 13 (76.5%) |
| 61-90 minutes | 1 (11.1%) | 0 (0.0%) | 1 (5.9%) |
| missing | 2 (22.2%) | 1 (12.5%) | 3 (17.6%) |
| Once | 2 (2.0%) | 3 (2.9%) | 5 (2.5%) |
| 31-60 minutes | 2 (100%) | 3 (100%) | 5 (100%) |
| Twice | 2 (2.0%) | 1 (1.0%) | 3 (1.5%) |
| 31-60 minutes | 2 (100%) | 0 (0.0%) | 2 (66.7%) |
| 61-90 minutes | 0 (0.0%) | 1 (100%) | 1 (33.3%) |
| Dissociation(clinician-reported) | | | |
| AE week 4 occurrence | | | |
| N | 34 (34.3%) | 52 (50.0%) | 86 (42.4%) |
| None | 18 (18.2%) | 20 (19.2%) | 38 (18.7%) |
| 0-30 minutes | 11 (61.1%) | 14 (70.0%) | 25 (65.8%) |
| 31-60 minutes | 5 (27.8%) | 5 (25.0%) | 10 (26.3%) |
| 61-90 minutes | 1 (5.6%) | 0 (0.0%) | 1 (2.6%) |
| missing | 1 (5.6%) | 1 (5.0%) | 2 (5.3%) |
| Once | 5 (5.1%) | 10 (9.6%) | 15 (7.4%) |
| 0-30 minutes | 4 (80.0%) | 9 (90.0%) | 13 (86.7%) |
| 31-60 minutes | 1 (20.0%) | 0 (0.0%) | 1 (6.7%) |
| >90 minutes | 0 (0.0%) | 1 (10.0%) | 1 (6.7%) |
| Twice | 11 (11.1%) | 22 (21.2%) | 33 (16.3%) |
| 0-30 minutes | 9 (81.8%) | 16 (72.7%) | 25 (75.8%) |
| 31-60 minutes | 2 (18.2%) | 5 (22.7%) | 7 (21.2%) |
| 61-90 minutes | 0 (0.0%) | 1 (4.5%) | 1 (3.0%) |
| Dizziness(clinician-reported) | | | |
| AE week 4 occurrence | | | |
| N | 55 (55.6%) | 38 (36.5%) | 93 (45.8%) |
| None | 29 (29.3%) | 18 (17.3%) | 47 (23.2%) |
| 0-30 minutes | 25 (86.2%) | 11 (61.1%) | 36 (76.6%) |
| 31-60 minutes | 1 (3.4%) | 5 (27.8%) | 6 (12.8%) |
| >90 minutes | 1 (3.4%) | 0 (0.0%) | 1 (2.1%) |
| missing | 2 (6.9%) | 2 (11.1%) | 4 (8.5%) |
| Once | 7 (7.1%) | 6 (5.8%) | 13 (6.4%) |
| 0-30 minutes | 6 (85.7%) | 5 (83.3%) | 11 (84.6%) |
| 31-60 minutes | 1 (14.3%) | 1 (16.7%) | 2 (15.4%) |
| Twice | 19 (19.2%) | 14 (13.5%) | 33 (16.3%) |
| 0-30 minutes | 19 (100%) | 10 (71.4%) | 29 (87.9%) |
| 31-60 minutes | 0 (0.0%) | 3 (21.4%) | 3 (9.1%) |
| 61-90 minutes | 0 (0.0%) | 1 (7.1%) | 1 (3.0%) |
| Sedation(clinician-reported) | | | |
| AE week 4 occurrence | | | |
| N | 9 (9.1%) | 12 (11.5%) | 21 (10.3%) |
| None | 5 (5.1%) | 9 (8.7%) | 14 (6.9%) |
| 0-30 minutes | 3 (60.0%) | 6 (66.7%) | 9 (64.3%) |
| 31-60 minutes | 1 (20.0%) | 1 (11.1%) | 2 (14.3%) |
| 61-90 minutes | 0 (0.0%) | 1 (11.1%) | 1 (7.1%) |
| >90 minutes | 0 (0.0%) | 1 (11.1%) | 1 (7.1%) |
| missing | 1 (20.0%) | 0 (0.0%) | 1 (7.1%) |
| Once | 1 (1.0%) | 2 (1.9%) | 3 (1.5%) |
| 0-30 minutes | 0 (0.0%) | 2 (100%) | 2 (66.7%) |
| 31-60 minutes | 1 (100%) | 0 (0.0%) | 1 (33.3%) |
| Twice | 3 (3.0%) | 1 (1.0%) | 4 (2.0%) |
| 0-30 minutes | 3 (100%) | 0 (0.0%) | 3 (75.0%) |
| missing | 0 (0.0%) | 1 (100%) | 1 (25.0%) |
| Vertigo(clinician-reported) | | | |
| AE week 4 occurrence | | | |
| N | 23 (23.2%) | 26 (25.0%) | 49 (24.1%) |
| None | 12 (12.1%) | 10 (9.6%) | 22 (10.8%) |
| 0-30 minutes | 9 (75.0%) | 6 (60.0%) | 15 (68.2%) |
| 31-60 minutes | 0 (0.0%) | 2 (20.0%) | 2 (9.1%) |
| 61-90 minutes | 1 (8.3%) | 1 (10.0%) | 2 (9.1%) |
| >90 minutes | 0 (0.0%) | 1 (10.0%) | 1 (4.5%) |
| missing | 2 (16.7%) | 0 (0.0%) | 2 (9.1%) |
| Once | 3 (3.0%) | 3 (2.9%) | 6 (3.0%) |
| 0-30 minutes | 3 (100%) | 3 (100%) | 6 (100%) |
| Twice | 8 (8.1%) | 13 (12.5%) | 21 (10.3%) |
| 0-30 minutes | 7 (87.5%) | 11 (84.6%) | 18 (85.7%) |
| 31-60 minutes | 0 (0.0%) | 1 (7.7%) | 1 (4.8%) |
| 61-90 minutes | 1 (12.5%) | 1 (7.7%) | 2 (9.5%) |

TABLE 10
Summary of specific adverse events (Binary) by their
respective categorized maximum time to Onset during
Month 6-12 of Maintenance Phase by AE week 1
occurrence (Studies TRD3003 and TRD3004)

| | modal dose | modal dose | |
|---|---|---|---|
| | 56 | 84 | Total |
| Blood pressure increased | | | |
| (clinician-reported) | | | |
| AE week 1 occurrence | | | |
| N | 7 (10.4%) | 6 (10.0%) | 13 (10.2%) |
| None | 6 (9.0%) | 4 (6.7%) | 10 (7.9%) |
| 31-60 minutes | 5 (83.3%) | 3 (75.0%) | 8 (80.0%) |
| 61-90 minutes | 0 (0.0%) | 1 (25.0%) | 1 (10.0%) |
| missing | 1 (16.7%) | 0 (0.0%) | 1 (10.0%) |
| Once | 1 (1.5%) | 0 | 1 (0.8%) |
| 31-60 minutes | 1 (100%) | 0 | 1 (100%) |
| Twice | 0 | 2 (3.3%) | 2 (1.6%) |
| 31-60 minutes | 0 | 2 (100%) | 2 (100%) |
| Dissociation(clinician-reported) | | | |
| AE week 1 occurrence | | | |
| N | 19 (28.4%) | 33 (55.0%) | 52 (40.9%) |
| None | 9 (13.4%) | 13 (21.7%) | 22 (17.3%) |
| 0-30 minutes | 5 (55.6%) | 12 (92.3%) | 17 (77.3%) |
| 31-60 minutes | 3 (33.3%) | 1 (7.7%) | 4 (18.2%) |
| 61-90 minutes | 1 (11.1%) | 0 (0.0%) | 1 (4.5%) |
| Once | 4 (6.0%) | 8 (13.3%) | 12 (9.4%) |
| 0-30 minutes | 3 (75.0%) | 6 (75.0%) | 9 (75.0%) |
| 31-60 minutes | 0 (0.0%) | 2 (25.0%) | 2 (16.7%) |
| missing | 1 (25.0%) | 0 (0.0%) | 1 (8.3%) |
| Twice | 6 (9.0%) | 12 (20.0%) | 18 (14.2%) |
| 0-30 minutes | 3 (50.0%) | 8 (66.7%) | 11 (61.1%) |
| 31-60 minutes | 3 (50.0%) | 4 (33.3%) | 7 (38.9%) |
| Dizziness(clinician-reported) | | | |
| AE week 1 occurrence | | | |
| N | 28 (41.8%) | 20 (33.3%) | 48 (37.8%) |
| None | 11 (16.4%) | 7 (11.7%) | 18 (14.2%) |
| 0-30 minutes | 6 (54.5%) | 3 (42.9%) | 9 (50.0%) |
| 31-60 minutes | 4 (36.4%) | 2 (28.6%) | 6 (33.3%) |
| missing | 1 (9.1%) | 2 (28.6%) | 3 (16.7%) |
| Once | 10 (14.9%) | 6 (10.0%) | 16 (12.6%) |
| 0-30 minutes | 10 (100%) | 5 (83.3%) | 15 (93.8%) |
| 31-60 minutes | 0 (0.0%) | 1 (16.7%) | 1 (6.3%) |
| Twice | 7 (10.4%) | 7 (11.7%) | 14 (11.0%) |
| 0-30 minutes | 7 (100%) | 6 (85.7%) | 13 (92.9%) |
| >90 minutes | 0 (0.0%) | 1 (14.3%) | 1 (7.1%) |
| Sedation(clinician-reported) | | | |
| AE week 1 occurrence | | | |
| N | 9 (13.4%) | 6 (10.0%) | 15 (11.8%) |
| None | 5 (7.5%) | 5 (8.3%) | 10 (7.9%) |
| 0-30 minutes | 3 (60.0%) | 4 (80.0%) | 7 (70.0%) |
| 31-60 minutes | 1 (20.0%) | 0 (0.0%) | 1 (10.0%) |
| missing | 1 (20.0%) | 1 (20.0%) | 2 (20.0%) |
| Twice | 4 (6.0%) | 1 (1.7%) | 5 (3.9%) |
| 0-30 minutes | 3 (75.0%) | 1 (100%) | 4 (80.0%) |
| 31-60 minutes | 1 (25.0%) | 0 (0.0%) | 1 (20.0%) |
| Vertigo(clinician-reported) | | | |
| AE week 1 occurrence | | | |
| N | 18 (26.9%) | 12 (20.0%) | 30 (23.6%) |
| None | 8 (11.9%) | 1 (1.7%) | 9 (7.1%) |
| 0-30 minutes | 5 (62.5%) | 1 (100%) | 6 (66.7%) |
| 31-60 minutes | 1 (12.5%) | 0 (0.0%) | 1 (11.1%) |
| 61-90 minutes | 1 (12.5%) | 0 (0.0%) | 1 (11.1%) |
| missing | 1 (12.5%) | 0 (0.0%) | 1 (11.1%) |
| Once | 0 | 5 (8.3%) | 5 (3.9%) |
| 0-30 minutes | 0 | 2 (40.0%) | 2 (40.0%) |
| 31-60 minutes | 0 | 2 (40.0%) | 2 (40.0%) |
| missing | 0 | 1 (20.0%) | 1 (20.0%) |
| Twice | 10 (14.9%) | 6 (10.0%) | 16 (12.6%) |
| 0-30 minutes | 9 (90.0%) | 5 (83.3%) | 14 (87.5%) |
| 31-60 minutes | 1 (10.0%) | 1 (16.7%) | 2 (12.5%) |

TABLE 11
Summary of specific adverse events (Binary) by their
respective categorized maximum time to Onset during
Month 6-12 of Maintenance Phase by AE week 4
occurrence (Studies TRD3003 and TRD3004)

| | modal dose | modal dose | |
|---|---|---|---|
| | 56 | 84 | Total |
| Blood pressure increased | | | |
| (clinician-reported) | | | |
| AE week 4 occurrence | | | |
| N | 7 (10.4%) | 6 (10.0%) | 13 (10.2%) |
| None | 5 (7.5%) | 3 (5.0%) | 8 (6.3%) |
| 31-60 minutes | 4 (80.0%) | 2 (66.7%) | 6 (75.0%) |
| 61-90 minutes | 0 (0.0%) | 1 (33.3%) | 1 (12.5%) |
| missing | 1 (20.0%) | 0 (0.0%) | 1 (12.5%) |
| Once | 1 (1.5%) | 2 (3.3%) | 3 (2.4%) |
| 31-60 minutes | 1 (100%) | 2 (100%) | 3 (100%) |
| Twice | 1 (1.5%) | 1 (1.7%) | 2 (1.6%) |
| 31-60 minutes | 1 (100%) | 1 (100%) | 2 (100%) |
| Dissociation(clinician-reported) | | | |
| AE week 4 occurrence | | | |
| N | 19 (28.4%) | 33 (55.0%) | 52 (40.9%) |
| None | 11 (16.4%) | 13 (21.7%) | 24 (18.9%) |
| 0-30 minutes | 6 (54.5%) | 11 (84.6%) | 17 (70.8%) |
| 31-60 minutes | 3 (27.3%) | 2 (15.4%) | 5 (20.8%) |
| 61-90 minutes | 1 (9.1%) | 0 (0.0%) | 1 (4.2%) |
| missing | 1 (9.1%) | 0 (0.0%) | 1 (4.2%) |
| Once | 1 (1.5%) | 8 (13.3%) | 9 (7.1%) |
| 0-30 minutes | 0 (0.0%) | 7 (87.5%) | 7 (77.8%) |
| 31-60 minutes | 1 (100%) | 1 (12.5%) | 2 (22.2%) |
| Twice | 7 (10.4%) | 12 (20.0%) | 19 (15.0%) |
| 0-30 minutes | 5 (71.4%) | 8 (66.7%) | 13 (68.4%) |
| 31-60 minutes | 2 (28.6%) | 4 (33.3%) | 6 (31.6%) |
| Dizziness(clinician-reported) | | | |
| AE week 4 occurrence | | | |
| N | 28 (41.8%) | 20 (33.3%) | 48 (37.8%) |
| None | 15 (22.4%) | 7 (11.7%) | 22 (17.3%) |
| 0-30 minutes | 10 (66.7%) | 3 (42.9%) | 13 (59.1%) |
| 31-60 minutes | 4 (26.7%) | 2 (28.6%) | 6 (27.3%) |
| missing | 1 (6.7%) | 2 (28.6%) | 3 (13.6%) |
| Once | 4 (6.0%) | 5 (8.3%) | 9 (7.1%) |
| 0-30 minutes | 4 (100%) | 5 (100%) | 9 (100%) |
| Twice | 9 (13.4%) | 8 (13.3%) | 17 (13.4%) |
| 0-30 minutes | 9 (100%) | 6 (75.0%) | 15 (88.2%) |
| 31-60 minutes | 0 (0.0%) | 1 (12.5%) | 1 (5.9%) |
| >90 minutes | 0 (0.0%) | 1 (12.5%) | 1 (5.9%) |
| Sedation(clinician-reported) | | | |
| AE week 4 occurrence | | | |
| N | 9 (13.4%) | 6 (10.0%) | 15 (11.8%) |
| None | 4 (6.0%) | 4 (6.7%) | 8 (6.3%) |
| 0-30 minutes | 2 (50.0%) | 3 (75.0%) | 5 (62.5%) |
| 31-60 minutes | 1 (25.0%) | 0 (0.0%) | 1 (12.5%) |
| missing | 1 (25.0%) | 1 (25.0%) | 2 (25.0%) |
| Once | 2 (3.0%) | 2 (3.3%) | 4 (3.1%) |
| 0-30 minutes | 1 (50.0%) | 2 (100%) | 3 (75.0%) |
| 31-60 minutes | 1 (50.0%) | 0 (0.0%) | 1 (25.0%) |
| Twice | 3 (4.5%) | 0 | 3 (2.4%) |
| 0-30 minutes | 3 (100%) | 0 | 3 (100%) |
| Vertigo(clinician-reported) | | | |
| AE week 4 occurrence | | | |
| N | 18 (26.9%) | 12 (20.0%) | 30 (23.6%) |
| None | 7 (10.4%) | 4 (6.7%) | 11 (8.7%) |
| 0-30 minutes | 5 (71.4%) | 3 (75.0%) | 8 (72.7%) |
| 31-60 minutes | 1 (14.3%) | 1 (25.0%) | 2 (18.2%) |
| missing | 1 (14.3%) | 0 (0.0%) | 1 (9.1%) |
| Once | 2 (3.0%) | 2 (3.3%) | 4 (3.1%) |
| 0-30 minutes | 1 (50.0%) | 1 (50.0%) | 2 (50.0%) |
| 31-60 minutes | 0 (0.0%) | 1 (50.0%) | 1 (25.0%) |
| 61-90 minutes | 1 (50.0%) | 0 (0.0%) | 1 (25.0%) |
| Twice | 9 (13.4%) | 6 (10.0%) | 15 (11.8%) |
| 0-30 minutes | 8 (88.9%) | 4 (66.7%) | 12 (80.0%) |
| 31-60 minutes | 1 (11.1%) | 1 (16.7%) | 2 (13.3%) |
| missing | 0 (0.0%) | 1 (16.7%) | 1 (6.7%) |

Tables 12-16 include the incidences of clinician-reported blood pressure (Table 12), clinician-reported dissociation (Table 13), dizziness (Table 14), sedation (Table 15), and vertigo (Table 16) spanning week 1 to month 12 using the data of Example 2. These data show that adverse events, particularly dissociation and increase in blood pressure, generally peak at 40 minutes.

TABLE 12
Blood Pressure (clinician)

| | | TOTAL | 0-30 m | 31-60 m | 61-90 m | >90 m | missing | n >60 m | % >60 m |
|---|---|---|---|---|---|---|---|---|---|
| Based on Week 1 | | | | | | | | | |
| Week 1 incidence | None | 21 | | 20 | 1 | | | 1 | 4.8 |
| Weeks 2-4 | Once | 13 | 1 | 11 | 1 | | | 1 | 7.7 |
| | Twice | 14 | | 12 | 1 | 1 | | 2 | 14.2 |
| Week 1 incidence | None | 17 | 1 | 12 | 2 | | 2 | 2 | 11.8 |
| Month 2 | Once | 9 | | 9 | | | | 0 | 0 |
| | Twice | 10 | | 10 | | | | 0 | 0 |
| Week 1 incidence | None | 17 | | 13 | 1 | | 3 | 1 | 5.9 |
| Month 3-6 | Once | 4 | | 4 | | | | 0 | 0 |
| | Twice | 4 | | 3 | 1 | | | 1 | 25 |
| Week 1 incidence | None | 10 | | 8 | 1 | | 1 | 1 | 10 |
| Month 6-12 | Once | 1 | | 1 | | | | 0 | 0 |
| | Twice | 2 | | 2 | | | | 0 | 0 |
| Based on Week 4 | | | | | | | | | |
| Week 1 incidence | None | 18 | 1 | 13 | 2 | | 2 | 2 | 11.1 |
| Month 2 | Once | 9 | | 9 | | | | 0 | 0 |
| | Twice | 9 | | 9 | | | | 0 | 0 |
| Week 1 incidence | None | 17 | | 13 | 1 | | 3 | 1 | 5.9 |
| Month 3-6 | Once | 5 | | 5 | | | | 0 | 0 |
| | Twice | 3 | | 2 | 1 | | | 1 | 33.3 |
| Week 1 incidence | None | 8 | | 6 | 1 | | 1 | 1 | 12.5 |
| Month 6-12 | Once | 3 | | 3 | | | | 0 | 0 |
| | Twice | 2 | | 2 | | | | 0 | 0 |

TABLE 13
Dissociation (clinician)

| | | TOTAL | 0-30 m | 31-60 m | 61-90 m | >90 m | missing | n >60 m | % >60 m |
|---|---|---|---|---|---|---|---|---|---|
| Based on Week 1 | | | | | | | | | |
| Week 1 incidence | None | 44 | 34 | 8 | | | 2 | 0 | 0 |
| Weeks 2-4 | Once | 41 | 29 | 9 | 1 | 2 | | 3 | 7.3 |
| | Twice | 64 | 44 | 16 | 4 | | | 4 | 6.3 |
| Week 1 incidence | None | 46 | 33 | 8 | | | 5 | 0 | 0 |
| Month 2 | Once | 25 | 21 | 3 | 1 | | | 1 | 4 |
| | Twice | 45 | 35 | 10 | | | | 0 | 0 |
| Week 1 incidence | None | 36 | 24 | 9 | 1 | | 2 | 1 | 2.8 |
| Month 3-6 | Once | 17 | 16 | 1 | | | | 0 | 0 |
| | Twice | 33 | 23 | 9 | | 1 | | 1 | 3 |
| Week 1 incidence | None | 22 | 17 | 4 | 1 | | | 1 | 4.5 |
| Month 6-12 | Once | 12 | 9 | 2 | | | 1 | 0 | 0 |
| | Twice | 18 | 11 | 7 | | | | 0 | 0 |
| Based on Week 4 | | | | | | | | | |
| Week 1 incidence | None | 46 | 33 | 8 | | | 5 | 0 | 0 |
| Month 2 | Once | 21 | 18 | 2 | 1 | | | 1 | 4.8 |
| | Twice | 49 | 38 | 11 | | | | 0 | 0 |
| Week 1 incidence | None | 38 | 25 | 10 | 1 | | 2 | 1 | 2.6 |
| Month 3-6 | Once | 15 | 13 | 1 | | 1 | | 1 | 6.7 |
| | Twice | 33 | 25 | 7 | 1 | | | 1 | 3 |
| Week 1 incidence | None | 24 | 17 | 5 | 1 | | 1 | 1 | 4.2 |
| Month 6-12 | Once | 9 | 7 | 2 | | | | 0 | 0 |
| | Twice | 19 | 13 | 6 | | | | 0 | 0 |

TABLE 14
Dizziness

| | | TOTAL | 0-30 m | 31-60 m | 61-90 m | >90 m | missing | n >60 m | % >60 m |
|---|---|---|---|---|---|---|---|---|---|
| Based on Week 1 | | | | | | | | | |
| Week 1 incidence | None | 46 | 35 | 5 | 2 | | 4 | 2 | 4.3 |
| Weeks 2-4 | Once | 56 | 41 | 8 | 2 | 1 | 4 | 3 | 5.4 |
| | Twice | 76 | 60 | 9 | 4 | 2 | 1 | 6 | 7.9 |
| Week 1 incidence | None | 27 | 21 | 2 | 1 | | 3 | 1 | 3.7 |
| Month 2 | Once | 33 | 27 | 2 | | 2 | 2 | 2 | 6.1 |
| | Twice | 55 | 49 | 2 | 3 | | 1 | 3 | 5.5 |
| Week 1 incidence | None | 34 | 27 | 3 | | 1 | 3 | 1 | 2.9 |
| Month 3-6 | Once | 24 | 21 | 2 | | | 1 | 0 | 0 |
| | Twice | 35 | 28 | 6 | 1 | | | 1 | 2.9 |
| Week 1 incidence | None | 18 | 9 | 6 | | | 3 | 0 | 0 |
| Month 6-12 | Once | 16 | 15 | 1 | | | | 0 | 0 |
| | Twice | 14 | 13 | | | 1 | | 1 | 7.1 |
| Based on Week 4 | | | | | | | | | |
| Week 1 incidence | None | 35 | 26 | 2 | 2 | 1 | 4 | 3 | 8.6 |
| Month 2 | Once | 27 | 24 | 2 | 1 | | | 1 | 3.7 |
| | Twice | 53 | 47 | 2 | 1 | 1 | 2 | 2 | 3.8 |
| Week 1 incidence | None | 47 | 36 | 6 | | 1 | 4 | 1 | 2.1 |
| Month 3-6 | Once | 13 | 11 | 2 | | | | 0 | 0 |
| | Twice | 33 | 29 | 3 | 1 | | | 1 | 3 |
| Week 1 incidence | None | 22 | 13 | 6 | | | 3 | 0 | 0 |
| Month 6-12 | Once | 9 | 9 | | | | | 0 | 0 |
| | Twice | 17 | 15 | 1 | | 1 | | 1 | 5.9 |

TABLE 15
Sedation

| | | TOTAL | 0-30 m | 31-60 m | 61-90 m | >90 m | missing | n >60 m | % >60 m |
|---|---|---|---|---|---|---|---|---|---|
| Based on Week 1 | | | | | | | | | |
| Week 1 incidence | None | 19 | 9 | 4 | 1 | 2 | 3 | 3 | 15.8 |
| Weeks 2-4 | Once | 7 | 1 | 1 | 3 | 1 | 1 | 4 | 57.1 |
| | Twice | 12 | 7 | 2 | 2 | | 1 | 2 | 16.7 |
| Week 1 incidence | None | 11 | 9 | | | | 2 | 0 | 0 |
| Month 2 | Once | 4 | 3 | 1 | | | | 0 | 0 |
| | Twice | 9 | 7 | 2 | | | | 0 | 0 |
| Week 1 incidence | None | 17 | 11 | 2 | 1 | 1 | 2 | 2 | 11.8 |
| Month 3-6 | Once | 0 | | | | | | 0 | 0 |
| | Twice | 4 | 3 | 1 | | | | 0 | 0 |
| Week 1 incidence | None | 10 | 7 | 1 | | | 2 | 0 | 0 |
| Month 6-12 | Once | 0 | | | | | | 0 | 0 |
| | Twice | 5 | 4 | 1 | | | | 0 | 0 |
| Based on Week 4 | | | | | | | | | |
| Week 1 incidence | None | 10 | 9 | | | | 1 | 0 | 0 |
| Month 2 | Once | 5 | 5 | | | | | 0 | 0 |
| | Twice | 9 | 5 | 3 | | | 1 | 0 | 0 |
| Week 1 incidence | None | 14 | 9 | 2 | 1 | 1 | 1 | 2 | 14.3 |
| Month 3-6 | Once | 3 | 2 | 1 | | | | 0 | 0 |
| | Twice | 4 | 3 | | | | 1 | 0 | 0 |
| Week 1 incidence | None | 8 | 5 | 1 | | | 2 | 0 | 0 |
| Month 6-12 | Once | 4 | 3 | 1 | | | | 0 | 0 |
| | Twice | 3 | 3 | | | | | 0 | 0 |

TABLE 16
Vertigo

| | | TOTAL | 0-30 m | 31-60 m | 61-90 m | >90 m | missing | n >60 m | % >60 m |
|---|---|---|---|---|---|---|---|---|---|
| Based on Week 1 | | | | | | | | | |
| Week 1 incidence | None | 45 | 31 | 7 | 2 | 3 | 2 | 5 | 11.1 |
| Weeks 2-4 | Once | 29 | 19 | 7 | 1 | 2 | | 3 | 10.3 |
| | Twice | 65 | 57 | 5 | 1 | 2 | | 3 | 4.6 |
| Week 1 incidence | None | 24 | 18 | 2 | 1 | | 3 | 1 | 4.2 |
| Month 2 | Once | 19 | 14 | 2 | 1 | 2 | | 3 | 15.8 |
| | Twice | 48 | 46 | 2 | | | | 0 | 0 |
| Week 1 incidence | None | 18 | 12 | 1 | 2 | 1 | 2 | 3 | 16.7 |
| Month 3-6 | Once | 6 | 5 | | 1 | | | 1 | 16.7 |
| | Twice | 25 | 22 | 2 | 1 | | | 1 | 4 |
| Week 1 incidence | None | 9 | 6 | 1 | 1 | | 1 | 1 | 11.1 |
| Month 6-12 | Once | 5 | 2 | 2 | | | 1 | 0 | 0 |
| | Twice | 16 | 14 | 2 | | | | 0 | 0 |
| Based on Week 4 | | | | | | | | | |
| Week 1 incidence | None | 25 | 18 | 1 | 2 | 1 | 3 | 3 | 12 |
| Month 2 | Once | 23 | 21 | 1 | | 1 | | 1 | 4.3 |
| | Twice | 43 | 39 | 4 | | | | 0 | 0 |
| Week 1 incidence | None | 22 | 15 | 2 | 2 | 1 | 2 | 3 | 13.6 |
| Month 3-6 | Once | 6 | 6 | | | | | 0 | 0 |
| | Twice | 21 | 18 | 1 | 2 | | | 2 | 9.5 |
| Week 1 incidence | None | 11 | 8 | 2 | | | 1 | 0 | 0 |
| Month 6-12 | Once | 4 | 2 | 1 | 1 | | | 1 | 25 |
| | Twice | 15 | 12 | 2 | | | 1 | 0 | 0 |

### E. Discussion

This study revealed that the more frequently a given AE occurs during postdose monitoring early in ESK treatment, the more likely it is to recur in subsequent postdose monitoring sessions. Those patients who experience one of the most commonly-reported AEs once or twice during the first week of treatment are more likely to suffer a recurrence of the same AE compared with patients who do not. The five most common AEs associated with ESK plus oral antidepressant treatment of TRD (dissociation, dizziness, nausea, sedation, vertigo) are more likely to recur after Week 1 if they occurred more frequently during the first week of treatment. Reported severity of dissociation, dizziness, nausea, sedation, vertigo, and increased blood pressure were mostly mild and rarely severe, both in terms of initial incidence and recurrence. The incidences of clinician-reported dissociation, dizziness, nausea, and sedation were highest in Week 1 of treatment with ESK plus an oral antidepressant and decreased thereafter.

For each AE other than CADSS-defined dissociation, the majority of patients did not report the AEs in Week 1 and thus were stratified into the groups with the lowest recurrence risk (for CADSS defined dissociation, 43% of patients were in this group).

When AEs occurred during Week 1 and Week 4, recurrence of AEs after Week 4 (the end of the induction period) were more closely related to the frequency of the same AEs during Week 4 than during Week 1. Weeks 1 and 4 did not provide different insights if AEs occurred in neither. The predictive utility for both week 1 and week 4 AE frequency was generally stronger in the first 6 months. Incidence of specific AEs during weeks 1 or 4 was a far more useful prognostic indicator of future AE recurrence than ESK dose. The frequency of AEs that occurred during the postdose monitoring period of week 1 best predicted recurrence of AEs at subsequent treatment sessions for the remainder of the induction period (weeks 2-4) and generally predicted the lower recurrence of AEs thereafter.

Among participants for whom an AE was not spontaneously reported during week 1, incidence rates of that AE were lower than the overall rates in every subsequent time frame examined. Further, if given AEs are absent during week 1, the modest dose effects noted for some AEs are largely absent owing to the infrequent recurrence of those AEs. If a given AE was not reported in week 1 during the postdose monitoring periods, <10% of participants experienced that AE after any subsequent treatment session (exception was dizziness at 3-6 months [11.6%]).

Rates of recurrence generally diminish over time.

Differences between formally measured versus clinician-reported rates of dissociation, sedation, and abnormally elevated BP are evident. These measurement approaches are inherently calibrated to different standards: clinicians were encouraged to report any AE that they believed to be clinically significant or merited treatment, whereas formal measures simply detect deviations from a commonly accepted standard of ‘normal’ values. Thus, higher rates are, not surprisingly, revealed by the formal measures than by clinician report. However, even with these differences, the same fundamental relationships between early and later tolerability were observed across measurement modalities.

Although this study found that recurrence rates were lower in patients who had not previously experienced the same AE, recurrence rates of objectively-measured dissociation, sedation, and elevated BP never reached zero. Thus, BP must be assessed before ESK administration, at least at 40 minutes post-dose and thereafter as needed per clinical judgment, and patients must be monitored for two hours to ensure that sedation and dissociation are resolved prior to discharge.

### Example 3: Reduced Monitoring Procedure

This example summarizes a procedure for reduced monitoring as described herein.

Clinicians administering esketamine and implementing reduced monitoring for qualified patients should follow all requirements including, but not limited to:
Maintain records demonstrating processes/procedures are in place and being followed. This includes those regarding reduced monitoring for qualified patients.
Submit appropriate forms to the clinician for every qualified patient who is monitored less than the 2-hour minimum monitoring requirement, within 7 calendar days following administration of every dose

### A. Considerations for Identifying a Qualified Patient

The following criteria are used to identify patients who may be appropriate for reduced monitoring:
The patient is enrolled in the option to become a qualified patient and previously received at least 8 esketamine treatment sessions (i.e. beyond induction treatment) at the time reduced monitoring begins
The patient is receiving no more than 1 treatment session per week at the time reduced monitoring begins
The patient tolerated all esketamine treatment sessions and the associated side effects without the need for medical intervention, including emergency treatment, in any prior treatment sessions including:
No clinically meaningful blood pressure and heart rate increases observed
No more than mild dissociation observed
No more than mild sedation or disorientation observed
No more than mild nausea without vomiting observed
No other clinically relevant side effects of concern per clinical judgment
The patient does not have current uncontrolled hypertension in the context of either cardiovascular or endocrine co-morbidities
The patient has an adult caregiver/responsible adult present who agree to remain with the patient after the treatment session and to participate with the patient in a telephone assessment with the clinician to assess the patient's clinical status at an agreed upon time, at least 2 hours later the same day.
The adult caregiver/responsible adult agrees to provide their information (name, relationship, contact information) so that the follow-up telephone assessment can be arranged

### B. Informed Consent of the Patient and Adult Caregiver/Responsible Adult

Informed consent includes:
The patient and/or clinician review potential risks related to monitoring for less than 2 hours with the patient and caregiver/responsible adult, including but not limited to the following:
Ensuring patient and caregiver/responsible adult are aware of the potential risks and agree to proceed with administration and monitoring for less than 2 hours if appropriate. Informed consent should be documented at the healthcare setting.
The patient and adult caregiver/responsible adult agree to comply with the instructions provided by the clinician if the patient does not feel well or has any other relevant health issues following the observed monitoring period.
The adult caregiver/responsible adult agrees to remain with the patient after the treatment session has ended and until the agreed upon follow-up telephone assessment with the clinician has occurred, at least 2 hours after the end of the treatment session, later the same day

### C. Instructions for the Day of Administration

The reduced monitoring treatment option is only applicable for qualified patients being treated at the esketamine healthcare setting and is not to occur with in-home administration and monitoring of esketamine.

### Guidance on the Day of Administration

### (i) While the Patient is at the Healthcare Setting

Relevant forms are completed by the clinician monitoring the patient at the healthcare setting
The clinician continues to monitor the patient until clinically stable based on clinical judgment

To determine whether the patient is clinically stable, the clinician confirms the following clinical criteria are met during the treatment session:
No clinically meaningful blood pressure and heart rate increases observed
No more than mild dissociative symptoms observed which have resolved
No more than mild sedation observed which has resolved
No other clinically relevant side effects of concern per clinical judgment
If, based on the clinical judgment of the clinician, the patient is not ready to be discharged early, the healthcare setting accommodates the patient for a full two hours or greater if needed.
If the patient is determined to be clinically stable after being monitored for at least 1 hour but less than 2 hours, this should be documented.
Prior to discharge, the clinician confirms there is an adult caregiver/responsible adult present who agrees to remain with the patient and participate with the patient in a telephone assessment conducted with the HCP at least 2 hours after the end of the treatment session, later that same day.
The clinician provides instructions to the patient and adult caregiver/responsible adult based on the healthcare setting's processes and procedures, in the event the patient requires medical intervention after the patient and adult caregiver/responsible adult leave the healthcare setting

### After the HCP-Monitored Treatment Session Ends:

For those patients who are determined to be clinically stable and discharged earlier than 2 hours after esketamine administration, the clinician contacts the patient and adult caregiver/responsible adult at the agreed upon time, at least 2 hours after the treatment session has ended, to follow up on the patient's clinical status
During the caregiver/patient telephone assessment, the clinician specifically asks if the patient has any new sedation or dissociation that started since the treatment session ended. The clinician documents this on the form.
If the patient or adult caregiver/responsible adult reports any clinically relevant adverse events, the clinician should determine if treatment of the adverse event is warranted or if it is appropriate to schedule a follow-up telephone assessment the following day.
If the patient requires medical intervention, the patient and adult caregiver/responsible adult follow the instructions provided by the clinician which may include seeking emergency medical services and/or calling 911.
Any serious adverse events should be reported on any forms.
All other non-serious adverse events (other than dissociation or sedation) or product quality complaints associated with esketamine should be reported.
The clinician submits all forms for every patient within 7 calendar days following administration of every dose.
Patients agree not to engage in potentially hazardous activities, such as driving a motor vehicle or operating machinery until the next day, following a restful sleep.
The clinician agrees to stop reduced monitoring in future treatment sessions if the patient fails to comply with all required elements.

### D. Rationale for Proposed Elements

### (i) Determining Who May be an Appropriate Patient

By requiring patients to have had at least 8 previous treatment sessions, it is considered likely that their tolerability profile at subsequent dosing session can be anticipated.

In addition to requiring that the patient have no history of uncontrolled hypertension, the following requirements add further confidence that the patient has not had any clinically significant issues at prior treatment sessions: no clinically meaningful blood pressure and heart rate increases observed, no more than mild dissociation observed, no more than mild sedation or disorientation observed, no more than mild, self-limiting nausea without vomiting observed, and no other clinically relevant side effects of concern per clinical judgment.

### (ii) Duration of Post Dose Monitoring

In the current proposal the duration of the post-dose monitoring period will be based on the clinical stability per clinical judgment. However, it is required that all patients be monitored for at least 1-hour post dose.

As such, for an established patient who has previously received at least 8 treatment sessions with no significant clinical issues observed, a 1-hour minimum duration ensures that if a patient were to experience dissociation, sedation or blood pressure increase, those events would have started within the first hour post-dose. Thereafter, based on the current proposal, the monitoring period would continue until the patient was clinically stable.

If the patient is not ready to be discharged early, the patient will remain for the full 2 hours or longer as clinically needed.

1. A method of treating depression in a human patient in need thereof, said method having an induction phase and treatment sessions, wherein the induction phase has a duration of 4 weeks, the method comprising:
intranasally administering about 56 mg or about 84 mg of esketamine per induction phase treatment session to said patient, wherein the induction phase treatment session occurs at a frequency of twice weekly during the induction phase; wherein prior to any treatment session, the patient has a systolic blood pressure of less than 140 mmHg and a diastolic blood pressure of less than 110 mmHg; and
after at least the first two treatment sessions, monitoring the patient following each treatment session for a period of at least 90 minutes for adverse events; and, if the patient does not experience a severe adverse event and does not experience any of the following adverse events:
clinically meaningful blood pressure increases,
clinically meaningful heart rate increases,
moderate or greater levels of dissociation,
moderate or greater levels of sedation or disorientation, and
moderate or greater levels of nausea without vomiting,
following any two consecutive treatment sessions:
the patient becomes a qualified patient for a post-treatment session monitoring period of less than 90 minutes in a next treatment session.

2. The method of claim 1, wherein the patient becomes a qualified patient after at least the first three, four, five, six, seven, or eight treatment sessions.

3. The method of claim 1, wherein the patient becomes a qualified patient after eight treatment sessions.

4. The method of claim 1, wherein the qualified patient post-treatment session monitoring period is at least 60 minutes.

5. The method of claim 1, wherein a treatment session is rescheduled if, prior to the treatment session, the patient does not have a systolic blood pressure of less than 140 mmHg and a diastolic blood pressure of less than 110 mmHg.

6. The method of claim 1, wherein the patient does not have current uncontrolled hypertension.

7. The method of claim 1, wherein the patient is not taking medication or substances that promote sedation or blood pressure increases.

8. The method of claim 1, wherein the patient is less than 65 years of age.

9. The method of claim 1, wherein the depression is major depressive disorder.

10. The method of claim 9, wherein the depression is major depressive disorder with suicidal ideation or severe major depressive disorder.

11. The method of claim 1, wherein the method further comprises a subsequent maintenance phase comprising intranasally administering about 56 mg or about 84 mg of esketamine per maintenance phase treatment session to said patient, wherein the maintenance phase treatment session occurs at a frequency of once weekly for the first four weeks of the maintenance phase and is adjusted to once weekly or once every other week thereafter.

12. The method of claim 11, wherein the depression is major depressive disorder.

13. The method of claim 12, wherein the depression is treatment resistant depression.

14. The method of claim 1, comprising adjunctive treatment with a therapeutically effective amount of one or more antidepressants during the induction phase and the maintenance phase.

15. The method of claim 14, wherein the one or more antidepressants are administered at least 3 hours after any induction phase or maintenance phase treatment session.

16. The method of claim 1, wherein in the next treatment session the qualified patient is intranasally administering about 56 mg or about 84 mg of esketamine and monitored in the post-treatment session monitoring period for less than 90 minutes and released after a healthcare professional determines that the qualified patient is clinically stable and discharge ready.

17-32. (canceled)
